(19) United States
(12) Patent Application Publication
(10) Pub. No.: US 2022/0390457 A1
(43) Pub. Date: Dec. 8, 2022

(54) MEANS AND METHODS FOR MULTIPARAMETER CYTOMETRY-BASED LEUKOCYTE SUBSETTING

(71) Applicants: Stichting EuroFlow, Leiden (NL); Academisch Ziekenhuis Leiden h.o.d.n. LUMC, Leiden (NL); University of Salamanca, Salamanca (ES)

(72) Inventors: Jacobus Johannes Maria Van Dongen, Leiden (NL); José Alberto Orfao De Matos Correia E Vale, Salamanca (ES); Cristina Isabel Gonçalves Grunho Teodósio, Leiden (NL); Martín Pérez Y Andrés, Salamanca (ES); Julia Maria Almeida Parra, Salamanca (ES); Wouter Bernard Louis Van Den Bossche, Leiden (NL); Vitor Dessanti Botafogo Goncalves, Salamanca (ES); Magdalena Agnieszka Berkowska, Leiden (NL); Kyra Van Der Pan, Leiden (NL); Elena Blanco Álvarez, Salamanca (ES); Annieck Margo Diks, Leiden (NL); Daniela Pinto Damasceno, Salamanca (ES); Alejandro Hernández Delgado, Salamanca (ES)

(21) Appl. No.: 17/774,633

(22) PCT Filed: Nov. 5, 2020
(86) PCT No.: PCT/NL2020/050688

§ 371 Date: May 5, 2022

(30) Foreign Application Priority Data
Nov. 5, 2019 (NL) 2024163

(51) Int. Cl. G01N 33/574; G01N 33/569; G01N 33/58; G01N 33/68

(52) U.S. Cl. CPC G01N 33/57492; G01N 33/56972; G01N 33/582; G01N 33/6872; G01N 2800/52

(57) ABSTRACT

The invention relates to the field of diagnostic immunology. Provided are means and methods for multiparameter cytometry-based leukocyte subsetting, which is advantageously used for the monitoring of the immune status of a subject, and/or for monitoring the effects of an immune modulatory treatment. Provided among others is a reagent composition comprising antibodies conjugated to a detectable label, the conjugated antibodies being directed against the following combination of markers: CD141, HLA-DR, CD16, CD33, CD300e, CD303 and CD14, wherein the antibodies directed against CD300e and CD303 may be conjugated to the same label.

The invention relates to the field of diagnostics, in particular diagnostic immunology. Provided are means and methods for multiparameter cytometry-based leukocyte subsetting, which is advantageously used for the monitoring of the immune status of a subject, and/or for monitoring the effects of an immune modulatory treatment.

Detailed monitoring of the immune status and the effects of innovative immune modulatory treatments on immune cells is crucial. This is due to several reasons, e.g. the high complexity of the immune system and associated risk of setting up the wrong hypotheses, the increasing number of diseases in which the immune system is altered or involved, the increased requirements from regulatory authorities for pushing new immune therapies forward, the individual heterogeneity of the baseline status of the immune system, the differences in disease category and disease stage before treatment, the different effects of treatments in different patients with different treatment responses, the consequent need for treatment adaptation, whether increased dosages, decreased dosages or immediate treatment withdrawal, and/or the need for choosing the right combination of treatment in future trials.

The above is essential for all immune dysregulated conditions, and diseases with intervention via immune modulatory treatments, including: classical immune suppressive treatments (corticosteroids, cyclosporine, methotrexate, etc.); cellular treatments, such as gene therapy, stem cell transplantation, CAR T-cell treatment; check point inhibitors; the many different antibody treatments (with many different antibody effector functions); and vaccinations, whether against external agents (microorganisms, insect venoms, allergens, etc.) or tumor antigens.

In addition to treatment monitoring, also in the classical “wait-and-see” follow-up of individuals with immune dysregulated conditions immune monitoring will become progressively more important in order to predict outcome and to install, modify or withdraw treatment in an appropriate manner.

Many leukocyte/immune cell subsets have been identified in blood, but many other immune cell subsets are assumed to be exclusively or mainly detectable in tissues, where they have their specific tasks and effector functions. Nevertheless, the immune system has the unique capability of reaching the whole body via migration to all organs or organ systems. Such body-wide migration and homing of the immune cells can only be achieved via the blood stream and subsequently via the lymph system. This implies that the blood stream can be regarded as the highway of the immune system, allowing for trafficking throughout the total body, in search for targeted immune responses, scavenger tasks to keep the tissue debris free or body-wide scanning for inappropriate events such as tissue damage, microorganism invasion, or early neoplastic transformation and local tissue repair.

Consequently, in principle, all or virtually all immune cells or their precursors might be found in the blood stream, albeit that they might occur at low or very low frequencies and as unique maturation stages, which in part might be strongly dependent on the timing of the immune cell response. This implies that sufficient numbers of blood leukocytes need to be evaluated, most likely at least one million, up to 5 to 10 million or more at the appropriate time points.

All immune cells are derived from the hematopoietic stem cell compartment from where they differentiate into multiple lineages of immune cells with lymphoid and myeloid development being the two main pathways. The lymphoid cells form the basis of the adaptive immune system with the production of highly diverse antigen-specific receptors that allow highly specific recognition of many different antigens, i.e. the broad repertoire of many different immunoglobulin (Ig) molecules of B-cells and the broad repertoire of many different T-cell receptors (TcR) of T-cells. B-cells develop in the bone marrow from B-cell precursor cells to immature B-cells that arrive in the blood stream as immature B-cells and mostly naïve B-cells, which upon antigen-contact can further develop into Ig-secreting plasma cells and memory B-cells. The memory B-cells will respond efficiently and fast anywhere in the body, when the same antigen might be encountered again, with subsequent formation of extra memory B-cells and particularly, plasma cells for enhanced Ig production; this includes the production of Ig molecules of different classes, such as IgM, IgD, IgG, IgA, and IgE and the IgG and IgA subclasses. All related immature and naïve B-cell subsets, memory B-cell subsets, and plasmablast/plasma cell subsets will circulate through the body to reach their target tissues for generating efficient immune responses. Therefore these subsets are detectable in the blood stream, albeit that some B-cell subsets occur at relatively low frequencies of ≤10 cells per μL. When sufficient cells are being evaluated (preferably ≥1-5 million blood leukocytes), the immature and naïve B-cell subsets, the non-class-switched and class-switched memory B-cell subsets, and plasmablast/plasma cell subsets form a full B-cell pathway, all of which are detectable in blood.

T-cells develop in the thymus, where they stepwise differentiate into TcRyδ and TcRαβ T-cells with CD4+ and CD8+ TcRαβ T-cell subpopulations. These T-cell populations form the main T-cell populations in the blood stream. Already two decades ago, multiple maturation stages were discovered within the main T-cell populations from recent thymic emigrants and naïve T-cells to central memory T-cells, transitional memory T-cells, effector memory T-cells and terminal effector T-cells. In addition, also functional subsetting of CD4 T-helper (Th) cells and CD8 cytotoxic T-cells appeared to be possible.

Also the innate immune cells can be subdivided in different related lineages: granulocytic cells, such as the neutrophils, basophils and eosinophils; monocytes, such as the classical monocytes, intermediate monocytes, non-classical monocytes and FcERI+ monocytes; dendritic cells (DC), such as myeloid DC, plasmacytoid DC; NK-cells and other innate lymphoid cells, etc.

Whereas many proposals have been made available for flow cytometric blood leukocyte subsetting, the present inventors recognized that the following issues have not been possible thus far:

1: how to efficiently and simultaneously subset DC's in blood in a single measurement, also in relation to the various monocyte subsets and other myeloid cells;

2: how to subset CD4+ T-cells without complex intracellular cytokine profiles for defining CD4 T-helper subsets, follicular helper T (Tfh) cells, and regulatory T-cells (Tregs) also in relation to the many other T-cell subsets and maturation stages using a single antibody combination, preferably connected to subsetting of the various cytotoxic T-cells and NK cells.

3: how to differentiate maturation and functional subsetting within the plasmablast/plasma cell compartment also in relation to the various B-cell subsets and their IgH subclass expression.

The invention therefore aims at addressing one or more of the above issues, in particular by providing novel reagent compositions and methods that allow for cytometric blood leukocyte subsetting. Preferably, the reagents can be applied in classical multi-parameter flow cytometric technologies, using many different fluorochromes, currently increasing over 40 colors. Alternatively, metal-conjugated antibodies can be used for analysis in mass cytometry, currently increasing over 40 different “colors” (metals).

### SUMMARY OF THE INVENTION

In a first aspect, the invention provides a reagent composition for the cytometric immunophenotyping of leukocytes, comprising antibodies conjugated to a detectable label, the conjugated antibodies being directed against the following combination of markers: CD141, HLA-DR, CD16, CD33, CD300e, CD303 and CD14, wherein the antibodies directed against CD300e and CD303 may be conjugated to the same label. Such reagent composition is herein also referred to as a “DC-Monocyte tube”.

In a second aspect, the invention provides a reagent composition

A reagent composition for the cytometric immunophenotyping of leukocytes comprising antibodies conjugated to a detectable label, the conjugated antibodies being directed against the following combination of markers: CD27, CD45RA or CD45RO, CD62L, CD127, CD3, CD25, CCR10, CD183 (CXCR3), CD196 (CCR6). CD194 (CCR4), CD185 (CXCR5) and CD4. Such reagent composition is herein also referred to as a “CD4 T cell tube”.

In a third aspect, the invention provides a reagent composition a reagent composition for the cytometric immunophenotyping of leukocytes comprising antibodies conjugated to a detectable label, the conjugated antibodies being directed against the following combination of markers: CD20, CD38, CD62L, and CD138, optionally combined with CD19. Such reagent composition is herein also referred to as a ‘Plasma cell/B-cell tube’.

The invention also relates to reagent sets comprising two or more reagent compositions herein disclosed.

A further aspect relates to a diagnostic kit for cytometric immunophenotyping of leukocytes comprising one or more reagent composition(s) or sets of reagent compositions according to the invention, optionally together with instructions for use, buffer, and/or control samples.

Also provided is the use of a diagnostic kit according to the invention, for example in monitoring the effect of an immune modulatory treatment selected from the group consisting of classical immune suppressive treatments (corticosteroids, cyclosporine, methotrexate, etc.); cellular treatments, such as gene therapy, stem cell transplantation, CAR T-cell treatment; check point inhibitors; the many different antibody treatments (with many different antibody effector functions); and vaccinations, whether against external agents (microorganisms, insect venoms, allergens, etc.) or tumor antigens.

In a further embodiment, the invention provides a cytometric method for monitoring the immune status and/or the effect of an immune modulatory treatment of a subject, comprising the steps of:

(a) contacting one or more aliquots of a biological sample comprising leukocytes obtained from the subject with a reagent composition according to the invention;
(b) analyzing leukocytes in said aliquot(s) in a (flow or mass) cytometer; and
(c) storing and evaluating the data obtained.

### LEGENDS TO THE FIGURES

FIG. 1. Gating Strategy for DC-Monocyte Tube

This gating strategy is used for identification of 10 innate myeloid populations using antibodies directed against the “backbone” of 7 markers (HLA-DR, CD14, CD16, CD33, CD141, CD300e and CD303), the antibodies being conjugated to 6 distinct detectable labels. Panel A depicts the selection of singlets, further analyzed in the subsequent panels. Panels B-D show the strategy for identification of neutrophils and eosinophils, whereas panels E-M illustrate the gating approach for identification of the major monocytic populations: classical monocytes (cMo), intermediate monocytes (iMo) and non-classical monocytes (ncMo). Panels N-Q and R-V depict the analysis sequence for identification of myeloid dendritic cells (CD141⁺, CD1c⁺/CD14⁻ and CD1c⁺/CD14^{low}) and Axl+ and plasmacytoid dendritic cells, respectively. cMo, classical monocytes; iMo, intermediate monocytes; ncMo, non-classical monocytes; myDC, myeloid dendritic cell; pDC, plasmacytoid dendritic cell.

FIG. 2. Multidimensional representation (principal component analysis) of the distinct populations identified using the DC-monocyte tube for analysis of peripheral blood.

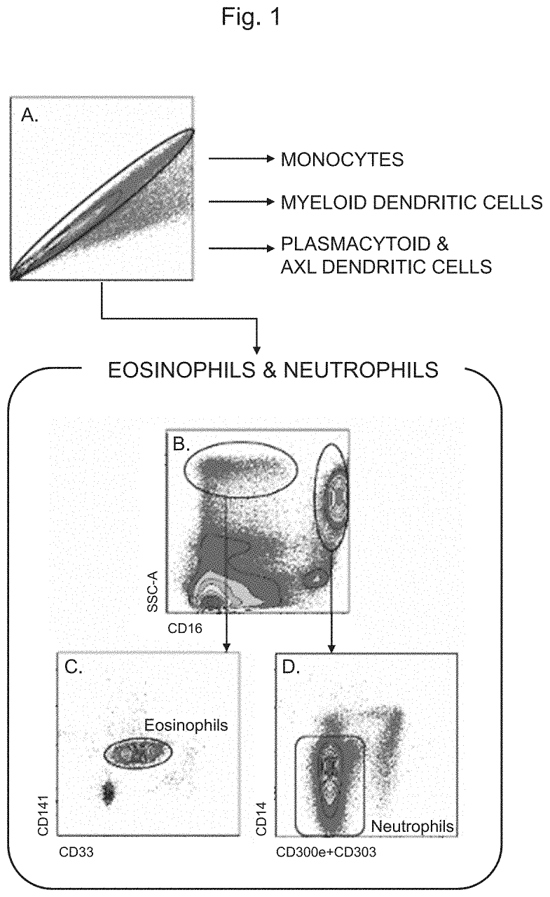

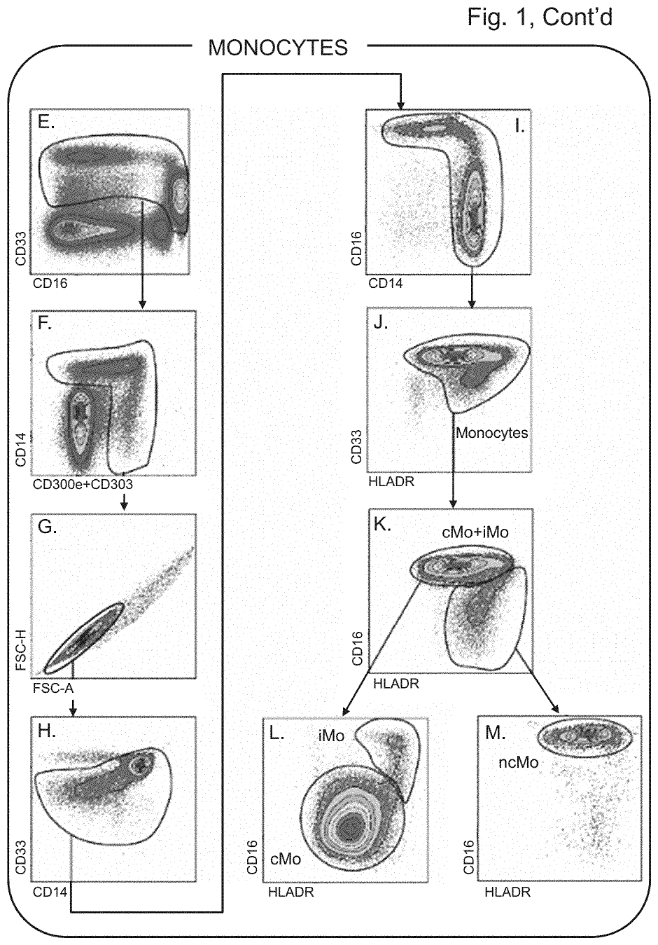

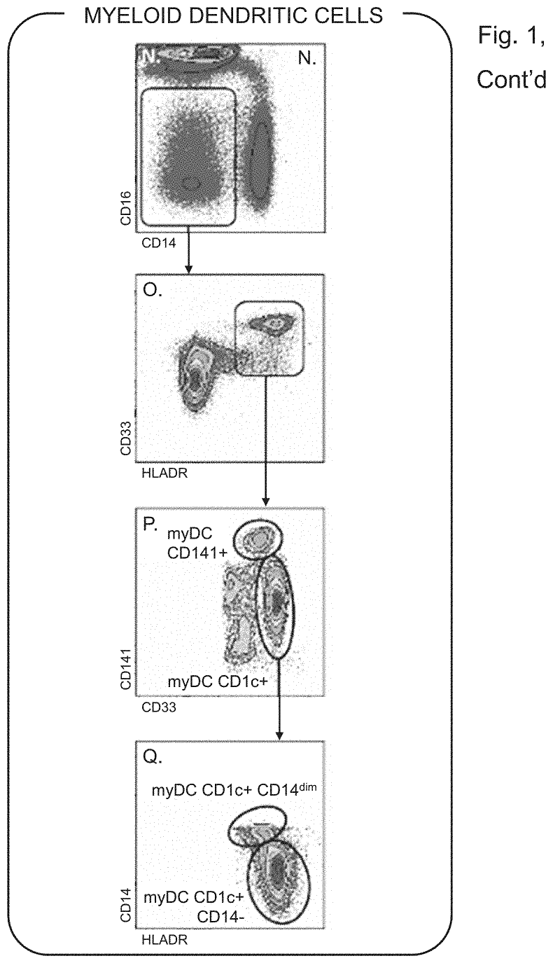

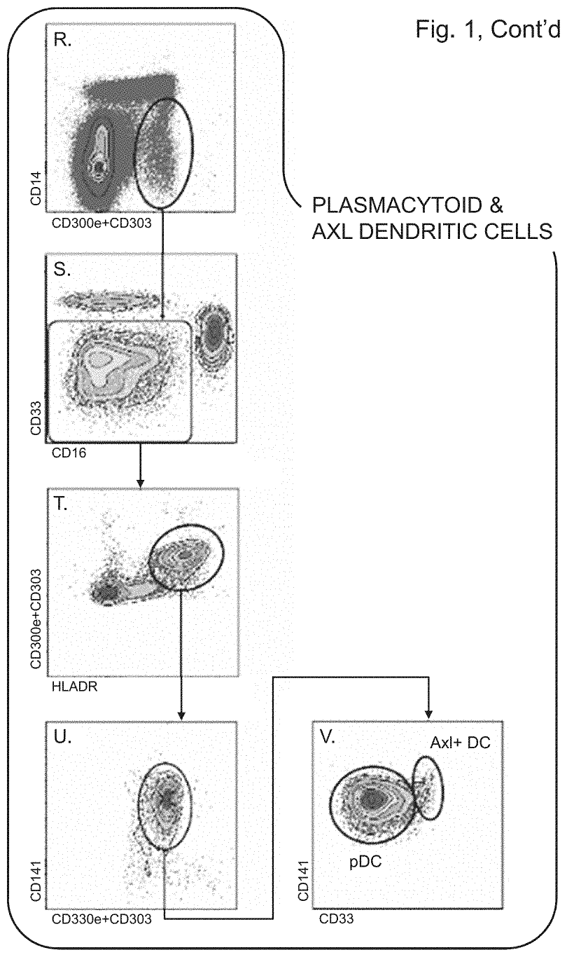

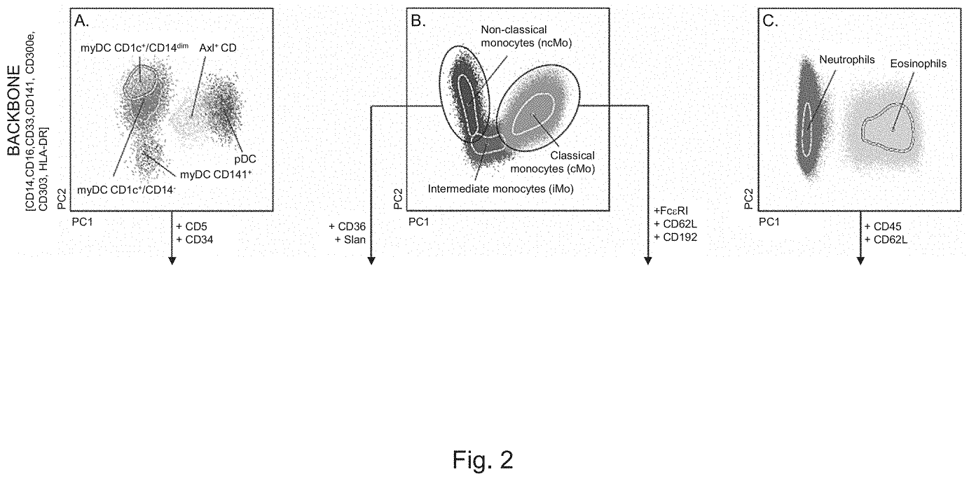

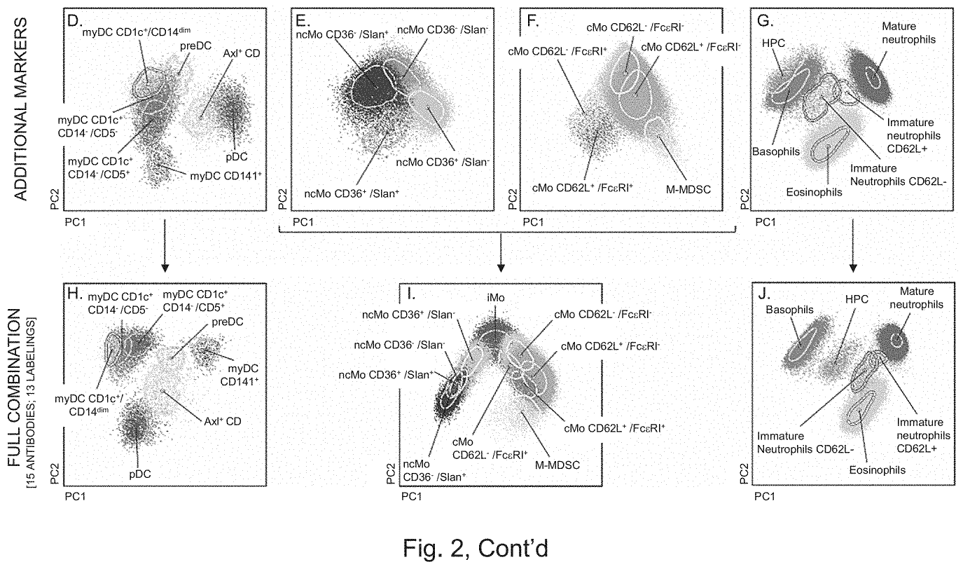

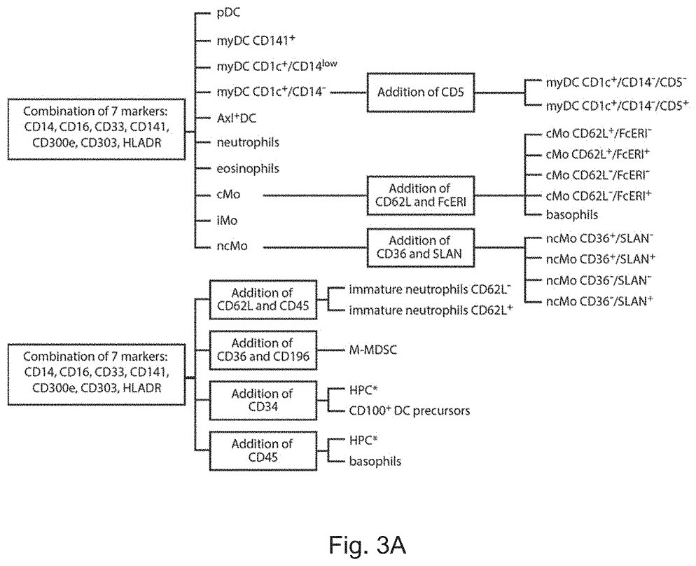

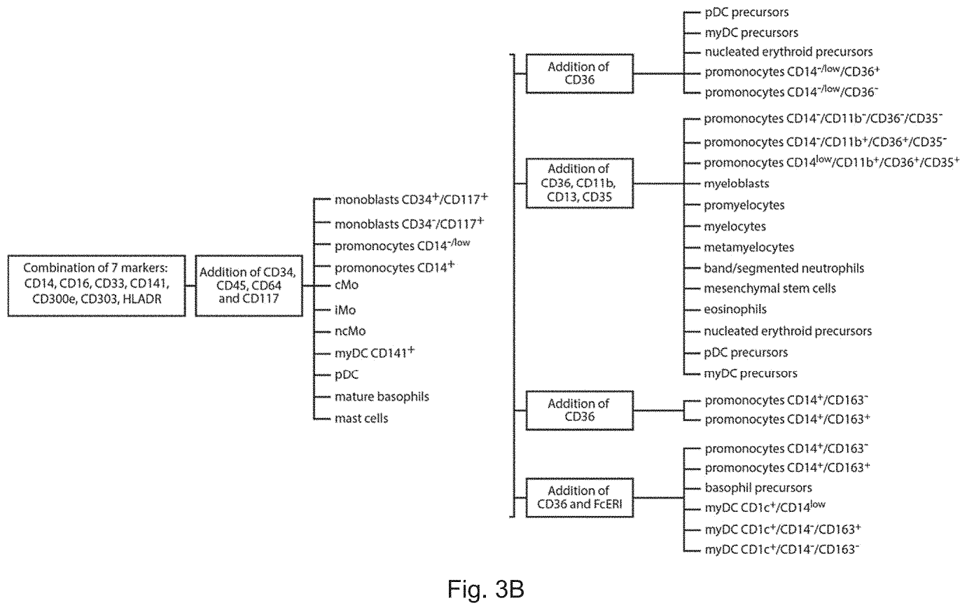

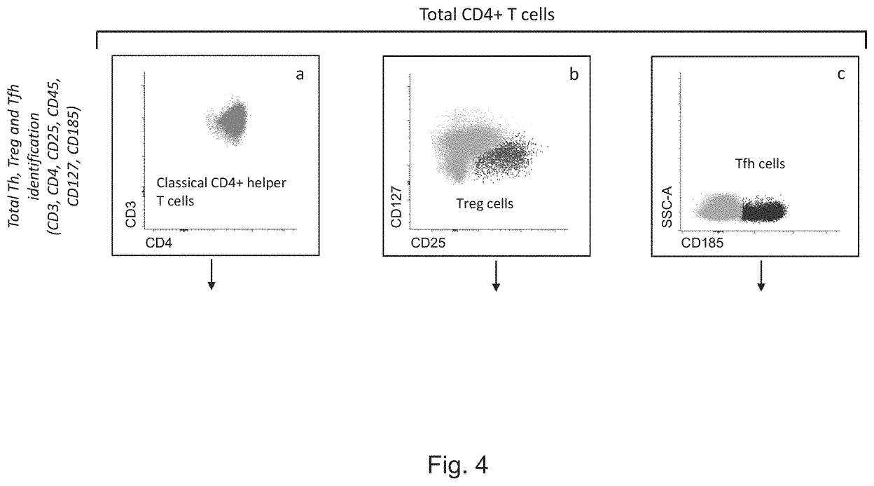

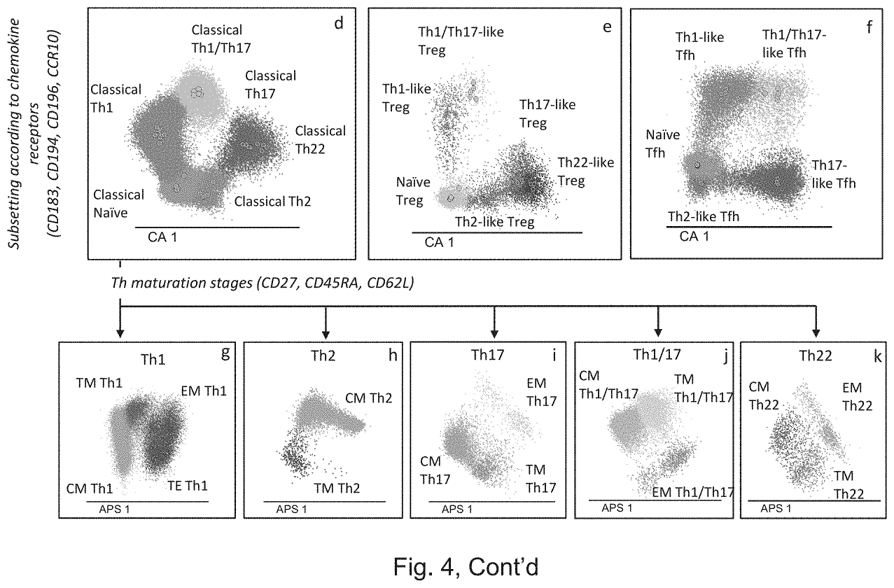

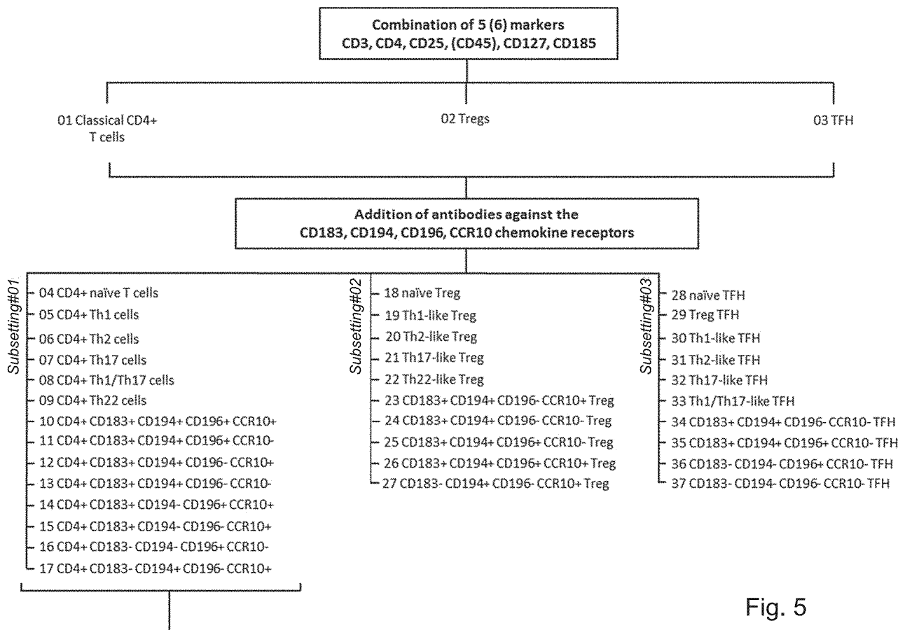

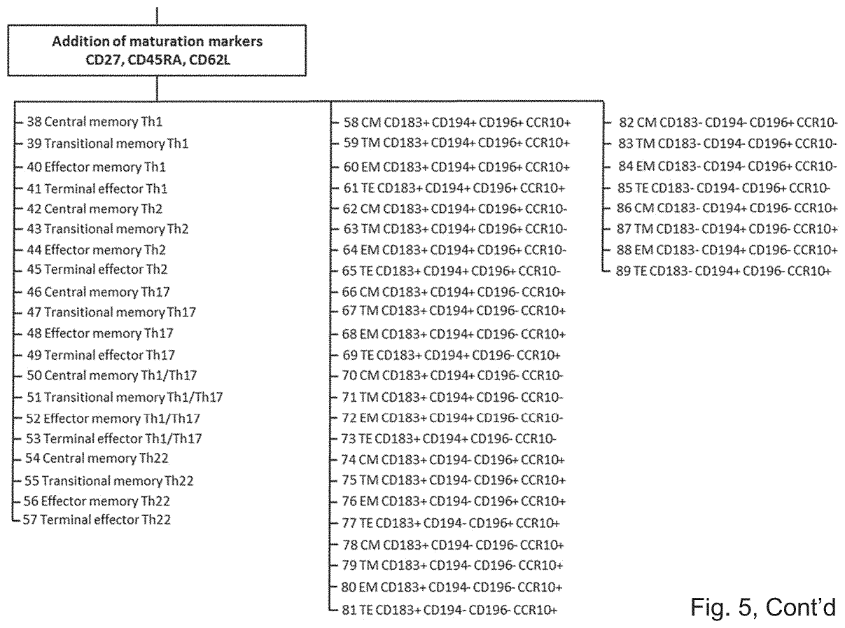

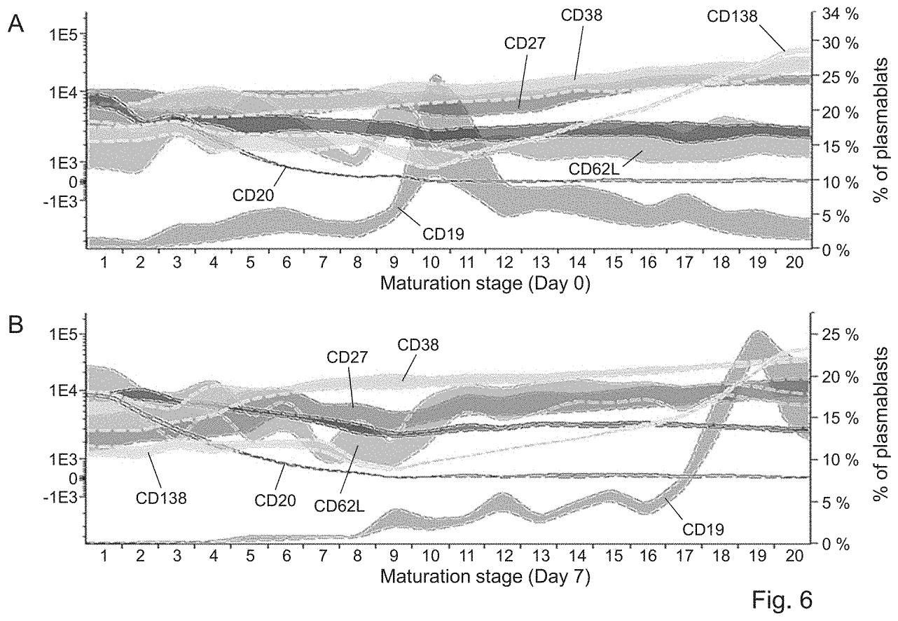

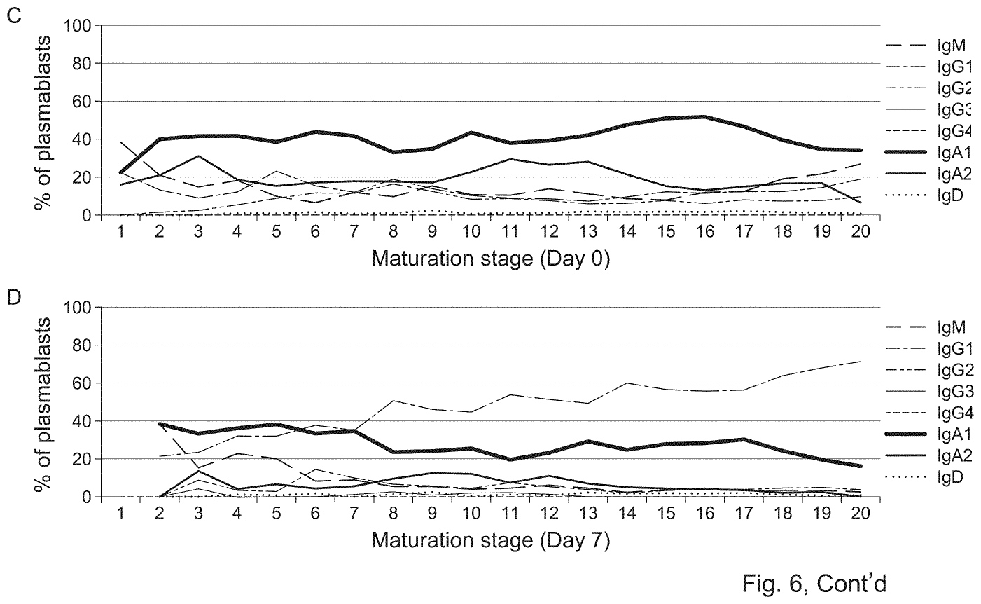

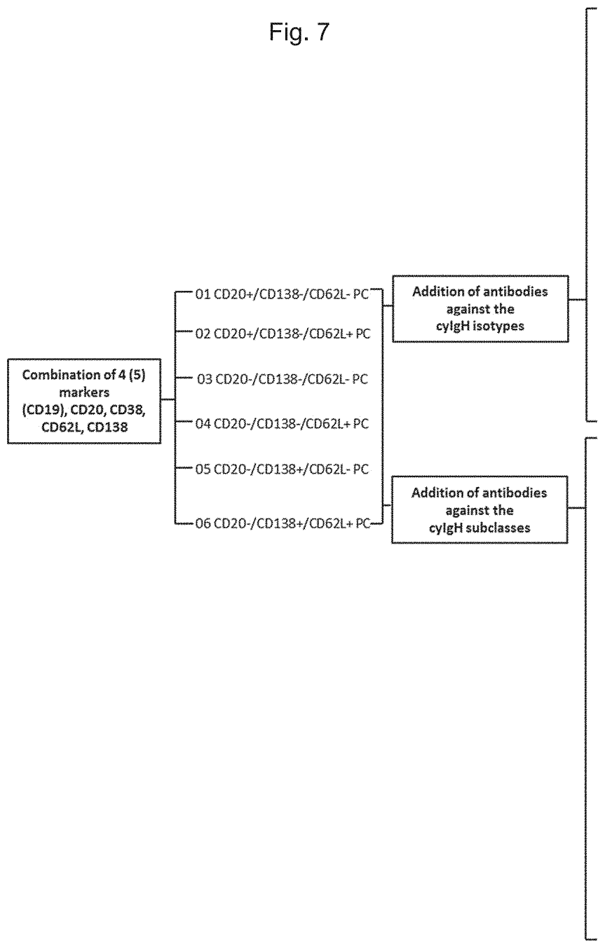

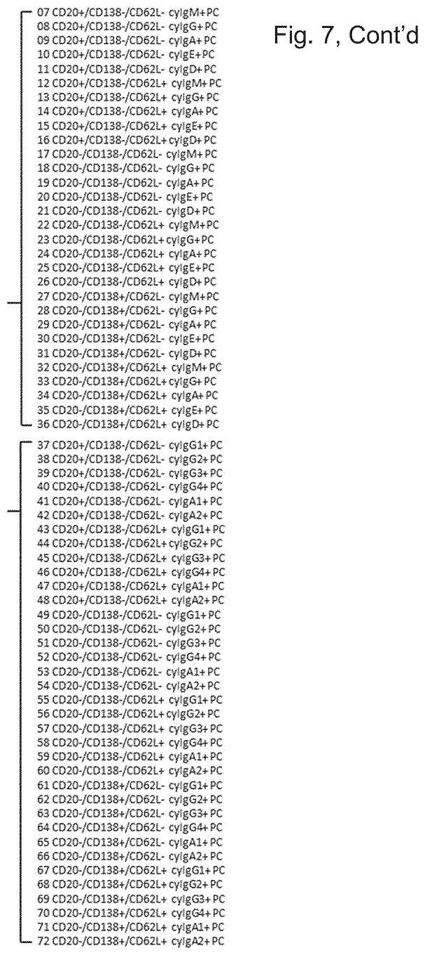

Panels A-C depict the 10 populations detected using the backbone of 7 markers conjugated with 6 labelings (see FIG. 1). Panel D shows the contribution of CD5 and CD34 for identification of pre-dendritic cells (DC) CD100+ and further subsetting of myeloid DC. Further subsetting of monocytic cells using CD36 and Sian and/or CD62L and FcERI is demonstrated in panels E and F, whereas the added value of CD45 and CD62L for identification basophils, immature neutrophils and hematopoietic precursor cells (HPC) is depicted in panel G. Panels H-J show the overall performance of the novel 15 antibody combination conjugated to 13 different detectable labels for the simultaneous identification of 23 distinct innate cell subsets.

PC, principal component; cMo, classical monocytes; iMo, intermediate monocytes; ncMo, non-classical monocytes; myDC, myeloid dendritic cell; pDC, plasmacytoid dendritic cell; HPC, hematopoietic precursor cell; M-MDSC, monocytic myeloid derived suppressor cell; preDC, pre-dendritic cell CD100+.

FIG. 3. Population tree of the DC-Monocyte tube.

Panel A. Population tree for blood analysis (14 markers) allows for detailed subsetting into 23 subsets.

Panel B. Population tree for bone marrow analysis (16 markers) allows for detailed subsetting into 19 bone marrow subsets.

*, Same population identified using different strategies.

cMo, classical monocytes; iMo, intermediate monocytes; ncMo, non-classical monocytes; myDC, myeloid dendritic cell; pDC, plasmacytoid dendritic cell; HPC, hematopoietic precursor cell; M-MDSC, monocytic myeloid derived suppressor cell.

FIG. 4. Major T-helper, Treg and Tfh populations identified with the CD4 T cell tube. Sequential strategy used for the identification of total classical helper CD4+ T cells (a), Tregs (b) and Tfh cells (c) using CD3, CD4, CD25, CD45, CD127 and CD185. Within each major population, different Th and Th-like subsets can be identified based on the expression of CD183, CD194, CD196 and CCR10, as shown in panels d, e and f for classical Th cells, Tregs and Tfh cells, respectively, in which canonical multivariate analysis (CA) was performed. Finally, within each Th subset, distinct maturation stages are identified based on their expression profile for CD27, CD45RA and CD62L, as displayed in two-dimensional automatic population separator (APS) views—principal component (PC) 1 vs PC2—in panels g-k for classical Th cells.

Th, helper T cells; Tregs, regulatory T cells; Tfh, follicular helper T cells.

FIG. 5. Population tree of CD4 T-cell tube.

FIG. 6. Immunophenotypic patterns of circulating plasmablasts and plasma cells in peripheral blood with the antibody combination for identification and characterization of plasmablasts and plasma cells. Dissection of the distinct maturation patterns of circulating plasmablasts and plasma cells combining antibodies against the markers CD19, CD20, CD38, CD62L and CD138 is represented on maturation diagrams in a healthy individual before vaccination (panel A) and seven days after vaccination (panel B). Color lines represent the level of expression of markers corresponding to each maturation stage. Grey lines represent the percentage of events within each maturation stage. Twenty maturation stages were defined by default for smooth graphical representation along the maturation pathway of each cell lineage. Dissection of 20 plasmablast/plasma cell maturation stages according to the expression of IgH classes and subclasses is represented in healthy individuals before vaccination (panel C) and seven days after vaccination (panel D). Color lines represent the percentage of plasmablasts/plasma cells expressing each IgH class and subclass from total plasmablasts/plasma cells per maturation stage.

FIG. 7. Population tree of Plasmablast/plasma cell & B-cell tube

### DETAILED DESCRIPTION

The invention relates in some embodiments to improved and advantageous reagent compositions and kits comprising the same. The term “reagent composition” (or “cytometric panel”) as used herein relates to a cocktail of antibodies (or other specific antigen-binding agents) which are conjugated to (directly or indirectly) a detectable label.

Antibodies, or immunoglobulins, comprise two heavy chains linked together by disulfide bonds and two light chains, each light chain being linked to a respective heavy chain by disulfide bonds in a “Y” shaped configuration. The variable domains of each pair of light and heavy chains form the antigen binding site. The isotype of the heavy chain (gamma, alpha, delta, epsilon or mu) determines immunoglobulin class (IgG, IgA, IgD, IgE or IgM, respectively). It should be understood that when the terms “antibody” or “antibodies” are used, this is intended to include intact antibodies, such as polyclonal antibodies or monoclonal antibodies (mAbs), as well as proteolytic fragments thereof such as the Fab or F(ab′)2 fragments. Further included within the scope of the invention are chimeric antibodies; recombinant and engineered antibodies, and fragments thereof, as well as other molecules comprising at least an antigen binding site (retaining the antigen binding capacity) of an antibody. In a preferred embodiment, the composition comprises a panel of conjugated monoclonal antibodies. (Monoclonal) antibodies against the indicated markers can be commercially obtained from various companies, including Becton/Dickinson (BD) Biosciences, Biolegend, Dako, Beckman Coulter, CYTOGNOS, Caltag, Myltenyi, Pharmingen, Exbio, Sanquin, Invitrogen, and the like.

Antibodies for use in the present invention are conjugated to a label that is detectable by cytometry, e.g. by flow or mass cytometry. Suitable types of detectable labels include fluorochromes (fluorophores), quantum dots, and metal-isotope labels. As will be understood, to facilitate simultaneous detection, all antibodies present in a reagent composition of the invention are conjugated to the same type of detectable label, e.g. all are fluorochrome conjugated or all are isotope labeled. In addition, flow cytometry uses the light properties scattered from cells or particles for identification or quantitative measurement of physical properties.

In one embodiment, the antibody is conjugated to a fluorochrome. A fluorochrome (or “fluorophore”) is a chemical which can absorb energy from an excitation source (laser beam) and emit photons at a longer wavelength (fluorescence), which is captured by optical detectors of the flow cytometer. Suitable fluorochromes for conjugating antibodies are known in the art. Each fluorophore has a characteristic peak excitation and emission wavelength, and the emission spectra often overlap. Consequently, the combination of labels which can be used depends on the wavelength of the lamp(s) or laser(s) used to excite the fluorochromes and on the detectors available. The fluorochromes are preferably selected for brightness, limited spectral overlap and limited need for compensation, stability, etc.

A wide range of fluorophores can be used as labels in flow cytometry. Fluorochromes of particular use in a reagent composition according to the invention include those of the Brilliant Violet (BV) series, such as BV421 or functional equivalent thereof, BV510 or functional equivalent thereof, BV605 or functional equivalent thereof, BV650 or functional equivalent thereof, BV711 or functional equivalent thereof. BV786 or functional equivalent thereof; fluorescein isothiocyanate (FITC) or functional equivalent thereof (e.g. BB515), PerCP Cy5.5 or functional equivalent thereof, phycoerythrin (PE) or functional equivalent thereof, phycoerythrin/CF594 (PE CF594) or functional equivalent thereof, phycoerythrin/cyanine5 (PE-Cy5) or functional equivalent thereof phycoerythrin/cyanine7 (PE-Cy7) or functional equivalent thereof, allophycocyanine (APC) or functional equivalent thereof (Alexa647), AF700 or functional equivalent thereof, and allophycocyanine/H7 (APC-H7) or functional equivalent thereof.

The following panel of fluorochromes is of particular use in a 12-color reagent composition according to the invention: (1) BV421 or functional equivalent thereof, (2) BV510 or functional equivalent thereof, (3) BV605 or functional equivalent thereof; (4) BV711 or functional equivalent thereof, (5) BV786 or functional equivalent thereof, (6) fluorescein isothiocyanate (FITC) or functional equivalent thereof (e.g. BB515), (7) PerCP Cy5.5 or functional equivalent thereof, (8) phycoerythrin (PE) or functional equivalent thereof (9) phycoerythrin/cyanine7 (PE-Cy7) or functional equivalent thereof, (10) allophycocyanine (APC) or functional equivalent thereof (Alexa647), (11) AF700 or functional equivalent thereof, e.g. APC-R700, (12) allophycocyanine/H7 (APC-H7) or functional equivalent thereof, e.g. APC-Cy7.

The following panel of fluorochromes is of particular use in a 14-color reagent composition according to the invention: (1) BV421 or functional equivalent thereof, (2) BV510 or functional equivalent thereof, (3) BV605 or functional equivalent thereof; (4) BV650 or functional equivalent thereof, (5) BV711 or functional equivalent thereof, (6) BV786 or functional equivalent thereof, (7) fluorescein isothiocyanate (FITC) or functional equivalent thereof (BB515), (8) PerCP Cy5.5 or functional equivalent thereof, (9) phycoerythrin (PE) or functional equivalent thereof, (10) phycoerythrin/CF594 (PE CF594) or functional equivalent thereof, (11) phycoerythrin/cyanine5 (PE-Cy5) or functional equivalent thereof (12) phycoerythrin/cyanine7 (PE-Cy7) or functional equivalent thereof, (13) allophycocyanine (APC) or functional equivalent thereof (Alexa647), (14) AF700 or functional equivalent thereof, (15) allophycocyanine/H7 (APC-H7) or functional equivalent thereof.

In another embodiment, the antibody is conjugated to a quantum dot. Quantum dots are sometimes used in place of traditional fluorophores because of their narrower emission peaks.

In yet another embodiment, the antibody is conjugated to a metal isotope, allowing for detection by mass cytometry, also called cytometry by time-of-flight (CyTOF). Advances in single-cell mass cytometry have increasingly improved highly multidimensional characterization of immune cell heterogeneity. The immunoassay multiplexing capacity relies on monoclonal antibodies labeled with stable heavy-metal isotopes.

Mass cytometry overcomes the fluorescent labeling limit by utilizing lanthanide isotopes attached to antibodies. This method could theoretically allow the use of 40 to 60 distinguishable labels or more. Mass cytometry is fundamentally different from flow cytometry: cells are introduced into a plasma, ionized, and associated isotopes are quantified via time-of-flight mass spectrometry. Although this method permits the use of a large number of labels, it currently has lower throughput capacity than flow cytometry. It also destroys the analysed cells, precluding their recovery by sorting. Finally, a major fraction of cells is lost before reaching the cone for measurement.

To date, a variety of rare-earth elements and noble and post-transition metal isotopes have been used in mass cytometry. For example, Han et al. (2018, Nature Protocols volume 13, pages 2121-2148) disclose protocols for conjugating monoclonal IgG antibodies with 48 high-purity heavy-metal isotopes: (i) 38 isotopes of lanthanides, 2 isotopes of indium, and 1 isotope of yttrium; (ii) 6 isotopes of palladium; and (iii) 1 isotope of bismuth.

In one embodiment, a reagent composition of the invention comprises a panel of antibodies that are conjugated to metals of different mass, in particular isotopes of metals, preferably isotopes of metals selected from the group consisting of Pr, Bi, Y, Nd, Sm, Eu, Gd, Tb, Dy, Ho, Er, Tm, Yb, Lu, Rh, Cd, In, Ir, Pt and Pd. Specific examples include 103Rh, 106Cd, 110Cd, 111Cd, 112Cd, 113Cd, 114Cd, 115In, 116Cd, 141Pr, 142Nd, 143Nd, 144Nd, 145Nd, 146Nd, 147Sm, 148Nd, 149Sm, 150Nd, 151Eu, 152Sm, 153Eu, 154Sm, 155Gd, 156Gd, 158Gd, 159Tb, 160Gd, 161Dy, 162Dy, 163Dy, 164Dy, 165Ho 166Er, 167Er, 168Er, 169Tm, 170Er, 171Yb, 172Yb, 173Yb, 174Yb, 175Lu, 176Yb, 191Ir, 193Ir, 194Pt, 198Pt and 209Bi.

In a typical process of sample preparation for mass cytometric analysis of cells, cells are incubated (or “stained”) with a panel of metal-tagged antibodies that target antigens (markers) of interest. A DNA intercalator can be incorporated into the panel to allow determination of nucleated cells from nonnucleated cells. Cells are stained under resting or stimulating conditions and are fixed prior to analysis. The samples are washed to remove unbound antibody and salts and diluted to an appropriate cell concentration. Cells are then passed in a single-cell suspension into a nebulizer, which aerosolizes the cells into droplets for introduction into the mass cytometer. Upon entering the instrument, cells travel through an argon plasma at 7000° K which completely vaporizes and ionizes the cell and the attached antibodies into a cloud of single-atom ions. The size of the cloud is largely driven by gas expansion kinetics and is relatively independent of the cell size. The ion cloud is filtered by a quadrupole to remove common biological elements with a mass less than ˜75 Da, to leave only the heavy metal ions that were attached to the staining antibodies directed against the marker set of interest. The ions within the cloud are separated by their mass-to-charge ratio in a time-of-flight (TOF) mass spectrometer. Ion signals are integrated on a per-cell basis, resulting in single-cell measurements for analysis.

The panel of antibodies in a reagent composition provided herein may be used in some embodiments to simultaneously label a cell-containing biological sample (in suspension). In other words, the sample is typically and advantageously incubated with an entire cytometric panel as disclosed herein, thus allowing characterization of multiple cell populations in a single measurement step, using multi-parametric analysis (“multiplexing”) of the antigen co-expression pattern on single cells from the sample. Accordingly, antibodies that are directed to distinct cellular targets (“markers”) for which separation is desired within a panel, are labeled with distinct (non-equivalent) detectable labels, and are referred to herein as distinct conjugated antibodies. The marker can be a protein that is expressed on a cell's surface (cell surface marker) or in the cytoplasm (cytoplasmic marker; cy marker). Typically, the markers of the present invention are human markers. “CD” stands for cluster designation and is a nomenclature for the identification of specific (human) cell surface antigens defined by monoclonal antibodies.

In some embodiments, a reagent composition comprises antibodies against different markers, but wherein a subset, e.g. two or three, of these antibodies are conjugated to the same detectable label. See for example the reagents of Table 1, comprising antibodies against CD300e and CD303, each conjugated to the same detectable label. The reagent composition may comprise two or more of such pairs of antibodies, wherein the antibody within either one of the pairs is conjugated to the same detectable label, but wherein between different pairs the labels are distinguishable. For example, both antibodies of the first pair are conjugated to fluorochrome A and both antibodies of the second pair are conjugated to fluorochrome B. Thus, within each pair the fluorochromes are the same. See for example Table 1, disclosing reagents comprising antibodies against SLAN and FcERI, each conjugated to fluorochrome “F9”, and antibodies against CD300e and CD303, each conjugated to fluorochrome “F12”.

It is also possible that the reagent composition comprises multiple conjugated antibodies directed against the same target (marker), each antibody being conjugated to a distinct detectable label. See for example Table 7, disclosing reagents comprising two antibodies against IgA1, the first being conjugated to fluorochrome “F8”, and the second to fluorochrome “F12”; two antibodies against IgG2, the first being conjugated to fluorochrome “F7”, and the second to fluorochrome “F9”, and two antibodies against IgD, the first being conjugated to fluorochrome “7”, and the second to fluorochrome “F12”.

The term “kit” as used herein relates to an article of manufacture comprising at least one and typically a plurality of reagent compositions of the invention, and optionally additional reagents, e.g. reagents for cell dissociation, purification, permeabilization, control samples or antibodies, or other reagents for use in flow cytometry. The kit may further contain instructions for using the at least one reagent composition in the methods of the invention, e.g. instructions for analyzing the results measured using multi-parametric analysis as detailed herein. In one embodiment, the invention provides a 12-color diagnostic kit, comprising one or more 12-color reagent compositions herein disclosed.

A cytometric method for monitoring the immune status and/or the effect of an immune modulatory treatment of a subject typically comprises the steps of: (a) contacting an aliquot of a biological sample comprising leukocytes obtained from the subject with a reagent composition as herein disclosed; (b) analyzing leukocytes in said aliquot in, depending on the type of detectable label, a flow or mass cytometer; and (c) storing and evaluating the data obtained.

Any type of sample known or suspected to contain leukocytes may be used directly, or after lysing non-nucleated red cells, or after density centrifugation, or after cell sorting procedures. For example, the sample is peripheral blood, bone marrow, tissue sample such as lymph nodes, adenoid, spleen, or liver, or other type of body fluid such as cerebrospinal fluid, vitreous fluid, synovial fluid, pleural effusions or ascites. Peripheral blood (PB) or bone marrow (BM) is preferred.

Preferably, step (c) of a method provided herein comprises combining the immunophenotypic information of two or more selected cell populations from multiple tubes according to the so-called nearest neighbor calculations in which individual cells from one aliquot of a sample are matched with corresponding individual cells from another aliquot of the same sample, according to their markers and scatter profile. Advantageously, the method comprises the use of software for data integration and multidimensional analysis of flow cytometry files, preferably wherein said software is INFINICYT™.

In the following sections A through E, specific preferred embodiments of the invention are presented. Any of these embodiments can be used on its own, or in combination with each other. Whereas the embodiments shown relate to antibodies that are conjugated to a fluorochrome as detectable label, it will be understood and appreciated that variant embodiments involving other types of detectable labels, e.g. metal-isotopes, are also encompassed. As used herein, the expression “marker combination” discussed in relation to a reagent composition implies that the composition comprises conjugated antibodies directed at each marker of said marker combination.

### A. DC-Monocyte Tubes for Blood and BM Studies

Since the first discovery of dendritic cells (DC), multiple subsets have stepwise been identified. While some DC subsets have been found exclusively in tissues, others have also been found in blood. These blood DC include the classically defined plasmacytoid DC (pDC) and myeloid DC (myDC), as well as their subsets, myDC CD1c⁺/CD14^{low}, myDC CD1c⁺/CD14⁻/CD5⁻, myDC CD1c⁺/CD14⁻/CD5⁺ and myDC CD141+, in addition to the recently described Axl⁺ DC and CD100⁺ DC precursors (pre-DC) (Villani, Satija et al. 2017, Yin, Yu et al. 2017, Collin and Bigley 2018). For some dendritic cell populations, several alternative markers can be used for their accurate identification, such as CD123^{high} CD303 and CD304 for identification of pDC, CD11c and CD33 for myDC and CD141 or CLEC9A for CD141⁺ myDC (Bachem, Guttler et al. 2010, Heinze, Elze et al. 2013, Fromm, Kupresanin et al. 2016, Alcantara-Hernandez, Leylek et al. 2017, See, Dutertre et al. 2017, Villani, Satija et al. 2017, Collin and Bigley 2018). In contrast, other subsets are defined in the literature based on single markers, e.g. Axl⁺ DC are identified based on Axl expression (also in combination with SIGLEC6), CD100⁺ myDC precursors based on CD100 in combination with CD34, CD1c⁺ myDC based on CD1c (Villani, Satija et al. 2017, Collin and Bigley 2018).

Even though several attempts have been made to design flow cytometry panels for identification of DC populations (Autissier, Soulas et al. 2010, Hasan, Beitz et al. 2015, Fromm, Kupresanin et al. 2016, Draxler, Madondo et al. 2017), until now nobody has taught or suggested how to identify all the above DC populations (including the newly described subsets) simultaneously in blood with a limited number of markers in a single-tube approach, in addition to other innate cells such as monocyte subsets, granulocyte subsets and myeloid derived suppressor cells (MDSC).

The present inventors solved this problem by providing a reagent composition for the cytometric immunophenotyping of leukocytes, comprising antibodies conjugated to a detectable label, the conjugated antibodies being directed against the following combination of markers: CD141, HLA-DR, CD16, CD33, CD300e, CD303 and CD14, wherein the antibodies directed against CD300e and CD303 may be conjugated to the same label.

### A.1 DC-Monocyte Tubes for Blood Analysis

The novel DC/monocyte reagent comprises conjugated antibodies against an innovative panel of 7 “backbone” markers (CD14. CD16, CD33, CD141. CD300e. CD303. HLX-DR) conjugated to 6 or 7 distinct labels (CD300e and CD303 can be combined in the same labeling; Table 1, tube BB1). In this 7-marker combination, the marker CD300e is critically important because its expression profile distinguishes between monocytes and distinct DC (positive/high versus negative/low, respectively). Antibodies against the markers CD141 and CD303 are essential for recognition of the CD141+ myDC and pDC, respectively. Herewith, the antibody panel of the invention provides unexpected advantages over reagent compositions known in the art, including WO2017/094008 disclosing a cytometric panel comprising antibodies against, among others, CD33, HLA-DR, CD14, CD16, CD303 and CD141, yet lacking the essential anti-CD300e antibody.

In one embodiment, for reasons of efficient usage of labels, the antibodies directed against CD300e and CD303 in an antibody panel of the invention are conjugated to the same detectable label. This is possible because the CD300e and CD303 markers are mutually exclusive markers, i.e. they are not present on the same monocyte and DC subsets. CD300e is expressed on all blood monocytic subsets at a greater intensity than on some DC subsets where CD300e is negative or dimly expressed, whereas CD303 is typically expressed on pDC and Axl+DC.

On its own, the 7 marker backbone reagent composition provided herein allows already for the identification of 5 subsets of DC (FIG. 1; FIG. 2A), and 3 monocyte subsets (FIG. 1; FIG. 2B) in addition to neutrophils and eosinophils (FIG. 1; FIG. 2C) for a total of 10 innate cell subsets including (FIG. 1 with full gating strategy):
pDC: HLA-DR⁺⁺. CD14⁻, CD16⁻, CD33^{-/low}, CD141⁺, CD300e⁻, CD303⁺, SSC^{low}
myDC CD141+: HLA-DR⁺⁺, CD14⁻, CD16⁻, CD33⁺⁺, CD141⁺⁺, CD300e⁻, CD303⁻, SSC^{low}
myDC CD1c+ CD14−: HLA-DR⁺⁺, CD14⁻, CD16⁻, CD33⁺⁺, CD141^{low}, CD300e^{-/low}. CD303⁻, SSC^{low}
myDC CD1c+ CD14low: HLA-DR⁺⁺, CD14^{low}, CD16⁻, CD33⁺⁺. CD141^{low}, CD300e^{-/low}, CD303⁻, SSC^{low}
Axl+DC: HLA-DR⁺⁺, CD14⁻, CD16⁻, CD33⁺, CD141⁺, CD300e⁻, CD303^{low+}, SSC^{low}
Classical monocytes (cMo): HLA-DR⁺, CD14⁺, CD16⁻, CD33⁺⁺, CD141^{low}, CD300e⁺, CD303⁻, SSC^{int}
Intermediate monocytes (iMo): HLA-DR⁺⁺, CD14⁺, CD16⁺, CD33⁺⁺, CD141^{low}, CD300e⁺⁺, CD303⁻, SSC^{int}
Nonclassical monocytes (ncMo): HLA-DR⁺, CD14^{-/low}, CD16⁺, CD33⁺, CD141^{low}, CD300e⁺⁺, CD303⁻, SSC^{int}
Neutrophils: HLA-DR⁻, CD14⁻, CD16⁺⁺, CD33⁺, CD141⁻, CD300e⁻, CD303⁻, SSC^{high}
Eosinophils: HLA-DR⁻, CD14⁻, CD16⁻, CD33^{low}, CD141⁻, CD300e⁻, CD303⁻, SSC^{high}

Addition of an antibody against CD5 to the above backbone combination (for a total of 8 markers combined with 7 labelings; Table 1, Tube BB2A) allows for further subsetting of the CD1c⁺ myDC into CD5⁻ and CD5⁺ subsets (FIG. 2D; FIG. 3A). Similarly, addition of CD34 to the backbone combination of 7 markers combined with 6 labelings (Table 1, tube BB2B), provides clear cut identification of hematopoietic precursors (HLA-DR^{+/++}, CD14⁻, CD16⁻, CD33⁺, CD141^{-/+}, CD300⁻, CD303^{-/+}, CD34^{low/+}, SSC^{low}) as well as CD100+ DC precursors (preDC, defined as HLA-DR⁺⁺, CD14⁻, CD16⁻, CD33⁺, CD141⁻, CD300e⁻, CD303⁻, CD34^{low}, SSC^{low} (FIG. 2D; FIG. 3A). Simultaneous addition of antibodies against CD5 and CD34 to the above backbone in the absence of CD45, would allow for the discrimination between myDC CD1c⁺/CD14⁻/CD5⁻ from myDC CD1c⁺/CD14⁻/CD5⁺, as well as of CD100⁺ DC precursors and CD34+ hematopoietic precursor cells, and therefore for the identification of a total of 13 cell subsets (FIG. 2D; FIG. 3A). Addition of an antibody against CD45 will further allow identification of basophils (HLA-DR⁻, CD14⁻, CD16^{-/+}, CD33⁺, CD141⁻, CD300e⁻, CD303⁻, CD34⁻, CD5⁻, CD45^{low}, SSC^{low}) (14 innate cell subsets).

For further subsetting of monocytes, the CD36 plus SLAN and/or CD62L plus FcERI pairs of markers can be added to any to the above combinations for further identification of subsets of cMo and ncMo, including ncMo CD36⁺/SLAN⁻, ncMo CD36⁺/SLAN⁺, ncMo CD36⁻/SLAN⁻, ncMo CD36⁻/SLAN⁺ (FIG. 2E; FIG. 3A), as well as cMo CD62L⁺/FcERI⁻, cMo CD62L⁺/FcERI⁺, cMo CD62L⁻/FcERI⁻, cMo CD62L⁻/FcERI⁺ (FIG. 2F; FIG. 3A). When these latter 4 markers are added together, antibodies against SLAN and FcERI can be both conjugated to the same detectable label, while antibodies against CD62L and CD36 are conjugated each with a different labeling (see Table 1). Of note, the addition of FcERI and CD62L antibodies to the above mentioned backbone antibodies also allows for further identification of basophils (HLA-DR⁻, CD14⁻, CD16^{-/+}, CD33⁺, CD141⁻, CD300e⁻, CD303⁻, CD62L^{high}, FcεRI⁺, SSC^{low}) (Table 1, tube BB3B; FIG. 3A).

In another embodiment, only an anti-CD45 antibody is added to the above backbone combination of 7 markers, which would further allow identification of basophils (HLA-DR⁻, CD14⁻, CD16^{-/+}, CD33⁺, CD141⁻, CD300e⁻, CD303⁻, CD45^{low}, SSC^{low}; Table 1, tube BB2C) and hematopoietic precursors in blood (HLA-DR+/++, CD14⁻, CD16⁻, CD33⁺, CD141^{-/+}, CD300e⁻, CD303⁻, CD45^{low}, SSC^{low}) for a total of 12 innate cell populations (FIG. 2G; FIG. 3A).

Addition to either the backbone of 7 markers combined with 6 or 7 labelings or to any of the above combination of markers, of CD36 plus CD192 and/or CD62L plus CD45 antibodies would allow identification of monocytic (M)-MDSC (FIG. 2F; FIG. 3A), and/or two populations of immature neutrophils [phenotypically compatible with polymorphonuclear (PMN)-MDSC] (FIG. 2G; FIG. 3A), respectively:
M-MDSC: HLA-DR⁻, CD14⁻, CD16⁻, CD33⁺⁺, CD36^{-/low}, CD45⁺, CD62L⁺, CD141⁻, CD192(CCR2)^{-/low}, CD300e⁻, CD303⁻, SSC^{int}
Immature neutrophils CD62L−: HLA-DR⁻, CD14⁻, CD16⁻, CD33⁺⁺, CD36⁻, CD45^{low}, CD62L−, CD141⁻, CD192(CCR2)⁻, CD300e⁻, CD303+, SSC^{hi}
Immature neutrophils CD62L+: HLA-DR⁻, CD14⁻, CD16^{low}, CD33⁺⁺, CD36⁻, CD45^{low}, CD62L⁺, CD141⁻, CD192(CCR2)⁻, CD300e⁻, SSC^{hi}

The 15 antibody combination proposed here (Table 1A) allows simultaneous identification of at least 23 distinct innate cell subsets in 1 mL of blood, including pDC, myDC CD1c⁺/CD14^{low}, myDC CD1c⁺/CD14−/CD5⁻, myDC CD1c/CD14−/CD5+, myDC CD141+, Axl⁺ DC, pre-DC (CD100+), four subsets of classical monocytes (cMo) [CD62L+/FcεRI⁻, CD62L+/FcεRI⁺, CD62L⁻/FcεRI⁻ and CD62L⁻/FcεRI⁺], intermediate monocytes (iMo), four populations of non-classical monocytes (ncMo) [CD36+/Slan⁻, CD36−/Slan⁻, CD36+/Slan+. CD36−/Slan+], as well as basophils, three populations of neutrophils (mature neutrophils, immature neutrophils CD62L⁻ and CD62L⁺), eosinophils, monocytic myeloid-derived suppressor cells (M-MDSC) and hematopoietic precursor cells (HPC) (FIGS. 1 and 2; FIG. 3A). Adding of the CD1c, Axl and/or CD100 antibodies as 16^{th}, 17^{th} and/or 18^{th} antibody will further confirm the above innate cell subset definition (Table 1A). If antibodies against all three markers are added, the CD14 and CD34 antibodies can be conjugated to the same label, because these two markers will not be co-expressed on the same cell in the DC-monocyte pathway (Table 1A).

### A.2 DC-Monocyte Tubes for Bone Marrow (BM) Analysis

In another embodiment, the above described DC-Monocyte backbone combination of 7 markers (CD14. CD16. CD33, CD141, CD300e. CD303, HILA-DR), combined with 6 or 7 labelings, can be supplemented with the four markers CD34, CD45, CD64 and CD117 (Table 1B), which would allow for bone marrow studies, in particular the identification and basic subsetting of monocytic precursors and mature cells (monoblasts CD34+/CD117+, monoblasts CD34−/CD117+, promonocytes CD14⁻, promonocytes CD14+, cMo, iMo and ncMo), myDC CD141+, pDC, mature basophils and mast cells (FIG. 3B):
Monoblasts CD34+/CD117+: HLA-DR⁺⁺, CD14⁻, CD16⁻, CD33⁺, CD34⁺, CD45⁺, CD64⁺, CD117⁺, CD141⁻, CD300e⁻, CD303⁻
Monoblasts CD344CD117+: HLA-DR⁺, CD14⁻, CD16⁻, CD33⁺, CD34⁻, CD45⁺, CD64⁺, CD117+. CD141⁻, CD300e⁻, CD303⁻
Promonocytes CD14^{-/low}: HLA-DR⁺⁺, CD14^{-/low}, CD16⁻, CD33⁺, CD34⁻, CD45⁺, CD64⁺, CD117⁻, CD141⁻, CD300e⁻, CD303⁻
Promonocytes CD14+: HLA-DR⁺⁺, CD14⁺, CD16⁻, CD33⁺, CD34⁻, CD45⁺, CD64⁺, CD117⁻, CD141⁻, CD300e⁻, CD303⁻
cMo: HLA-DR⁺⁺, CD14⁺, CD16⁻, CD33⁺, CD34⁻, CD45⁺, CD64⁺, CD117⁻, CD141⁻, CD300e⁺, CD303⁻
iMo: HLA-DR⁺⁺, CD14⁺, CD16⁺, CD33⁺, CD34⁻, CD45⁺, CD64⁺, CD117⁻, CD141⁻, CD300e⁺, CD303⁻
ncMo: HLA-DR⁺⁺, CD14⁺, CD16⁺, CD33⁺, CD34⁻, CD45⁺, CD64⁺, CD117⁻, CD141⁻, CD300e⁺, CD303⁻
myDC CD141+: HLA-DR⁺⁺, CD14⁻, CD16⁻, CD33⁺⁺, CD34⁻, CD45⁺, CD64⁻, CD117^{-/low}, CD141⁺⁺, CD300e⁻, CD303⁻
pDC: HLA-DR⁺⁺, CD14⁻, CD16⁻, CD33^{-/low}, CD34⁻, CD45¹, CD64⁻, CD117⁻, CD141⁺, CD300e⁻, CD303⁺
Mature basophils: HLA-DR⁻, CD14⁻, CD16⁻, CD33⁺, CD34⁻, CD45^{low}, CD64⁻, CD117⁻, CD141⁻, CD300e⁻, CD303⁻
Mast cells: HLA-DR⁻, CD14⁻, CD16⁻, CD33⁺, CD34⁻, CD45⁺, CD64^{-/+}, CD117⁺⁺, CD141⁻, CD300e⁻, CD303⁻

Addition of CD36 to the 11 marker combination, combined with 10 or 11 labelings, would further allow for the evaluation of pDC precursors (HLA-DR⁺⁺, CD14⁻, CD16⁻, CD33⁺, CD34^{-/+}, CD36⁺, CD45⁺, CD64⁻, CD117^{-/+}, CD141^{-/+}, CD300e⁻, CD303^{-/+}), myDC precursors (HLA-DR⁺⁺, CD14⁻, CD16⁻, CD33⁺⁺, CD34^{-/+}, CD36^{1ow}, CD45⁺, CD64^{-/+}, CD117^{-/+}, CD141⁺, CD300e⁻, CD303⁻), nucleated erythroid precursors (HLA-DR^{-/low}, CD11b⁻, CD13⁻, CD14−, CD16⁻, CD33⁻, CD34^{-/+}, CD35⁻, CD36⁺, CD45^{-/low}, CD64⁻, CD117^{-/+}, CD141⁻, CD163⁻, CD300e⁻, CD303⁻) and further subsetting of promonocytes into promonocytes CD14^{-/low} CD36⁻ and promonocytes CD14^{-/low} CD36⁺ (Table 1B; FIG. 3B).

Extension of the combination described above (12 markers combined with 11 or 12 labelings), by inclusion of CD11b, CD13 and CD35 (Table 1B, FIG. 3B) would allow further detailed subsetting of promonocytes, as well as the identification of mesenchymal stem cells, eosinophils and different maturation stages of the neutrophil-granulocyte lineage:
Promonocytes CD14−/CD11b−/CD36−/CD35−: HLA-DR⁺⁺, CD11b⁻, CD13⁺, CD14⁻, CD16⁻, CD33⁺, CD34⁻, CD35⁻, CD36⁻, CD45⁺, CD64⁺, CD117⁻, CD141⁻, CD300e⁻, CD303⁻
Promonocytes CD14−/CD11b+/CD36+/CD35−: HLA-DR⁺⁺, CD11b⁺, CD13⁺, CD14⁻, CD16⁻, CD33⁺, CD34⁻, CD35⁻, CD36⁺, CD45⁺, CD64⁺, CD117⁻, CD141⁻, CD300e⁻, CD303⁻
Promonocytes CD14low/CD11b+/CD36+/CD35+: HLA-DR⁺⁺, CD11b⁺, CD13⁺, CD14^{low}, CD16⁻, CD33⁺, CD34⁻, CD35⁺, CD36⁺, CD45⁺, CD64⁺, CD117⁻, CD141⁻, CD300e⁻, CD303⁻
Mesenchymal stem cells: HLA-DR^{-/+}, CD11b⁻, CD13⁺⁺⁺, CD14⁻, CD16⁻, CD33⁻, CD34⁻, CD35⁻, CD36⁻, CD45⁻, CD64⁻, CD117⁻, CD141⁻, CD300e⁻, CD303⁻
Eosinophils: HLA-DR⁻, CD11b⁺, CD13^{-/+}, CD14⁻, CD16⁻, CD33^{low}, CD34⁻, CD35^{-/+}, CD36⁻, CD45⁺⁺, CD64⁻, CD117⁻, CD141⁻, CD300e⁻, CD303⁻
Myeloblast: HLA-DR⁺, CD11b⁻, CD13⁺⁺, CD14⁻, CD16⁻, CD33⁺, CD34⁺, CD35⁻, CD36⁻, CD45⁺, CD64⁻, CD117⁺, CD141⁻, CD300e⁻, CD303⁻
Promyelocyte: HLA-DR⁻, CD11b⁻, CD13⁺⁺, CD14⁻, CD16⁻, CD33⁺, CD34⁻, CD35⁻, CD36⁻, CD45′, CD64′, CD117^{+/low}, CD141⁻, CD300e⁻, CD303⁻
Myelocyte: HLA-DR⁻, CD11b⁻, CD13⁻, CD14⁻, CD16⁻, CD33⁺, CD34⁻, CD35⁻, CD36⁻, CD45⁺, CD64⁺⁺, CD117⁻, CD141⁻, CD300e⁻, CD303⁻
Metamyelocyte: HLA-DR⁻, CD11b⁺, CD13⁻, CD14⁻, CD16^{low}, CD33⁺, CD34⁻, CD35^{-/low}, CD36⁻, CD45⁺, CD64⁺⁺, CD117⁻, CD141⁻, CD300e⁻, CD303⁻
Band/segmented neutrophils: HLA-DR⁻, CD11bf+, CD13⁺⁺, CD14⁻, CD16⁺⁺, CD33⁺, CD34⁻, CD35⁺, CD36⁻, CD45⁺, CD64^{low}, CD117⁻, CD141⁻, CD300e⁻, CD303⁻

In another embodiment, only CD163 is added to the combination of 11 markers in order to allow for further division of promonocytes CD14⁺ into promonocytes CD14⁺ CD163⁻ and promonocytes CD14⁺ CD163⁻.

The backbone combination (7 markers combined with 6 or 7 labelings), supplemented with CD34, CD45, CD64 and CD117 (11 markers, combined with 10 or 11 labelings) can be further extended by including FcεRI and CD163. This combination enables the subsetting of promonocytes CD14⁺, as described above, as well as identification and subsetting of mature myDC CD1c+ and of basophil precursors (FIG. 3B):
Promonocytes CD14+/CD163−: HLA-DR⁺⁺, CD14⁺, CD16⁻, CD33⁺, CD34⁻, CD45⁺, CD64⁺, CD117⁻, CD141⁻, CD163⁻, CD300e⁻, CD303⁻, FcERI−
Promonocytes CD14+/CD163+: HLA-DR-+, CD14⁺, CD16⁻, CD33⁺, CD34⁻, CD45⁺, CD64⁺, CD117⁻, CD141⁻, CD163⁺, CD300e⁻, CD303⁻, FcεRI−
myDC CD1c+ CD14low: HLA-DR⁺⁺, CD14^{low}, CD16⁻, CD33⁺⁺, CD34⁻, CD45⁺, CD64^{low/+}, CD117⁻, CD141⁻, CD163⁺, CD300e⁻, CD303⁻, FcεRI+
myDC CD1c+ CD14− CD163+: HLA-DR⁺⁺, CD14⁻, CD16⁻, CD33⁺⁺, CD34⁻, CD45⁺, CD64^{low/+}, CD117⁻, CD141⁻, CD163⁺, CD300e⁻, CD303⁻, FcεRI+
myDC CD1c+ CD14− CD163+: HLA-DR⁺⁺, CD14⁻, CD16⁻, CD33⁺⁺, CD34⁻, CD45⁺, CD64^{low/+}, CD117⁻, CD141⁻, CD163⁻, CD300e⁻, CD303⁻, FcεRI+
Basophil precursors: HLA-DR^{-/low}, CD14⁻, CD16⁻, CD33⁺, CD34⁺, CD45^{low}, CD64⁻, CD117⁺, CD141⁻, CD163⁻, CD300e⁻, CD303⁻, FcεRI^{low}

Overall, for studies aimed at evaluating myeloid precursor cells and maturation patterns, the DC-Monocyte backbone comprised of 7 markers (CD14, CD16, CD33, CD141, CD300e, CD303, HLA-DR), can be supplemented with 10 additional makers (CD11b, CD13, CD34, CD35, CD36, CD45, CD64, CD117, CD163 and FcεRI), in a combination in which CD14 and CD34 can be evaluated with the same label, similarly to the markers CD300e and CD303 (Table 1B). This panel of 17 antibodies conjugated to 15 to 17 distinct detectable labels allows for the identification of 28 cell populations present in bone marrow (FIG. 3B), including mature and immature pDC, myDC precursors and mature myDC CD141+ and myDC CD1c+ (including the myDC CD1c+CD14^{low}, myDC CD1c⁺ CD14− CD163+ and myDC CD1c⁺ CD14− CD163⁻ subsets), mature monocytes (cMo, iMo and ncMo), as well as their precursors (monoblasts CD34⁺/CD117⁺ and CD34⁻/CD117⁺, promonocytes CD14⁻/CD11b⁻/CD36⁻/CD35⁻, CD14⁻/CD11b⁺/CD36⁺/CD35⁻, CD14^{low}/CD11b⁺/CD36⁺/CD35⁺, −CD14⁺/CD163⁻ and CD14⁺/CD163⁺). Furthermore, five populations of mature and immature neutrophils (myeloblasts, promyelocytes, myelocytes, metamyelocytes and band/segmented neutrophils), mature basophils and their precursor cells, eosinophils, mast cells, nucleated erythroid precursor cells and mesenchymal stem cells can also be identified using the above mentioned combination.

### A.3 Diagnosis, (Sub) Classification and Monitoring of Monocytic and Dendritic Cell Acute Leukemias

The composition of the above DC-Monocyte tubes for blood and BM studies allows for the identification of mature (e.g. peripheral blood) and immature myeloid cell populations (e.g. bone marrow), as well as for the evaluation of the maturation pathways of these cells, particularly of the monocytic and dendritic cell lineages.

The information provided by these unique antibody combinations cannot only be used for immune monitoring of e.g. infection, response to vaccination or immunotherapy, but are also perfectly suited for diagnosis and (sub)classification of acute leukemias of monocyte and DC origin, since they dissect the corresponding normal hematopoietic pathways as well as the leukemic deviations from these normal pathways. Moreover, these tubes also allow for monitoring of small monocytic and DC leukemic cell populations during and after treatment to assess treatment effectiveness.

TABLE 1A
Exemplary Antibody combinations for DC-Monocyte tubes for analysis of blood

| | | | | | | | | F8 e.g. |
|---|---|---|---|---|---|---|---|---|
| | F1 e.g. | F2 e.g. | F3 e.g. | F4 e.g. | F5 e.g. | F6 e.g. | F7 e.g. | PerCP |
| PB tubes | BV421 | BV510 | BV605 | BV650 | BV711 | BV786 | FITC | Cy5.5 |
| BB1 | CD141 | | | | HLA-DR | CD16 | | |
| BB2A | CD141 | CD5 | | | HLA-DR | CD16 | | |
| BB2B | CD141 | | | | HLA-DR | CD16 | | |
| BB2C | CD141 | | | | HLA-DR | CD16 | | |
| BB3A | CD141 | | | | HLA-DR | CD16 | | CD36 |
| BB3B | CD141 | | | CD62L | HLA-DR | CD16 | | |
| BB4 | CD141 | | CD192 | CD62L | HLA-DR | CD16 | | CD36 |
| DC/monocyte | CD141 | CD5 | CD192 | CD62L | HLA-DR | CD16 | | CD36 |
| tube | | | | | | | | |
| DC/monocyte | CD141 | CD5 | CD192 | CD62L | HLA-DR | CD16 | CD1c | CD36 |
| tube | | | | | | | | |
| DC/monocyte | CD141 | CD5 | CD192 | CD62L | HLA-DR | CD16 | CD1c | CD36 |
| tube | | | | | | | | |
| DC/monocyte | CD141 | CD5 | CD192 | CD62L | HLA-DR | CD16 | Axl | CD36 |
| tube | | | | | | | | |
| DC/monocyte | CD141 | CD5 | CD192 | CD62L | HLA-DR | CD16 | CD1c | CD36 |
| tube | | | | | | | | |

| | | F10 e.g. | F11 e.g. | | | | |
|---|---|---|---|---|---|---|---|
| | F9 e.g. | PE | PE | F12 e.g. | F13 e.g. | F14 e.g. | F15 e.g. |
| PB tubes | PE | CF594 | Cy5 | PE Cy7 | APC | AF700 | APC H7 |
| BB1 | | | | CD33 | CD300e | | CD14 |
| | | | | | CD303 | | |
| BB2A | | | | CD33 | CD300e | | CD14 |
| | | | | | CD303 | | |
| BB2B | | CD34 | | CD33 | CD300e | | CD14 |
| | | | | | CD303 | | |
| BB2C | | | | CD33 | CD300e | CD45 | CD14 |
| | | | | | CD303 | | |
| BB3A | SLAN | | | CD33 | CD300e | | CD14 |
| | | | | | CD303 | | |
| BB3B | FcERI | | | CD33 | CD300e | | CD14 |
| | | | | | CD303 | | |
| BB4 | | | | CD33 | CD300e | CD45 | CD14 |
| | | | | | CD303 | | |
| DC/monocyte | SLAN | CD34 | | CD33 | CD300e | CD45 | CD14 |
| tube | FcERI | | | | CD303 | | |
| DC/monocyte | SLAN | CD34 | CD100 | CD33 | CD300e | CD45 | CD14 |
| tube | FcERI | | | | CD303 | | |
| DC/monocyte | SLAN | CD34 | Axl | CD33 | CD300e | CD45 | CD14 |
| tube | FcERI | | | | CD303 | | |
| DC/monocyte | SLAN | CD34 | CD100 | CD33 | CD300e | CD45 | CD14 |
| tube | FcERI | | | | CD303 | | |
| DC/monocyte | SLAN | Axl | CD100 | CD33 | CD300e | CD45 | CD14 |
| tube | FcERI | | | | CD303 | | CD34 |

TABLE 1B
Antibody combinations for DC-Monocyte tubes for analysis of bone marrow

| | | | | | | | | F8 e.g. |
|---|---|---|---|---|---|---|---|---|
| | F1 e.g. | F2 e.g. | F3 e.g. | F4 e.g. | F5 e.g. | F6 e.g. | F7 e.g. | PerCP |
| BM Tubes | BV421 | BV510 | BV605 | BV650 | BV711 | BV786 | FITC | Cy5.5 |
| BM | CD141 | | | | HLA-DR | CD16 | CD117 | |
| mono/DC | | | | | | | | |
| precursors | | | | | | | | |
| BM | CD141 | | | | HLA-DR | CD16 | CD117 | CD36 |
| mono/DC | | | | | | | | |
| precursors | | | | | | | | |
| BM | CD141 | CD11b | | CD35 | HLA-DR | CD16 | CD117 | CD36 |
| mono/DC | | | | | | | | |
| precursors | | | | | | | | |
| BM | CD141 | | CD163 | | HLA-DR | CD16 | CD117 | |
| mono/BC | | | | | | | | |
| precursors | | | | | | | | |
| BM | CD141 | | CD163 | | HLA-DR | CD16 | CD117 | |
| mono/DC | | | | | | | | |
| precursors | | | | | | | | |
| BM | CD141 | CD11b | CD163 | CD35 | HLA-DR | CD16 | CD117 | CD36 |
| mono/DC | | | | | | | | |
| precursors | | | | | | | | |

| | | F10 e.g. | F11 e.g. | | | | |
|---|---|---|---|---|---|---|---|
| | F9 e.g. | PE | PE | F12 e.g. | F13 e.g. | F14 e.g. | F15 e.g. |
| BM Tubes | PE | CF594 | Cy5 | PE Cy7 | APC | AF700 | APC H7 |
| BM | CD64 | CD34 | | CD33 | CD300e | CD45 | CD14 |
| mono/DC | | | | | CD303 | | |
| precursors | | | | | | | |
| BM | CD64 | CD34 | | CD33 | CD300e | CD45 | CD14 |
| mono/DC | | | | | CD303 | | |
| precursors | | | | | | | |
| BM | CD64 | CD34 | CD13 | CD33 | CD300e | CD45 | CD14 |
| mono/DC | | | | | CD303 | | |
| precursors | | | | | | | |
| BM | CD64 | CD34 | | CD33 | CD300e | CD45 | CD14 |
| mono/BC | | | | | CD303 | | |
| precursors | | | | | | | |
| BM | CD64 | FcεRI | | CD33 | CD300e | CD45 | CD14 |
| mono/DC | | | | | CD303 | | CD34 |
| precursors | | | | | | | |
| BM | CD64 | FcεRI | CD13 | CD33 | CD300e | CD45 | CD14 |
| mono/DC | | | | | CD303 | | CD34 |
| precursors | | | | | | | |

### B.1 CD4 T-Cell Tubes

Previous studies have proposed combinations of markers to recognize follicular helper T (Tfh) cells, including some subsets of these cells. In parallel, other investigators taught how to identify Tregs in the absence of intracellular stainings and the possibility to define Th1, Th2, Th17, Th22, Th1/17, helper T-cell subsets based on both intracellular cytokine expression and surface membrane markers (Streitz et al., 2013; Mahnke et al., 2013a; Wingender et al., 2015; Finak et al., 2016). However, nobody has been able to simultaneously identify the above helper T-cell subsets within Tregs, Tfh and other CD4+ T-helper cells in association with their maturation stages, in a single antibody panel tube, particularly in the absence of intracellular staining procedures that might affect antigen expression profiles obtained for membrane markers that are critical for optimal identification of specific CD4+ T-cell subsets, e.g. Th1 and Th2 cells. Therefore, according to the present invention, there is no need for staining of intracellular markers for CD4+ T-cell subsetting.

Blood Tfh cells have been defined by co-expression of CD185 (CXCR5) and CD4. In addition, further characterization of these cells has been based on a combination of CD10, CD84, CD272, CD278 and/or CD279, particularly for Tfh-cells in tissues different from blood (Tangye et al., 2013).

Early definition of blood Tregs in the literature was based on co-expression of CD25^{high}, CD4 and intracellular FoxP3. Subsequent studies defined co-expression of CD25^{high} in the absence or presence of low levels of CD127 to be a surrogate phenotype for CD4-positive blood Tregs (Miyara et al., 2009). Further subsetting of Tregs based on CD15s and CD39 has been proposed previously.
In parallel, surrogate cell surface phenotypes have been also proposed for identification of:
1. Interferon gamma producing Th1 cells;
2. IL4 and IL5 producing Th2 cells;
3. IL17 producing Th17 cells;
4. IL22 producing Th22 cells,
5. Interferon gamma plus IL17-producing Th1/Th17 cells.
   These cell surrogate phenotypes are based on a combination of the four markers CD183 (CXCR3), CD194 (CCR4), CD196 (CCR6) and CCR10:
1. CD183+CD194-CD196-CCR10− for Th1;
2. CD183-CD194+CD196-CCR10− for Th2;
3. CD183-CD194+CD196+CCR10− for Th17;
4. CD183+CD194-CD196+CCR10− for Th1/fh17;
5. CD183-CD194+CD196+CCR10+ for Th22.
   Only Wingender et al. (2015) has taught how to distinguish within the above cell populations between conventional CD4 positive Th1, Th2, Th17, Th22, Th1/Th17 T-cells and CD4 positive Th1-like, Th2-like, Th17-like, Th22-like, Th1/Th17-like Tregs and Tfh T-cells. However, he failed to accurately classify these functional CD4 T-cell populations into maturation stages, particularly transitional memory and effector memory cells.

The present inventors succeeded in overcoming this problem by inclusion of the markers CD27 and CD62L in combination with CD45RA or CD45RO, which appeared to be more appropriate than the earlier proposed CD45RA and CD197 marker combination (Mahnke et al., 2013a; Wingender et al., 2015).

Accordingly, the invention provides in one embodiment a reagent composition for the cytometric immunophenotyping of leukocytes comprising antibodies conjugated to a detectable label, the conjugated antibodies being directed against the following combination of markers: CD27, CD45RA or CD45RO, CD62L, CD127, CD3, CD25, CCR10, CD183 (CXCR3), CD196 (CCR6), CD194 (CCR4), CD185 (CXCR5) and CD4. This reagent composition allows either identification of all previously defined subsets of Tregs, Tfh and other CD4+ T-helper cells. In addition, new subsets of these population are identified with the antibody combination proposed.

TABLE 2
Exemplary Antibody combinations for CD4 T-cell tube *, **, ***, ****, *****

| | | | | | | | | F8 e.g. | | F10 e.g. | | | | |
|---|---|---|---|---|---|---|---|---|---|---|---|---|---|---|
| | F1 e.g. | F2 e.g. | F3 e.g. | F4 e.g. | F5 e.g. | F6 e.g. | F7 e.g, | PerCP | F9 e.g. | PE | F11 e.g. | F12 e.g. | F13 e.g. | F14 e.g. |
| | BV421 | BV510 | BV605 | BV650 | BV711 | BV786 | FITC | Cy5.5 | PE | CF594 | PE Cy7 | APC | AF700 | APC H7 |
| 1 | CD27 | CD45RA | | CD62L | CD127 | CD3 | CD25 | CCR10 | CD183 | CD196 | CD194 | CD185 | | CD4 |
| | | | | | | | | | (CXCR3) | (CCR6) | (CCR4) | (CXCR5) | | |
| 1A | CD27 | CD45RA | | CD62L | CD127 | CD3 | CD25 | CCR10 | CD183 | CD196 | CD194 | CD185 | CD45 | CD4 |
| | | | | | | | | | (CXCR3) | (CCR6) | (CCR4) | (CXCR5) | | |
| 1B | CD27 | CD45RA | CD8 | CD62L | CD127 | CD3 | CD25 | CCR10 | CD183 | CD196 | CD194 | CD185 | | CD4 |
| | | | | | | | | | (CXCR3) | (CCR6) | (CCR4) | (CXCR5) | | |
| 1C | CD27 | CD45RA | CD8 | CD62L | CD127 | CD3 | CD25 | CCR10 | CD183 | CD196 | CD194 | CD185 | CD45 | CD4 |
| | | | | | | | | | (CXCR3) | (CCR6) | (CCR4) | (CXCR5) | | |
| 1D | CD27 | CD45RA | CD8 | CD62L | CD16 | CD3 | CD57 | CD28 | cyGranB | TcRγδ | CD335 | CD56 | CD45 | CD4 |
| | | | | | | | | | | | (NKp46) | | | |
| 2 | CD27 | CD45RO | | CD62L | CD127 | CD3 | CD25 | CCR10 | CD183 | CD196 | CD194 | CD185 | | CD4 |
| | | | | | | | | | (CXCR3) | (CCR6) | (CCR4) | (CXCR5) | | |
| 2A | CD27 | CD45RO | | CD62L | CD127 | CD3 | CD25 | CCR10 | CD183 | CD196 | CD194 | CD185 | CD45 | CD4 |
| | | | | | | | | | (CXCR3) | (CCR6) | (CCR4) | (CXCR5) | | |
| 2B | CD27 | CD45RO | CD8 | CD62L | CD127 | CD3 | CD25 | CCR10 | CD183 | CD196 | CD194 | CD185 | | CD4 |
| | | | | | | | | | (CXCR3) | (CCR6) | (CCR4) | (CXCR5) | | |
| 2C | CD27 | CD45RO | CD8 | CD62L | CD127 | CD3 | CD25 | CCR10 | CD183 | CD196 | CD194 | CD185 | CD45 | CD4 |
| | | | | | | | | | (CXCR3) | (CCR6) | (CCR4) | (CXCR5) | | |
| 2D | CD27 | CB45RO | CD8 | CD62L | CD16 | CD3 | CD57 | CD28 | cyGranB | TcRγδ | CD335 | CD56 | CD45 | CD4 |
| | | | | | | | | | | | (NKp46) | | | |

* The proposed tubes can be further extended with CD31 for detection of recent thymic emigrants (RTE) and/or with CD95 for detection of CD95hi stem cell memory CD4+ T-cells.
** The proposed tubes can be further extended with activation markers CD278 (ICOS), CD279 (PD1) and/or HLA-DR, which can contribute to better definition of the activated CD4+ T-cell compartment.
*** The proposed tubes can be further extended with antibodies against TcR-Vβ and/or TcR-Vα domains (Table 6) plus an anti-TcRγδ antibody; or with antibodies against TcR-Vδ and/or TcR-Vγ domains plus an anti-TcRαβ antibody.
**** The proposed tubes 1B. 1C, 2B and 2C can be further extended with antibodies against cytotoxic cell populations other than CD8+ T cells (i.e. CD16, CD56, and/or TCRγδ) together with cytotoxic-related markers (such as CD28, CD57, and/or granzyme B).
***** The proposed tubes can be further extended with antibodies against mutually exclusive TcR-Cβ1 and TcR-Cβ2 epitopes, such as the epitope recognized by the JOVI-1 antibody (BD Biosciences) for detection of diversity in the TcRαβ+ T-cell compartment, particularly in TcRαβ+ T-cells that are not recognized by the antibodies against TcR-Vβ and/or TcR-Vα domains (Table 6)

Provided herein is a unique combination of conjugated antibodies against 12 cell surface markers (CD3, CD4, CD25, CD27, CD62L, CD127, CD183, CD185, CD194, CD196, CCR10, and CD45RA or CD45RO) to which conjugated antibodies against CD8 and/or CD45 may be added (Table 2). This reagent identifies an unprecedentedly high number of at least 89 well-defined subsets of CD4 positive T-cells in 200 μL of normal blood (Table 3), including all previously defined functional subsets of conventional Th1, Th2. Th17, Th22, Th1/Th17 T-cells and CD4 positive Th1-like, Th2-like, Th17-like, Th22-like, Th1/Th17-like Tregs and Tfh T-cells, subdivided in 5 maturation stages of naïve, central memory, transitional memory, effector memory and terminal effector CD4 T-cells.

FIG. 4 illustrates the strategy for recognition of the CD4+ T-cell subsets. The specific immunophenotypic criteria for the definition of each of the 89 CD4 positive T-cell subsets, as well as their functional correlates and their relative frequency within the blood CD4 positive T-cell compartment are summarized in Table 3. In addition, the “population tree” in FIG. 5 shows how these CD4+ T-cell subsets are linked. Of note, the above 12-marker combination will reproducibly identify at least 89 subsets in 100-200 μL of blood. If the amount of blood analyzed is further increased, a higher number of 161 CD4 positive T-cell subsets are identified (frequency <0.1 cell per μL) (Table 4).

The above 12-marker combination (with or without CD45 and/or CD8) can be further extended with CD31 for more accurate detection of recent thymic emigrants (RTE) (Kohler et al., 2009) and/or with CD95 for more accurate detection of CD95^{high} stem cell memory CD4+ T-cells (Gattinoni et al., 2011). Accordingly, the reagent composition may further comprise conjugated antibodies against CD31 and/or CD95.

Furthermore, the above marker combinations can be further extended with the CD69, CD278 (ICOS), CD279 (PD1) and/or HLA-DR activation markers (Mahnke et al., 2013b; McAdam et al., 2001), which can contribute to a better definition of the activated CD4+ T-cell compartment in infectious diseases, vaccination studies, and other settings with an activated CD4+ T-cell compartment.

For the simultaneous study of cytotoxic cell populations and their different maturation-associated cell subsets, a panel of antibodies aiming at the identification of additional cytotoxic subsets is preferably composed with antibodies identifying TCRγδ (anti-TCRγδ) and NK cells (i.e. CD16, CD56, and/or CD335 antibodies) together with cytotoxic-related markers (i.e. antibodies against CD28, CD57 and/or granzymeB).

The above antibody panels for CD4 and cytotoxic T-cells can be supplemented with reagents that allow for antigen-specific stainings. For example, a CD4 T-cell reagent composition comprises one or more antigen-specific peptide(s) or peptide pools, which peptides may be derived from micro-organisms, allergens, auto-antigens, vaccines, or immunotherapeutic components. Such peptides or peptide pools for staining the antigen-specific T-cells are preferably presented via MHC molecules, such as via tetramer systems or other systems known in the art, or they may be bound to labels that carry one or more reporter compounds. A reagent composition according to the invention may comprise one or more antigen-specific peptides in MHC molecules for detection and enumeration of antigen-specific T-cells, which MHC molecules are included in multimeric constructs that may be directly or indirectly conjugated to a detectable label. In one embodiment, the composition comprises at least one peptide that is presented in the appropriate MHC molecules via the Klickmer or Dextramer system (Immudex, Copenhagen, Denmark).

In one embodiment, any of the reagent compositions comprising antibodies against the set of markers shown in Table 2 can be further extended with antibodies against mutually exclusive TcR-Cβ1 and TcR-Cβ2 epitopes, such as the epitope recognized by the JOVI-1 antibody (BD Biosciences) for detection of diversity in the TcRαβ+ T-cell compartment, particularly in TcRαβ+ T-cells that are not recognized by the antibodies against TcR-Vβ and/or TcR-Vα domains (see Table 6). Accordingly, also provided herein is a reagent, composition for detection of diversity vs. clonality in TcRαβ+ T-cells, comprising conjugated antibodies against CD3, CD4, CD25, CD27, CD62L, CD127, CD183, CD185, CD194, CD196, CCR10, and CD45RA or CD45RO, to which conjugated antibodies against CD8 and/or CD45 may be added, and further comprising conjugated antibodies against mutually exclusive TcR-Cβ1 and/or TcR-Cβ2 epitopes.

In a specific embodiment, the results of the CD4 T-cell tube (CD3, CD4, CD25, CD27, CD62L, CD127, CD183, CD185, CD194, CD196, CCR10, and CD45RA or CD45RO; see Table 2, tube 1C or 2C), preferably supplemented with CD8 and CD45 can typically be merged and calculated with the results of the corresponding “sister tube” for Cytotoxic T & NK cells, using the 7 markers CD3, CD4, CD8, CD27, CD45, CD62L, and CD45RA or CD45RO as backbone marker set (bold in Table 2) according to the EuroFlow guidelines (Kalina et al., 2012) (www.EuroFlow.org). This Cytotoxic T & NK cell tube further comprises CD8, CD16, CD28, CD56, CD57, CD335, TcRγδ, and/or granzyme B (see tube 1D or 2D of Table 2).

Accordingly, the invention also provides a set of reagent compositions, comprising (i) any of the CD4 T cell reagent compositions as mentioned herein above; and (i) a reagent composition comprising conjugated antibodies against CD3, CD4, CD8, CD27, CD45, CD62L, CD45RA or CD45RO, CD16, CD28, CD56, CD57, TCRγδ, optionally supplement with conjugated antibodies against the markers CD335 and/or granzyme B. Exemplary reagents sets comprise tubes 1C and 1D, or tubes 2C and 2D, as shown in Table 2.

TABLE 3
89 CD4 T-cell subsets

| Peripheral blood CD4+ T-cell populations | | | | | | | |
|---|---|---|---|---|---|---|---|
| (CD3+CD4+CD45++) | CD25 | CD27 | CD45RA | CD62L | CD127 | CD154* | CD183 |
| CD4+ naïve T cells | − | + | + | + | + | − | − |
| CD4+ Th1 cells | −/+ | −/+ | −/+ | −/+ | −/+ | − | + |
| Central memory Th1 | lo/+ | + | − | + | + | − | + |
| Transitional memory Th1 | lo/+ | + | − | − | + | − | + |
| Effector memory Th1 | − | − | − | −/+ | −/+ | − | + |
| Terminal effector Th1 | − | − | + | −/+ | −/+ | − | + |
| CD4+ Th2 cells | lo/+ | −/+ | −/+ | −/+ | −/+ | − | − |
| Central memory Th2 | lo/+ | + | − | + | + | − | − |
| Transitional memory Th2 | lo/+ | + | − | − | + | − | − |
| Effector memory Th2 | lo/+ | − | − | −/+ | −/+ | − | − |
| Terminal effector Th2 | lo/+ | − | + | −/+ | −/+ | − | − |
| CD4+ Th17 cells | lo/+ | −/+ | −/+ | −/+ | −/+ | − | − |
| Central memory Th17 | lo/+ | + | − | + | + | − | − |
| Transitional memory Th17 | lo/+ | + | − | − | + | − | − |
| Effector memory Th17 | lo/+ | − | − | −/+ | −/+ | − | − |
| Terminal effector Th17 | lo/+ | − | + | −/+ | −/+ | − | − |
| CD4+ Th1/Th17 cells | lo/+ | −/+ | −/+ | −/+ | −/+ | − | + |
| Central memory Th1/Th17 | lo/+ | + | − | + | + | − | + |
| Transitional memory Th1/Th17 | lo/+ | + | − | − | + | − | + |
| Effector memory Th1/Th17 | lo/+ | − | − | −/+ | −/+ | − | + |
| Terminal effector Th1/Th17 | lo/+ | − | + | −/+ | −/+ | − | + |
| CD4+ Th22 cells | lo/+ | −/+ | −/+ | −/+ | −/+ | − | − |
| Central memory Th22 | lo/+ | + | − | + | + | − | − |
| Transitional memory Th22 | lo/+ | + | − | − | + | − | − |
| Effector memory Th22 | lo/+ | − | − | −/+ | −/+ | − | − |
| Terminal effector Th22 | lo/+ | − | + | −/+ | −/+ | − | − |
| CD4+ CD183+ CD194+ CD196+ CCR10+ | lo/+ | −/+ | −/+ | −/+ | −/+ | − | + |
| CM CD4+ CD183+ CD194+ CD196+ CCR10+ | lo/+ | + | − | + | + | − | + |
| TM CD4+ CD183+ CD194+ CD196+ CCR10+ | lo/+ | + | − | − | + | − | + |
| EM CD4+ CD183+ CD194+ CD196+ CCR10+ | lo/+ | − | − | −/+ | −/+ | − | + |
| TE CD4+ CD183+ CD194+ CD196+ CCR10+ | lo/+ | − | + | −/+ | −/+ | − | + |
| CD4+ CD183+ CD194+ CD196+ CCR10− | lo/+ | −/+ | −/+ | −/+ | −/+ | − | + |
| CM CD4+ CD183+ CD194+ CD196+ CCR10− | lo/+ | + | − | + | + | − | + |
| TM CD4+ CD183+ CD194+ CD196+ CCR10− | lo/+ | + | − | − | + | − | + |
| EM CD4+ CD183+ CD194+ CD196+ CCR10− | lo/+ | − | − | −/+ | −/+ | − | + |
| TE CD4+ CD183+ CD194+ CD196+ CCR10− | lo/+ | − | + | −/+ | −/+ | − | + |
| CD4+ CD183+ CD194+ CD196− CCR10+ | lo/+ | −/+ | −/+ | −/+ | −/+ | − | + |
| CM CD4+ CD183+ CD194+ CD196− CCR10+ | lo/+ | + | − | + | + | − | + |
| TM CD4+ CD183+ CD194+ CD196− CCR10+ | lo/+ | + | − | − | + | − | + |
| EM CD4+ CD183+ CD194+ CD196− CCR10+ | lo/+ | − | − | −/+ | −/+ | − | + |
| TE CD4+ CD183+ CD194+ CD195− CCR10+ | lo/+ | − | + | −/+ | −/+ | − | + |
| CD4+ CD183+ CD194+ CD196− CCR10− | lo/+ | −/+ | −/+ | −/+ | −/+ | − | + |
| CM CD4+ CD183+ CD194+ CD196− CCR10− | lo/+ | + | − | + | + | − | + |
| TM CD4+ CD183+ CD194+ CD196− CCR10− | lo/+ | + | − | − | + | − | + |
| EM CD4+ CD183+ CD194+ CD196− CCR10− | lo/+ | − | − | −/+ | −/+ | − | + |
| TE CD4+ CD183+ CD194+ CD196− CCR10− | lo/+ | − | + | −/+ | −/+ | − | + |
| CD4+ CD183+ CD194− CD196+ CCR10+ | lo/+ | −/+ | −/+ | −/+ | −/+ | − | + |
| CM CD4+ CD183+ CD194− CD196+ CCR10+ | lo/+ | + | − | + | + | − | + |
| TM CD4+ CD183+ CD194− CD196+ CCR10+ | lo/+ | + | − | − | + | − | + |
| EM CD4+ CD183+ CD194− CD196+ CCR10+ | lo/+ | − | − | −/+ | −/+ | − | + |
| TE CD4+ CD183+ CD194− CD196+ CCR10+ | lo/+ | − | + | −/+ | −/+ | − | + |
| CD4+ CD183+ CD194− CD196− CCR10+ | lo/+ | −/+ | −/+ | −/+ | −/+ | − | + |
| CM CD4+ CD183+ CD194− CD196− CCR10+ | lo/+ | + | − | + | + | − | + |
| TM CD4+ CD183+ CD194− CD196− CCR10+ | lo/+ | + | − | − | + | − | + |
| EM CD4+ CD183+ CD194− CD196− CCR10+ | lo/+ | − | − | −/+ | −/+ | − | + |
| TE CD4+ CD183+ CD194− CD196− CCR10+ | lo/+ | − | + | −/+ | −/+ | − | + |
| CD4+ CD183− CD194− CD196+ CCR10− | lo/+ | −/+ | −/+ | −/+ | −/+ | − | − |
| CM CD4+ CD183− CD194− CD196+ CCR10− | lo/+ | + | − | + | + | − | − |
| TM CD4+ CD183− CD194− CD196+ CCR10− | lo/+ | + | − | − | + | − | − |
| EM CD4+ CD183− CD194− CD196+ CCR10− | lo/+ | − | − | −/+ | −/+ | − | − |
| TE CD4+ CD183− CD194− CD196+ CCR10− | lo/+ | − | + | −/+ | −/+ | − | − |
| CD4+ CD183− CD194+ CD196− CCR10+ | lo/+ | −/+ | −/+ | −/+ | −/+ | − | − |
| CM CD4+ CD183− CD194+ CD196− CCR10+ | lo/+ | + | − | + | + | − | − |
| TM CD4+ CD183− CD194+ CD196− CCR10+ | lo/+ | + | − | − | + | − | − |
| EM CD4+ CD183− CD194+ CD196− CCR10+ | lo/+ | − | − | −/+ | −/+ | − | − |
| TE CD4+ CD183− CD194+ CD196− CCR10+ | lo/+ | − | + | −/+ | −/+ | − | − |
| non-naïve CD4+ CD183− CD194− CD196− CCR10− | lo/+ | −/+ | − | −/+ | | − | − |
| CD4+ Tregs | +hi | −/+ | −/+ | −/+ | +lo | − | −/+ |
| Naïve Treg | +hi | + | + | + | +lo | − | − |
| Th1-like Treg | +hi | −/+ | −/+ | −/+ | +lo | − | + |
| Th2-like Treg | +hi | −/+ | −/+ | −/+ | +lo | − | − |
| Th17-like Treg | +hi | −/+ | −/+ | −/+ | +lo | − | − |
| Th22-like Treg | +hi | −/+ | −/+ | −/+ | +lo | − | − |
| CD183+ CD194+ CD196− CCR10+ Treg | +hi | −/+ | −/+ | −/+ | +lo | − | + |
| CD183+ CD194+ CD196− CCR10− Treg | +hi | −/+ | −/+ | −/+ | +lo | − | + |
| CD183+ CD194+ CD196+ CCR10− Treg | +hi | −/+ | −/+ | −/+ | +lo | − | + |
| CD183+ CD194+ CD196+ CCR10+ Treg | +hi | −/+ | −/+ | −/+ | +lo | − | + |
| CD183− CD194+ CD196− CCR10+ Treg | +hi | −/+ | −/+ | −/+ | +lo | − | − |
| CD4+ Tfh cells | −/lo | −/+ | −/+ | −/+ | −/+ | − | −/+ |
| Naïve Tfh | −/lo | + | + | + | + | − | − |
| Treg Tfh | −/lo | −/+ | −/+ | −/+ | +lo | − | −/+ |
| Th1-like Tfh | −/lo | −/+ | −/+ | −/+ | + | − | + |
| Th2-like Tfh | −/lo | −/+ | −/+ | −/+ | + | − | − |
| Th17-like Tfh | −/lo | −/+ | −/+ | −/+ | + | − | − |
| Th1/Th17-like Tfh | −/lo | −/+ | −/+ | −/+ | + | − | + |
| CD183+ CD194+ CD196− CCR10− Tfh | −/lo | −/+ | −/+ | −/+ | + | − | + |
| CD183+ CD194+ CD196+ CCR10− Tfh | −/lo | −/+ | −/+ | −/+ | + | − | + |
| CD183− CD194− CD196+ CCR10− Tfh | −/lo | −/+ | −/+ | −/+ | + | − | − |
| CD183− CD194− CD196− CCR10− Tfh | −/lo | −/+ | −/+ | −/+ | + | − | − |

| | | | | | Frequency |
|---|---|---|---|---|---|
| Peripheral blood CD4+ T-cell populations | | | | | (from total |
| (CD3+CD4+CD45++) | CD185 | CD194 | CD196 | CCR10 | CD4+ T cells) |
| CD4+ naïve T cells | − | − | − | − | >10% |
| CD4+ Th1 cells | − | − | − | − | >10% |
| Central memory Th1 | − | − | − | − | 5-10% |
| Transitional memory Th1 | − | − | − | − | 1-5% |
| Effector memory Th1 | − | − | − | − | 1-5% |
| Terminal effector Th1 | − | − | − | − | <0.5% |
| CD4+ Th2 cells | − | + | − | − | 1-5% |
| Central memory Th2 | − | + | − | − | 1-5% |
| Transitional memory Th2 | − | + | − | − | <0.5% |
| Effector memory Th2 | − | + | − | − | <0.5% |
| Terminal effector Th2 | − | + | − | − | <0.5% |
| CD4+ Th17 cells | − | + | + | − | 1-5% |
| Central memory Th17 | − | + | + | − | 1-5% |
| Transitional memory Th17 | − | + | + | − | 0.5-1% |
| Effector memory Th17 | − | + | + | − | <0.5% |
| Terminal effector Th17 | − | + | + | − | <0.5% |
| CD4+ Th1/Th17 cells | − | − | + | − | 1-5% |
| Central memory Th1/Th17 | − | − | + | − | 1-5% |
| Transitional memory Th1/Th17 | − | − | + | − | 1-5% |
| Effector memory Th1/Th17 | − | − | + | − | 0.5-1% |
| Terminal effector Th1/Th17 | − | − | + | − | <0.5% |
| CD4+ Th22 cells | − | + | + | + | 1-5% |
| Central memory Th22 | − | + | + | + | <0.5% |
| Transitional memory Th22 | − | + | + | + | 0.5-1% |
| Effector memory Th22 | − | + | + | + | 0.5-1% |
| Terminal effector Th22 | − | + | + | + | <0.5% |
| CD4+ CD183+ CD194+ CD196+ CCR10+ | − | + | + | + | 1-5% |
| CM CD4+ CD183+ CD194+ CD196+ CCR10+ | − | + | + | + | <0.5% |
| TM CD4+ CD183+ CD194+ CD196+ CCR10+ | − | + | + | + | 0.5-1% |
| EM CD4+ CD183+ CD194+ CD196+ CCR10+ | − | + | + | + | <0.5% |
| TE CD4+ CD183+ CD194+ CD196+ CCR10+ | − | + | + | + | <0.5% |
| CD4+ CD183+ CD194+ CD196+ CCR10− | − | + | + | − | 5-10% |
| CM CD4+ CD183+ CD194+ CD196+ CCR10− | − | + | + | − | 1-5% |
| TM CD4+ CD183+ CD194+ CD196+ CCR10− | − | + | + | − | 1-5% |
| EM CD4+ CD183+ CD194+ CD196+ CCR10− | − | + | + | − | 0.5-1% |
| TE CD4+ CD183+ CD194+ CD196+ CCR10− | − | + | + | − | <0.5% |
| CD4+ CD183+ CD194+ CD196− CCR10+ | − | + | − | + | 0.5-1% |
| CM CD4+ CD183+ CD194+ CD196− CCR10+ | − | + | − | + | <0.5% |
| TM CD4+ CD183+ CD194+ CD196− CCR10+ | − | + | − | + | <0.5% |
| EM CD4+ CD183+ CD194+ CD196− CCR10+ | − | + | − | + | <0.5% |
| TE CD4+ CD183+ CD194+ CD195− CCR10+ | − | + | − | + | <0.5% |
| CD4+ CD183+ CD194+ CD196− CCR10− | − | + | − | − | 5-10% |
| CM CD4+ CD183+ CD194+ CD196− CCR10− | − | + | − | − | 1-5% |
| TM CD4+ CD183+ CD194+ CD196− CCR10− | − | + | − | − | 1-5% |
| EM CD4+ CD183+ CD194+ CD196− CCR10− | − | + | − | − | 0.5-1% |
| TE CD4+ CD183+ CD194+ CD196− CCR10− | − | + | − | − | <0.5% |
| CD4+ CD183+ CD194− CD196+ CCR10+ | − | − | + | + | <0.5% |
| CM CD4+ CD183+ CD194− CD196+ CCR10+ | − | − | + | + | <0.5% |
| TM CD4+ CD183+ CD194− CD196+ CCR10+ | − | − | + | + | <0.5% |
| EM CD4+ CD183+ CD194− CD196+ CCR10+ | − | − | + | + | <0.5% |
| TE CD4+ CD183+ CD194− CD196+ CCR10+ | − | − | + | + | <0.5% |
| CD4+ CD183+ CD194− CD196− CCR10+ | − | − | − | + | <0.5% |
| CM CD4+ CD183+ CD194− CD196− CCR10+ | − | − | − | + | <0.5% |
| TM CD4+ CD183+ CD194− CD196− CCR10+ | − | − | − | + | <0.5% |
| EM CD4+ CD183+ CD194− CD196− CCR10+ | − | − | − | + | <0.5% |
| TE CD4+ CD183+ CD194− CD196− CCR10+ | − | − | − | + | <0.5% |
| CD4+ CD183− CD194− CD196+ CCR10− | − | − | + | − | 0.5-1% |
| CM CD4+ CD183− CD194− CD196+ CCR10− | − | − | + | − | 0.5-1% |
| TM CD4+ CD183− CD194− CD196+ CCR10− | − | − | + | − | <0.5% |
| EM CD4+ CD183− CD194− CD196+ CCR10− | − | − | + | − | <0.5% |
| TE CD4+ CD183− CD194− CD196+ CCR10− | − | − | + | − | <0.5% |
| CD4+ CD183− CD194+ CD196− CCR10+ | − | + | − | + | 0.5-1% |
| CM CD4+ CD183− CD194+ CD196− CCR10+ | − | + | − | + | <0.5% |
| TM CD4+ CD183− CD194+ CD196− CCR10+ | − | + | − | + | <0.5% |
| EM CD4+ CD183− CD194+ CD196− CCR10+ | − | + | − | + | <0.5% |
| TE CD4+ CD183− CD194+ CD196− CCR10+ | − | + | − | + | <0.5% |
| non-naïve CD4+ CD183− CD194− CD196− CCR10− | − | − | − | − | 0.5-1% |
| CD4+ Tregs | −/+ | −/+ | −/+ | −/+ | 5-10% |
| Naïve Treg | − | − | − | − | <0.5% |
| Th1-like Treg | − | − | − | − | <0.5% |
| Th2-like Treg | − | + | − | − | 0.5-1% |
| Th17-like Treg | − | + | + | − | 0.5-1% |
| Th22-like Treg | − | + | + | + | 1-5% |
| CD183+ CD194+ CD196− CCR10+ Treg | − | + | − | + | <0.5% |
| CD183+ CD194+ CD196− CCR10− Treg | − | + | − | − | <0.5% |
| CD183+ CD194+ CD196+ CCR10− Treg | − | + | + | − | 0.5-1% |
| CD183+ CD194+ CD196+ CCR10+ Treg | − | + | + | + | 0.5-1% |
| CD183− CD194+ CD196− CCR10+ Treg | − | + | − | + | <0.5% |
| CD4+ Tfh cells | + | −/+ | −/+ | − | >10% |
| Naïve Tfh | + | − | − | − | 0.5-1% |
| Treg Tfh | + | −/+ | −/+ | − | 1-5% |
| Th1-like Tfh | + | − | − | − | 1-5% |
| Th2-like Tfh | + | + | − | − | 1-5% |
| Th17-like Tfh | + | + | + | − | 5-10% |
| Th1/Th17-like Tfh | + | − | + | − | 1-5% |
| CD183+ CD194+ CD196− CCR10− Tfh | + | + | − | − | 1-5% |
| CD183+ CD194+ CD196+ CCR10− Tfh | + | + | + | − | 1-5% |
| CD183− CD194− CD196+ CCR10− Tfh | + | − | + | − | 1-5% |
| CD183− CD194− CD196− CCR10− Tfh | + | − | − | − | 1-5% |

*CD154 is only expressed by activated T cells
lo: low;
hi: high

TABLE 4
161 CD4 T-cell subsets

| Peripheral blood CD4+ T-Cell populations | | | | | | | |
|---|---|---|---|---|---|---|---|
| (CD3+CD4+CD45++) | CD25 | CD27 | CD45RA | CD62L | CD127 | CD154* | CD183 |
| CD4+ naïve T cells | − | + | + | + | + | − | − |
| CD4+ Th1 cells | −/+ | −/+ | −/+ | −/+ | −/+ | − | + |
| Central memory Th1 | lo/+ | + | − | + | + | − | + |
| Transitional memory Th1 | lo/+ | + | − | − | + | − | + |
| Effector memory Th1 | − | − | − | −/+ | −/+ | − | + |
| Terminal effector Th1 | − | − | + | −/+ | −/+ | − | + |
| CD4+ Th2 cells | lo/+ | −/+ | −/+ | −/+ | −/+ | − | − |
| Central memory Th2 | lo/+ | + | − | + | + | − | − |
| Transitional memory Th2 | lo/+ | + | − | − | + | − | − |
| Effector memory Th2 | lo/+ | − | − | −/+ | −/+ | − | − |
| Terminal effector Th2 | lo/+ | − | + | −/+ | −/+ | − | − |
| CD4+ Th17 cells | lo/+ | −/+ | −/+ | −/+ | −/+ | − | − |
| Central memory Th17 | lo/+ | + | − | + | + | − | − |
| Transitional memory Th17 | lo/+ | + | − | − | + | − | − |
| Effector memory Th17 | lo/+ | − | − | −/+ | −/+ | − | − |
| Terminal effector Th17 | lo/+ | − | + | −/+ | −/+ | − | − |
| CD4+ Th1/Th17 cells | lo/+ | −/+ | −/+ | −/+ | −/+ | − | + |
| Central memory Th1/Th17 | lo/+ | + | − | + | + | − | + |
| Transitional memory Th1/Th17 | lo/+ | + | − | − | + | − | + |
| Effector memory Th1/Th17 | lo/+ | − | − | −/+ | −/+ | − | + |
| Terminal effector Th1/Th17 | lo/+ | − | + | −/+ | −/+ | − | + |
| CD4+ Th22 cells | lo/+ | −/+ | −/+ | −/+ | −/+ | − | − |
| Central memory Th22 | lo/+ | + | − | + | + | − | − |
| Transitional memory Th22 | lo/+ | + | − | − | + | − | − |
| Effector memory Th22 | lo/+ | − | − | −/+ | −/+ | − | − |
| Terminal effector Th22 | lo/+ | − | + | −/+ | −/+ | − | − |
| CD4+ CD183+ CD194+ CD196+ CCR10+ | lo/+ | −/+ | −/+ | −/+ | −/+ | − | + |
| CM CD4+ CD183+ CD194+ CD196+ CCR10+ | lo/+ | + | − | + | + | − | + |
| TM CD4+ CD183+ CD194+ CD196+ CCR10+ | lo/+ | + | − | − | + | − | + |
| EM CD4+ CD183+ CD194+ CD196+ CCR10+ | lo/+ | − | − | −/+ | −/+ | − | + |
| TE CD4+ CD183+ CD194+ CD196+ CCR10+ | lo/+ | − | + | −/+ | −/+ | − | + |
| CD4+ CD183+ CD194+ CD196+ CCR10− | lo/+ | −/+ | −/+ | −/+ | −/+ | − | + |
| CM CD4+ CD183+ CD194+ CD196+ CCR10− | lo/+ | + | − | + | + | − | + |
| TM CD4+ CD183+ CD194+ CD196+ CCR10− | lo/+ | + | − | − | + | − | + |
| EM CD4+ CD183+ CD194+ CD196+ CCR10− | lo/+ | − | − | −/+ | −/+ | − | + |
| TE CD4+ CD183+ CD194+ CD196+ CCR10− | lo/+ | − | + | −/+ | −/+ | − | + |
| CD4+ CD183+ CD194+ CD196− CCR10+ | lo/+ | −/+ | −/+ | −/+ | −/+ | − | + |
| CM CD4+ CD183+ CD194+ CD196− CCR10+ | lo/+ | + | − | + | + | − | + |
| TM CD4+ CD183+ CD194+ CD196− CCR10+ | lo/+ | + | − | − | + | − | + |
| EM CD4+ CD183+ CD194+ CD196− CCR10+ | lo/+ | − | − | −/+ | −/+ | − | + |
| TE CD4+ CD183+ CD194+ CD196− CCR10+ | lo/+ | − | + | −/+ | −/+ | − | + |
| CD4+ CD183+ CD194+ CD196− CCR10− | lo/+ | −/+ | −/+ | −/+ | −/+ | − | + |
| CM CD4+ CD183+ CD194+ CD196− CCR10− | lo/+ | + | − | + | + | − | + |
| TM CD4+ CD183+ CD194+ CD196− CCR10− | lo/+ | + | − | − | + | − | + |
| EM CD4+ CD183+ CD194+ CD196− CCR10− | lo/+ | − | − | −/+ | −/+ | − | + |
| TE CD4+ CD183+ CD194+ CD196− CCR10− | lo/+ | − | + | −/+ | −/+ | − | + |
| CD4+ CD183+ CD194− CD196+ CCR10+ | lo/+ | −/+ | −/+ | −/+ | −/+ | − | + |
| CM CD4+ CD183+ CD194− CD196+ CCR10+ | lo/+ | + | − | + | + | − | + |
| TM CD4+ CD183+ CD194− CD196+ CCR10− | lo/+ | + | − | − | + | − | + |
| EM CD4+ CD183+ CD194− CD196+ CCR10+ | lo/+ | − | − | −/+ | −/+ | − | + |
| TE CD4+ CD183+ CD194− CD196+ CCR10+ | lo/+ | − | + | −/+ | −/+ | − | + |
| CD4+ CD183+ CD194− CD196− CCR10+ | lo/+ | −/+ | −/+ | −/+ | −/+ | − | + |
| CM CD4+ CD183+ CD194− CD196− CCR10+ | lo/+ | + | − | + | + | − | + |
| TM CD4+ CD183+ CD194− CD196− CCR10+ | lo/+ | + | − | − | + | − | + |
| EM CD4+ CD183+ CD194− CD196− CCR10+ | lo/+ | − | − | −/+ | −/+ | − | + |
| TE CD4+ CD183+ CD194− CD196− CCR10+ | lo/+ | − | + | −/+ | −/+ | − | + |
| CD4+ CD183− CD194− CD196+ CCR10− | lo/+ | −/+ | −/+ | −/+ | −/+ | − | − |
| CM CD4+ CD183− CD194− CD196+ CCR10− | lo/+ | + | − | + | + | − | − |
| TM CD4+ CD183− CD194− CD196+ CCR10− | lo/+ | + | − | − | + | − | − |
| EM CD4+ CD183− CD194− CD196+ CCR10− | lo/+ | − | − | −/+ | −/+ | − | − |
| TE CD4+ CD183− CD194− CD196+ CCR10− | lo/+ | − | + | −/+ | −/+ | − | − |
| CD4+ CD183− CD194+ CD196− CCR10+ | lo/+ | −/+ | −/+ | −/+ | −/+ | − | − |
| CM CD4+ CD183− CD194+ CD196− CCR10+ | lo/+ | + | − | + | + | − | − |
| TM CD4+ CD183− CD194+ CD196− CCR10+ | lo/+ | + | − | − | + | − | − |
| EM CD4+ CD183− CD194+ CD196− CCR10+ | lo/+ | − | − | −/+ | −/+ | − | − |
| TE CD4+ CD183− CD194+ CD196− CCR10+ | lo/+ | − | + | −/+ | −/+ | − | − |
| non-naïve CD4+ CD183− CD194− CD196− CCR10− | lo/+ | −/+ | − | −/+ | −/+ | − | − |
| CD4+ Tregs | +hi | −/+ | −/+ | −/+ | −/lo | − | −/+ |
| Naïve Treg | +hi | + | + | + | +/lo | − | − |
| Th1-like Treg | +hi | −/+ | −/+ | −/+ | −/lo | − | + |
| CM Th1-like Treg | +hi | + | − | + | +/lo | − | + |
| TM Th1-like Treg | +hi | + | − | − | +/lo | − | + |
| EM Th1-like Treg | +hi | − | − | −/+ | − | − | + |
| TE Th1-like Treg | +hi | − | + | −/+ | − | − | + |
| Th2-like Treg | +hi | −/+ | −/+ | −/+ | −/lo | − | − |
| CM Th2-like Treg | +hi | + | − | + | +/lo | − | − |
| TM Th2-like Treg | +hi | + | − | − | +/lo | − | − |
| EM Th2-like Treg | +hi | − | − | −/+ | − | − | − |
| TE Th2-like Treg | +hi | − | + | −/+ | − | − | − |
| Th17-like Treg | +hi | −/+ | −/+ | −/+ | −/lo | − | − |
| CM Th17-like Treg | +hi | + | − | + | +/lo | − | − |
| TM Th17-like Treg | +hi | + | − | − | +/lo | − | − |
| EM Th17-like Treg | +hi | − | − | −/+ | − | − | − |
| TE Th17-like Treg | +hi | − | + | −/+ | − | − | − |
| Th22-like Treg | +hi | −/+ | −/+ | −/+ | −/lo | − | − |
| CM Th22-like Treg | +hi | + | − | + | +/lo | − | − |
| TM Th22-like Treg | +hi | + | − | − | +/lo | − | − |
| EM Th22-like Treg | +hi | − | − | −/+ | − | − | − |
| TE Th22-like Treg | +hi | − | + | −/+ | − | − | − |
| CD183+ CD194+ CD196− CCR10+ Treg | +hi | −/+ | −/+ | −/+ | −/lo | − | + |
| CM CD183+ CD194+ CD196− CCR10+ Treg | +hi | + | − | + | +/lo | − | + |
| TM CD183+ CD194+ CD196− CCR10+ Treg | +hi | + | − | − | +/lo | − | + |
| EM CD183+ CD194+ CD196− CCR10+ Treg | +hi | − | − | −/+ | − | − | + |
| TE CD183+ CD194+ CD196− CCR10+ Treg | +hi | − | + | −/+ | − | − | + |
| CD183+ CD194+ CD196− CCR10− Treg | +hi | −/+ | −/+ | −/+ | −/lo | − | + |
| CM CD183+ CD194+ CD196− CCR10− Treg | +hi | + | − | + | +/lo | − | + |
| TM CD183+ CD194+ CD196− CCR10− Treg | +hi | + | − | − | +/lo | − | + |
| EM CD183+ CD194+ CD196− CCR10− Treg | +hi | − | − | −/+ | − | − | + |
| TE CD183+ CD194+ CD196− CCR10− Treg | +hi | − | + | −/+ | − | − | + |
| CD183+ CD194+ CD196+ CCR10− Treg | +hi | −/+ | −/+ | −/+ | −/lo | − | + |
| CM CD183+ CD194+ CD196+ CCR10− Treg | +hi | + | − | + | +/lo | − | + |
| TM CD183+ CD194+ CD196+ CCR10− Treg | +hi | + | − | − | +/lo | − | + |
| EM CD183+ CD194+ CD196+ CCR10− Treg | +hi | − | − | −/+ | − | − | + |
| TE CD183+ CD194+ CD196+ CCR10− Treg | +hi | − | + | −/+ | − | − | + |
| CD183+ CD194+ CD196+ CCR10+ Treg | +hi | −/+ | −/+ | −/+ | −/lo | − | + |
| CM CD183+ CD194+ CD196+ CCR10+ Treg | +hi | + | − | + | +/lo | − | + |
| TM CD183+ CD194+ CD196+ CCR10+ Treg | +hi | + | − | − | +/lo | − | + |
| EM CD183+ CD194+ CD196+ CCR10+ Treg | +hi | − | − | −/+ | − | − | + |
| TE CD183+ CD194+ CD196+ CCR10+ Treg | +hi | − | + | −/+ | − | − | + |
| CD183− CD194+ CD196− CCR10+ Treg | +hi | −/+ | −/+ | −/+ | −/lo | − | − |
| CM CD183− CD194+ CD196− CCR10+ Treg | +hi | + | − | + | +/lo | − | − |
| TM CD183− CD194+ CD196− CCR10+ Treg | +hi | + | − | − | +/lo | − | − |
| EM CD183− CD194+ CD196− CCR10+ Treg | +hi | − | − | −/+ | − | − | − |
| TE CD183− CD194+ CD196− CCR10+ Treg | +hi | − | + | −/+ | − | − | − |
| CD4+ Tfh cells | −/lo | −/+ | −/+ | −/+ | −/+ | − | −/+ |
| Naïve Tfh | −/lo | + | + | + | + | − | − |
| Treg Tfh | −/lo | −/+ | −/+ | −/+ | −/lo | − | −/+ |
| CM Treg Tfh | −/lo | + | − | + | +/lo | − | −/+ |
| TM Treg Tfh | −/lo | + | − | − | +/lo | − | −/+ |
| EM Treg Tfh | −/lo | − | − | −/+ | − | − | −/+ |
| TE Treg Tfh | −/lo | − | + | −/+ | − | − | −/+ |
| Th1-like Tfh | −/lo | −/+ | −/+ | −/+ | −/+ | − | + |
| CM Th1-like Tfh | −/lo | + | − | + | + | − | + |
| TM Th1-like Tfh | −/lo | + | − | − | + | − | + |
| EM Th1-like Tfh | −/lo | − | − | −/+ | −/+ | − | + |
| TE Th1-like Tfh | −/lo | − | + | −/+ | −/+ | − | + |
| Th2-like Tfh | −/lo | −/+ | −/+ | −/+ | −/+ | − | − |
| CM Th2-like Tfh | −/lo | + | − | + | + | − | − |
| TM Th2-like Tfh | −/lo | + | − | − | + | − | − |
| EM Th2-like Tfh | −/lo | − | − | −/+ | −/+ | − | − |
| TE Th2-like Tfh | −/lo | − | + | −/+ | −/+ | − | − |
| Th17-like Tfh | −/lo | −/+ | −/+ | −/+ | −/+ | − | − |
| CM Th17-like Tfh | −/lo | + | − | + | + | − | − |
| TM Th17-like Tfh | −/lo | + | − | − | + | − | − |
| EM Th17-like Tfh | −/lo | − | − | −/+ | −/+ | − | − |
| TE Th17-like Tfh | −/lo | − | + | −/+ | −/+ | − | − |
| Th1/Th17-like Tfh | −/lo | −/+ | −/+ | −/+ | −/+ | − | + |
| CM Th1/Th17-like Tfh | −/lo | + | − | + | + | − | + |
| TM Th1/Th17-like Tfh | −/lo | + | − | − | + | − | + |
| EM Th1/Th17-like Tfh | −/lo | − | − | −/+ | −/+ | − | + |
| TE Th1/Th17-like Tfh | −/lo | − | + | −/+ | −/+ | − | + |
| CD183+ CD194+ CD196− CCR10− Tfh | −/lo | −/+ | −/+ | −/+ | −/+ | − | + |
| CM CD183+ CD194+ CD196− CCR10− Tfh | −/lo | + | − | + | + | − | + |
| TM CD183+ CD194+ CD196− CCR10− Tfh | −/lo | + | − | − | + | − | + |
| EM CD183+ CD194+ CD196− CCR10− Tfh | −/lo | − | − | −/+ | −/+ | − | + |
| TE CD183+ CD194+ CD196− CCR10− Tfh | −/lo | − | + | −/+ | −/+ | − | + |
| CD1834+ CD194+ CD196+ CCR10− Tfh | −/lo | −/+ | −/+ | −/+ | −/+ | − | + |
| CM CD183+ CD194+ CD196+ CCR10− Tfh | −/lo | + | − | + | + | − | + |
| TM CD183+ CD194+ CD196+ CCR10− Tfh | −/lo | + | − | − | + | − | + |
| EM CD183+ CD194+ CD196+ CCR10− Tfh | −/lo | − | − | −/+ | −/+ | − | + |
| TE CD183+ CD194+ CD196+ CCR10− Tfh | −/lo | − | + | −/+ | −/+ | − | + |
| CD183− CD194− CM96+ CCR10− Tfh | −/lo | −/+ | −/+ | −/+ | −/+ | − | − |
| CM CD183− CD194− CD196+ CCR10− Tf | −/lo | + | − | + | + | − | − |
| TM CD183− CD194− CD196+ CCR10− Tfh | −/lo | + | − | − | + | − | − |
| EM CD183− CD194− CD196+ CCR10− Tfh | −/lo | − | − | −/+ | −/+ | − | − |
| TE CD183− CD194− CD196+ CCR10− Tfh | −/lo | − | + | −/+ | −/+ | − | − |
| CD183− CD194− CD196− CCR10− Tfh | −/lo | −/+ | −/+ | −/+ | −/+ | − | − |
| CM CD183− CD194− CD196− CCR10− Tfh | −/lo | + | − | + | + | − | − |
| TM CD183− CD194− CD196− CCR10− Tfh | −/lo | + | − | − | + | − | − |
| EM CD183− CD194− CD196− CCR10− Tfh | −/lo | − | − | −/+ | −/+ | − | − |
| TE CD183− CD194− CD196− CCR10− Tfh | −/lo | − | + | −/+ | −/+ | − | − |

| | | | | | Frequency |
|---|---|---|---|---|---|
| Peripheral blood CD4+ T-Cell populations | | | | | (from total |
| (CD3+CD4+CD45++) | CD185 | CD194 | CD196 | CCR10 | CD4+ T cells) |
| CD4+ naïve T cells | − | − | − | − | >10% |
| CD4+ Th1 cells | − | − | − | − | >10% |
| Central memory Th1 | − | − | − | − | 5-10% |
| Transitional memory Th1 | − | − | − | − | 1-5% |
| Effector memory Th1 | − | − | − | − | 1-5% |
| Terminal effector Th1 | − | − | − | − | <0.5% |
| CD4+ Th2 cells | − | + | − | − | 1-5% |
| Central memory Th2 | − | + | − | − | 1-5% |
| Transitional memory Th2 | − | + | − | − | <0.5% |
| Effector memory Th2 | − | + | − | − | <0.5% |
| Terminal effector Th2 | − | + | − | − | <0.5% |
| CD4+ Th17 cells | − | + | + | − | 1-5% |
| Central memory Th17 | − | + | + | − | 1-5% |
| Transitional memory Th17 | − | + | + | − | 0.5-1% |
| Effector memory Th17 | − | + | + | − | <0.5% |
| Terminal effector Th17 | − | + | + | − | <0.5% |
| CD4+ Th1/Th17 cells | − | − | + | − | 1-5% |
| Central memory Th1/Th17 | − | − | + | − | 1-5% |
| Transitional memory Th1/Th17 | − | − | + | − | 1-5% |
| Effector memory Th1/Th17 | − | − | + | − | 0.5-1% |
| Terminal effector Th1/Th17 | − | − | + | − | <0.5% |
| CD4+ Th22 cells | − | + | + | + | 1-5% |
| Central memory Th22 | − | + | + | + | <0.5% |
| Transitional memory Th22 | − | + | + | + | 0.5-1% |
| Effector memory Th22 | − | + | + | + | 0.5-1% |
| Terminal effector Th22 | − | + | + | + | <0.5% |
| CD4+ CD183+ CD194+ CD196+ CCR10+ | − | + | + | + | 1-5% |
| CM CD4+ CD183+ CD194+ CD196+ CCR10+ | − | + | + | + | <0.5% |
| TM CD4+ CD183+ CD194+ CD196+ CCR10+ | − | + | + | + | 0.5-1% |
| EM CD4+ CD183+ CD194+ CD196+ CCR10+ | − | + | + | + | <0.5% |
| TE CD4+ CD183+ CD194+ CD196+ CCR10+ | − | + | + | + | <0.5% |
| CD4+ CD183+ CD194+ CD196+ CCR10− | − | + | + | − | 5-10% |
| CM CD4+ CD183+ CD194+ CD196+ CCR10− | − | + | + | − | 1-5% |
| TM CD4+ CD183+ CD194+ CD196+ CCR10− | − | + | + | − | 1-5% |
| EM CD4+ CD183+ CD194+ CD196+ CCR10− | − | + | + | − | 0.5-1% |
| TE CD4+ CD183+ CD194+ CD196+ CCR10− | − | + | + | − | <0.5% |
| CD4+ CD183+ CD194+ CD196− CCR10+ | − | + | − | + | 0.5-1% |
| CM CD4+ CD183+ CD194+ CD196− CCR10+ | − | + | − | + | <0.5% |
| TM CD4+ CD183+ CD194+ CD196− CCR10+ | − | + | − | + | <0.5% |
| EM CD4+ CD183+ CD194+ CD196− CCR10+ | − | + | − | + | <0.5% |
| TE CD4+ CD183+ CD194+ CD196− CCR10+ | − | + | − | + | <0.5% |
| CD4+ CD183+ CD194+ CD196− CCR10− | − | + | − | − | 5-10% |
| CM CD4+ CD183+ CD194+ CD196− CCR10− | − | + | − | − | 1-5% |
| TM CD4+ CD183+ CD194+ CD196− CCR10− | − | + | − | − | 1-5% |
| EM CD4+ CD183+ CD194+ CD196− CCR10− | − | + | − | − | 0.5-1% |
| TE CD4+ CD183+ CD194+ CD196− CCR10− | − | + | − | − | <0.5% |
| CD4+ CD183+ CD194− CD196+ CCR10+ | − | − | + | + | <0.5% |
| CM CD4+ CD183+ CD194− CD196+ CCR10+ | − | − | + | + | <0.5% |
| TM CD4+ CD183+ CD194− CD196+ CCR10− | − | − | + | + | <0.5% |
| EM CD4+ CD183+ CD194− CD196+ CCR10+ | − | − | + | + | <0.5% |
| TE CD4+ CD183+ CD194− CD196+ CCR10+ | − | − | + | + | <0.5% |
| CD4+ CD183+ CD194− CD196− CCR10+ | − | − | − | + | <0.5% |
| CM CD4+ CD183+ CD194− CD196− CCR10+ | − | − | − | + | <0.5% |
| TM CD4+ CD183+ CD194− CD196− CCR10+ | − | − | − | + | <0.5% |
| EM CD4+ CD183+ CD194− CD196− CCR10+ | − | − | − | + | <0.5% |
| TE CD4+ CD183+ CD194− CD196− CCR10+ | − | − | − | + | <0.5% |
| CD4+ CD183− CD194− CD196+ CCR10− | − | − | + | − | 0.5-1% |
| CM CD4+ CD183− CD194− CD196+ CCR10− | − | − | + | − | 0.5-1% |
| TM CD4+ CD183− CD194− CD196+ CCR10− | − | − | + | − | <0.5% |
| EM CD4+ CD183− CD194− CD196+ CCR10− | − | − | + | − | <0.5% |
| TE CD4+ CD183− CD194− CD196+ CCR10− | − | − | + | − | <0.5% |
| CD4+ CD183− CD194+ CD196− CCR10+ | − | + | − | + | 0.5-1% |
| CM CD4+ CD183− CD194+ CD196− CCR10+ | − | + | − | + | <0.5% |
| TM CD4+ CD183− CD194+ CD196− CCR10+ | − | + | − | + | <0.5% |
| EM CD4+ CD183− CD194+ CD196− CCR10+ | − | + | − | + | <0.5% |
| TE CD4+ CD183− CD194+ CD196− CCR10+ | − | + | − | + | <0.5% |
| non-naïve CD4+ CD183− CD194− CD196− CCR10− | − | − | − | − | 0.5-1% |
| CD4+ Tregs | −/+ | −/+ | −/+ | −/+ | 5-10% |
| Naïve Treg | − | − | − | − | <0.5% |
| Th1-like Treg | − | − | − | − | <0.5% |
| CM Th1-like Treg | − | − | − | − | <0.5% |
| TM Th1-like Treg | − | − | − | − | <0.5% |
| EM Th1-like Treg | − | − | − | − | <0.5% |
| TE Th1-like Treg | − | − | − | − | <0.5% |
| Th2-like Treg | − | + | − | − | 0.5-1% |
| CM Th2-like Treg | − | + | − | − | <0.5% |
| TM Th2-like Treg | − | + | − | − | <0.5% |
| EM Th2-like Treg | − | + | − | − | <0.5% |
| TE Th2-like Treg | − | + | − | − | <0.5% |
| Th17-like Treg | − | + | + | − | 0.5-1% |
| CM Th17-like Treg | − | + | + | − | <0.5% |
| TM Th17-like Treg | − | + | + | − | <0.5% |
| EM Th17-like Treg | − | + | + | − | <0.5% |
| TE Th17-like Treg | − | + | + | − | <0.5% |
| Th22-like Treg | − | + | + | + | 1-5% |
| CM Th22-like Treg | − | + | + | + | <0.5% |
| TM Th22-like Treg | − | + | + | + | <0.5% |
| EM Th22-like Treg | − | + | + | + | <0.5% |
| TE Th22-like Treg | − | + | + | + | <0.5% |
| CD183+ CD194+ CD196− CCR10+ Treg | − | + | − | + | <0.5% |
| CM CD183+ CD194+ CD196− CCR10+ Treg | − | + | − | + | <0.5% |
| TM CD183+ CD194+ CD196− CCR10+ Treg | − | + | − | + | <0.5% |
| EM CD183+ CD194+ CD196− CCR10+ Treg | − | + | − | + | <0.5% |
| TE CD183+ CD194+ CD196− CCR10+ Treg | − | + | − | + | <0.5% |
| CD183+ CD194+ CD196− CCR10− Treg | − | + | − | − | <0.5% |
| CM CD183+ CD194+ CD196− CCR10− Treg | − | + | − | − | <0.5% |
| TM CD183+ CD194+ CD196− CCR10− Treg | − | + | − | − | <0.5% |
| EM CD183+ CD194+ CD196− CCR10− Treg | − | + | − | − | <0.5% |
| TE CD183+ CD194+ CD196− CCR10− Treg | − | + | − | − | <0.5% |
| CD183+ CD194+ CD196+ CCR10− Treg | − | + | + | − | 0.5-1% |
| CM CD183+ CD194+ CD196+ CCR10− Treg | − | + | + | − | <0.5% |
| TM CD183+ CD194+ CD196+ CCR10− Treg | − | + | + | − | <0.5% |
| EM CD183+ CD194+ CD196+ CCR10− Treg | − | + | + | − | <0.5% |
| TE CD183+ CD194+ CD196+ CCR10− Treg | − | + | + | − | <0.5% |
| CD183+ CD194+ CD196+ CCR10+ Treg | − | + | + | + | 0.5-1% |
| CM CD183+ CD194+ CD196+ CCR10+ Treg | − | + | + | + | <0.5% |
| TM CD183+ CD194+ CD196+ CCR10+ Treg | − | + | + | + | <0.5% |
| EM CD183+ CD194+ CD196+ CCR10+ Treg | − | + | + | + | <0.5% |
| TE CD183+ CD194+ CD196+ CCR10+ Treg | − | + | + | + | <0.5% |
| CD183− CD194+ CD196− CCR10+ Treg | − | + | − | + | <0.5% |
| CM CD183− CD194+ CD196− CCR10+ Treg | − | + | − | + | <0.5% |
| TM CD183− CD194+ CD196− CCR10+ Treg | − | + | − | + | <0.5% |
| EM CD183− CD194+ CD196− CCR10+ Treg | − | + | − | + | <0.5% |
| TE CD183− CD194+ CD196− CCR10+ Treg | − | + | − | + | <0.5% |
| CD4+ Tfh cells | + | −/+ | −/+ | − | >10% |
| Naïve Tfh | + | − | − | − | 0.5-1% |
| Treg Tfh | + | −/+ | −/+ | − | 1-5% |
| CM Treg Tfh | + | −/+ | −/+ | − | 0.5-1% |
| TM Treg Tfh | + | −/+ | −/+ | − | 0.5-1% |
| EM Treg Tfh | + | −/+ | −/+ | − | <0.5% |
| TE Treg Tfh | + | −/+ | −/+ | − | <0.5% |
| Th1-like Tfh | + | − | − | − | 1-5% |
| CM Th1-like Tfh | + | − | − | − | 0.5-1% |
| TM Th1-like Tfh | + | − | − | − | 0.5-1% |
| EM Th1-like Tfh | + | − | − | − | <0.5% |
| TE Th1-like Tfh | + | − | − | − | <0.5% |
| Th2-like Tfh | + | + | − | − | 1-5% |
| CM Th2-like Tfh | + | + | − | − | 0.5-1% |
| TM Th2-like Tfh | + | + | − | − | 0.5-1% |
| EM Th2-like Tfh | + | + | − | − | <0.5% |
| TE Th2-like Tfh | + | + | − | − | <0.5% |
| Th17-like Tfh | + | + | + | − | 5-10% |
| CM Th17-like Tfh | + | + | + | − | 0.5-1% |
| TM Th17-like Tfh | + | + | + | − | 0.5-1% |
| EM Th17-like Tfh | + | + | + | − | <0.5% |
| TE Th17-like Tfh | + | + | + | − | <0.5% |
| Th1/Th17-like Tfh | + | − | + | − | 1-5% |
| CM Th1/Th17-like Tfh | + | − | + | − | 0.5-1% |
| TM Th1/Th17-like Tfh | + | − | + | − | 0.5-1% |
| EM Th1/Th17-like Tfh | + | − | + | − | <0.5% |
| TE Th1/Th17-like Tfh | + | − | + | − | <0.5% |
| CD183+ CD194+ CD196− CCR10− Tfh | + | + | − | − | 1-5% |
| CM CD183+ CD194+ CD196− CCR10− Tfh | + | + | − | − | 0.5-1% |
| TM CD183+ CD194+ CD196− CCR10− Tfh | + | + | − | − | 0.5-1% |
| EM CD183+ CD194+ CD196− CCR10− Tfh | + | + | − | − | <0.5% |
| TE CD183+ CD194+ CD196− CCR10− Tfh | + | + | − | − | <0.5% |
| CD1834+ CD194+ CD196+ CCR10− Tfh | + | + | + | − | 1-5% |
| CM CD183+ CD194+ CD196+ CCR10− Tfh | + | + | + | − | 0.5-1% |
| TM CD183+ CD194+ CD196+ CCR10− Tfh | + | + | + | − | 0.5-1% |
| EM CD183+ CD194+ CD196+ CCR10− Tfh | + | + | + | − | <0.5% |
| TE CD183+ CD194+ CD196+ CCR10− Tfh | + | + | + | − | <0.5% |
| CD183− CD194− CM96+ CCR10− Tfh | + | − | + | − | 1-5% |
| CM CD183− CD194− CD196+ CCR10− Tf | + | − | + | − | 0.5-1% |
| TM CD183− CD194− CD196+ CCR10− Tfh | + | − | + | − | 0.5-1% |
| EM CD183− CD194− CD196+ CCR10− Tfh | + | − | + | − | <0.5% |
| TE CD183− CD194− CD196+ CCR10− Tfh | + | − | + | − | <0.5% |
| CD183− CD194− CD196− CCR10− Tfh | + | − | − | − | 1-5% |
| CM CD183− CD194− CD196− CCR10− Tfh | + | − | − | − | 0.5-1% |
| TM CD183− CD194− CD196− CCR10− Tfh | + | − | − | − | 0.5-1% |
| EM CD183− CD194− CD196− CCR10− Tfh | + | − | − | − | <0.5% |
| TE CD183− CD194− CD196− CCR10− Tfh | + | − | − | − | <0.5% |

*CD154 is only expressed by activated T cells
lo: low;
hi: high

TABLE 5
Exemplary Antibody combinations against 12 cell surface markers supplemented with the classical
antibody combination for diagnosis and classification of CD4 T-cell malignancies & the corresponding
tube for the diagnosis and classification of Cytotoxic T-NK cell malignancies, to be merged
and calculated according to the EuroFlow guidelines (www.EuroFlow.org)*, **

| | F1 | F2 | F3 | F4 | F5 | F6 | F7 | F8 | F9 | F10 | F11 | F12 |
|---|---|---|---|---|---|---|---|---|---|---|---|---|
| CD4 | CD27 | CD45RA | CD45RO | CD8 | CD62L | CD127 | CD3 | CD25 | CCR10 | CD183 | CD196 | CD194 |
| | | | | | | | | | | (CXCR3) | (CCR6) | (CCR4) |
| Cytotox | CD27 | CD45RA | CD45RO | CD8 | CD62L | CD127 | CD3 | CD25 | CD11c | CD16 | CD56 | CD57 |

| | F13 | F14 | F15 | F16 | F17 | F18 | F19 | F20 | F21 | F22 |
|---|---|---|---|---|---|---|---|---|---|---|
| CD4 | CD185 | CD2 | CD279 | CD7 | CD26 | CD28 | HLA-DR | cyTCL1 | CD45 | CD4 |
| | (CXCR5) | | | | | | | | | |
| Cytotox | TcRγδ | CyGranB | CyPerforin | CD7 | CD30 | CD94 | HLA-DR | CD5 | CD45 | CD4 |

*The proposed tubes can be further extended with antibodies against TcR-Vβ and/or TcR-Vα domains (Table 6) plus an anti-TcRγδ antibody or with antibodies against TcR-Vδ and/or TcR-Vγ domains plus an anti-TcRαβ antibodies.
**The proposed tubes can be further extended with antibodies against mutually exclusive TcR-Cβ1 and TcR-Cβ2 epitopes, such as the epitope recognized by the JOVI-1 antibody (BD Biosciences) for clonality assessment in TcRαβ+ T-cell malignancies.

### B.2 CD4 T-Cell Tube as Basis for Diagnosis and Classification of Mature T-Cell Malignancies.

In another embodiment, the above 12-marker combination (with both CD45RO and CD45RA and with or without CD45 and/or CD8) can be supplemented with the classical EuroFlow antibody combination of CD2, CD7, CD26, CD28, CD279 optionally in combination with HLA-DR, and/or cyTCL1, for diagnosis and classification of CD4 positive mature T-cell malignancies (Van Dongen et al. 2012) (Table 5). Therefore, the invention also provides a reagent composition comprising conjugated antibodies against CD27, CD45RA or CD45RO, CD62L, CD127, CD3, CD25, CCR10, CD183, CD196, CD194, CD185, CD4, CD2, CD279, CD7, CD26 and CD28, optionally further comprising conjugated antibodies against CD8, CD45, HLA-DR, and/or cyTCL1.

This antibody panel allows for subclassification of the different types of CD4 positive T-cell malignancies according to their T-cell functional subsets of conventional Th1, Th2, Th17, Th22, Th1/Th17 T-cells and CD4 positive Th1-like, Th2-like, Th17-like, Th22-like, Th1/Th17-like Tregs and Tfh T-cells, together with a further subclassification into the 5 maturation stages of naïve, central memory, transitional memory, effector memory and terminal effector CD4 T-cells.

The results of this CD4+ T-cell malignancy tube can typically be merged and calculated with the results of the corresponding “sister tube” for the diagnosis and classification of Cytotoxic T & NK cell malignancies, using the markers CD27, CD45RA, CD45RO, CD8, CD62L, CD127, CD3, CD25, CD7, HLA-DR, CD45 and/or CD4 as backbone marker set (bold in Table 5) according to the EuroFlow guidelines (Kalina et al., 2012), supplemented with the Cytotoxic T & NK cell markers CD5, CD11c, CD16, CD56, CD57, TcRy6, and optionally granzymeB and/or perforin (Table 5). Hence, also provided is a set of reagent compositions, comprising (i) a reagent composition comprising conjugated antibodies against CD27, CD45RA or CD45RO, CD62L, CD127, CD3. CD25, CCR10, CD183, CD196, CD194, CD185, CD4, CD2, CD279, CD7. CD26 and CD28, optionally further comprising conjugated antibodies against CD8, CD45, HLA-DR, and/or cyTCL1; and (ii) a reagent composition comprising conjugated antibodies directed against the markers CD27, CD45RA, CD45RO, CD8, CD62L, CD127, CD3, CD25, CD7, HLA-DR, CD45, CD4, CD11c, CD16, CD30, CD56, CD57, CD94, CD5, TcRy6, optionally supplemented with conjugated antibodies directed against granzyme B and/or perforin (see Table 5). Preferably, within a set of reagents the antibodies being present in both reagent compositions are conjugated to the same detectable label.

### B.3 TcR Repertoire Studies in TcRαβ Cell Populations and TcRγδ Cell Populations

To study the diversity repertoire of the TcR molecules on the TcRαβ cells, the above marker combinations (see section B.1) can be further extended with conjugated antibodies against the various families TcR-V8 domains and/or several families of TcR-Vα domains plus an anti-TcRγδ antibody to recognize the non-TcRαβ cells.

Analogously, to study the diversity of the TcR molecules on TcRγδ cells, the above marker combinations can be further extended with conjugated antibodies against TcR-V8 and/or TcR-Vy domains plus an anti-TcRαβ antibody to recognize the non-TcRγ6 cells. It is also envisaged to use an antibody cocktail comprising conjugated antibodies against the various families TcR-Vβ domains and/or several families of TcR-Vα domains, the TcRγ6, the TcR-Vδ and/or TcR-Vγ domains, plus an anti-TcRαβ antibody.

Most required antibodies against TcR-V8 and TcRα domains are commercially available, such as the IOTest Beta Mark Kit (Beckman Coulter) with 24 antibodies against VB family domains (see also Table 6). Only a few anti-Vα antibodies are available, such as anti-TcR-Vα2 (B20.1), TcR-Vα3.2 (RR3-16), TcR-Vα7.2 (3C10), TcR-Vα11 (RR8-1), TcR-Vα12.1 (6D6.6) and TcR-Vα24-Jα18 (6b11) (ThermoFischer Scientific; and Abcam) (Table 6).

For studying the expression of TcR-Vδ and/or TcR-Vγ domains, a panel of conjugated antibodies can be composed with specificity for V61 (R912 and dTCS1), Vδ2 (IMMU389 and BB3), Vδ3 (P11.5B), and non-Vδ1 (IMMU515) and for Vγ2/3/4 (23D12), Vγ4 (4A11), Vγ3/5 (56.3; this antibody recognizes Vγ5 domains and some Vγ3 domains), Vγ8 (R4.5), and Vγ9 (IMMU360 and Ti-gA). Mab 4A11 is available from T Cell Diagnostics (Woburn, Mass.), dTCS1 from T Cell Sciences (Cambridge, Mass.); Mab 23D12, R4.5, IMMU360, R912, IMMU389, P11.5B, and IMMU515 were obtained from Beckman Coulter/Immunotech. The Ti-gA, BB3, and 56.3 Mabs were kind gifts of Dr. T Hercend (Villejuif, France), Dr. L Moretta (Genova, Italy), and Dr. D Kabelitz (Kiel, Germany), respectively.

In the above embodiments, each antibody against the different TcR-Vδ, TcR-Vα, TcR-Vγ, and TcR-Vδ family domains might be conjugated to a single detectable label (i.e. fluorochrome or metal isotopes) or with two or more (up to nine) distinct labels (i.e. fluorochromes or metal isotopes with separate emissions from each other) to allow for the measurement of all TcR-V specificities in a condensed number of fluorescence or metal isotope detectors, e.g. 30 antibodies combined with a total of at least 5 detectable labels with distinct emissions for the TcR-Vs antibodies and 3 labels for the TcR-Vα antibodies, as exemplified in Table 6.

The addition of the above anti-TcR-V antibodies to the above mentioned reagent compositions for diagnosis and classification of mature T-cell malignancies allows for assessing clonality of the involved T-cell malignancies (Langerak et al., 1999; Langerak et al., 2001; Lima et al. 2001; Almeida et al. 2003, Sandberg et al. 2006). Such clonality markers can further support the monitoring of T-cell malignancies during and after treatment to asses treatment effectiveness.

TABLE 6
Example of staining with 24 Vβ antibodies conjugated
to only five detectable labels (F1-F5) and 6 Vα
antibodies conjugated to only three detectable labels (F6-F8)

| | Gene | | | | | | | | | |
|---|---|---|---|---|---|---|---|---|---|---|
| No | segment | Clone* | F1 | F2 | F3 | F4 | F5 | F6 | F7 | F8 |
| B1 | Vβ1 | BL37.2 | X | | | | | | | |
| B2 | Vβ2 | MPB2D5 | | X | | | | | | |
| B3 | Vβ3 | CH92 | | | X | | | | | |
| B4 | Vβ4 | WJF24 | | | | X | | | | |
| B5 | Vβ5.1 | IMMU157 | | | | | X | | | |
| B6 | Vβ5.2 | 36213 | X | X | | | | | | |
| B7 | Vβ5.3 | 3D11 | X | | X | | | | | |
| B8 | Vβ7.1 | ZOE | X | | | X | | | | |
| B9 | Vβ7.2 | ZIZOU4 | X | | | | X | | | |
| B10 | Vβ8 | 56C5.2 | | X | X | | | | | |
| B11 | Vβ9 | FIN9 | | X | | X | | | | |
| B12 | Vβ11 | C21 | | X | | | X | | | |
| B13 | Vβ12 | VER2.32 | | | X | X | | | | |
| B14 | Vβ13.1 | IMMU222 | | | X | | X | | | |
| B15 | Vβ13.2 | H132 | | | | X | X | | | |
| B16 | Vβ13.6 | JU74.3 | X | X | X | | | | | |
| B17 | Vβ14 | CAS1.1.3 | X | X | | X | | | | |
| B18 | Vβ16 | TAMAYA1.2 | X | X | | | X | | | |
| B19 | Vβ17 | E17.5F3 | X | | X | X | | | | |
| B20 | Vβ18 | BA62.6 | X | | X | | X | | | |
| B21 | Vβ20 | ELL1.4 | X | | | X | X | | | |
| B22 | Vβ21.3 | IG125 | | X | X | X | | | | |
| B23 | Vβ22 | IMMU546 | | X | X | | X | | | |
| B24 | Vβ23 | AF23 | | X | | X | X | | | |
| A1 | Vα2 | B20.1 | | | | | | X | | |
| A2 | Vα3.2 | RR3-16 | | | | | | | X | |
| A3 | Vα7.2 | 3C10 | | | | | | | | X |
| A4 | Vα11 | RR8-1 | | | | | | X | X | |
| A5 | Vα12.1 | 6D6.6 | | | | | | X | | X |
| A6 | Vα24- | 6B11 | | | | | | | X | X |
| | Jα18 | | | | | | | | | |

*Antibodies against Vβ and Vα domains available from Beckman Coulter, ThermoFisher Scientific and Abcam

B.4 TcR-Cβ1 vs. TcR-Cβ2 Expression Pattern as Surrogate Markers for Clonality in T-Cell Populations

For decades, the exclusive expression of the Igκ and Igλ proteins by individual B-cells has been used as a surrogate marker for detection of clonality in B-cell populations, thereby identifying clonal B-cell expansions and consequently supporting the diagnosis of mature B-cell malignancies, including myeloma. Where B-cells produce antibodies that can associate either Igκ proteins (derived from a functionally rearranged IGK locus on chromosome 2) or Igλ proteins (derived from a functionally rearranged IGL gene on chromosome 22), T-cells do not have such mutually exclusive usage of two different genes.

However, the TcR-Cβ domain (Constant domain of the TcRβ chain) can be derived from the TCRBC1 exon or the TCRBC2 exon, dependent on whether the expressed TCRB rearrangement involves Jβ1 gene segments or Jβ2 gene segments. This alternative (mutually exclusive) usage of the TcR-Cβ1 vs. TcR-C82 protein domains is indeed identified in both normal and malignant T-cell populations, but has never been regarded as a potential source for clonality studies, albeit that this alternative TcR-Cβ1 vs. TcR-Cβ2 expression is quite stable at the T-cell population level, varying from 40% to 60%.

Interestingly, whereas the two D-J-C gene sets in the TCRB locus are evolutionary duplications and are therefore highly homologous, a few clear differences have been identified in the TCRBC1 exon vs. the TCRBC2 exon. At amino acid position 4 and 5, the N and K amino acids of TcR-Cβ1 domain are replaced by the K and N amino acids in the TcR-Cβ2 domain and the F amino acid at position 37 in the TcR-Cβ1 domain is replaced by the Y amino acid in the TcR-Cβ2 domain. Additional amino acid differences are present in the membrane-proximal sequences, where a V amino acid is replaced by an E amino acid and an F amino acid by an S amino acid in the TcR-Cβ2 domain. The first two amino acid differences (positions 4-5 and position 37) are closely linked in the folded TcRβ chains, so that they form a unique epitope, potentially suited for differential recognition by antibodies. So far no specific antibodies against both such different TcR-C61 and TcR-Cβ2 epitopes have been designed. However, the reactivity of the JOVI-1 antibody (BD Biosciences) against a combined rearranged human TCR-Vδ3-C61 chain appears to be mainly restricted to T cells expressing TcR-C81 domains. Therefore, the present invention provides the insight that the ratio of JOVI-1 positive vs. JOVI-1 negative T-cells is advantageously used as a surrogate marker for alternative TcR-C61 vs. TcR-Cβ2 domain expression, and thereby a surrogate marker for the detection of clonality in TcRαB+ T-cells. Consequently, such alternative TcR-Cβ1 vs. TcR-Cβ2 domain expression can be used for identifying clonal TcRαB+ T-cell expansions and consequently supporting the diagnosis of mature TcRαB+ T-cell malignancies. This alternative TcR-C61 vs. TcR-Cβ2 domain expression can be applied to define diversity (e.g. “normal” TcR-Cβ1/TcR-Cβ2 ratio) in the TcRαB+ T-cell compartment, particularly in TcRαB+ T-cells that are not recognized by the antibodies against TcR-Vβ and/or TcR-Vα domains (Table 6).

### C.1 Plasmablast/Plasma Cell & B-Cell Tube

Circulating plasmablasts are currently defined on the basis of marker CD38 alone or in combination with CD19 and/or CD27 (Medina et al, 2002; Odendahl et al, 2005; Clutterbuck et al, Immunology 2006; Mei et al, 2009; Caraux et al, 2010; Perez-Andres et al, 2010; Quian et al, 2010; Maecker et al, 2012; Roederer et al., 2015; Blanco, 2017; Blanco et al, 2018; Blanco et al, 2019; Liechti et al, 2019).

However, the combination of these markers together does not allow the identification of all blood plasmablasts in children. Blood plasmablasts have been shown to have heterogeneous expression of CD20, CD62L and CD138 with both negativity and positivity of these markers. Combinations of CD20 with CD138 have defined three maturation subsets of blood plasmablasts: CD20+/CD138⁻, CD20⁻, CD138⁻, and CD20−/CD138+(Perez-Andres et al, 2010). In addition, CD62L has also revealed two maturation subsets of plasmablasts in blood (Mei et al, 2009). However, no one has combined the three markers to establish the relationship between these plasmablast subsets as defined by CD20, CD62L, and CD138.

By combining the markers CD20, CD38, CD62L and CD138, optionally with CD19 (Table 7), the present inventors were to first to observe that the above mentioned plasmablast subsets are not discrete subsets, but together form a full continuous maturation pathway from CD19+CD20+CD38+CD62L-CD138- to mature plasma cells (CD19+CD20-CD38++CD62L+CD138+(FIG. 6). This is assumed to be an identical distribution over all the different isotypes.

Accordingly, in one embodiment the invention provides a reagent composition for the cytometric immunophenotyping of leukocytes comprising antibodies conjugated to a detectable label, the conjugated antibodies being directed against the following combination of markers: CD20, CD38, CD62L, and CD138, optionally combined with CD19.

Recently we have shown that B-lineage cells can be subdivided according to the expression of the IgH classes and subclasses (isotypes) in combination or not with Ig kappa/lambda (Igκ and Igλ) light chains (Blanco, 2017; Blanco et al, 2018; Blanco et al, 2019). Nobody has taught how to investigate differences in distribution of the individual IgH isotypes throughout the plasmablast/plasma cell maturation pathway in blood or other tissues (Table 7; see also population tree in FIG. 7).

The four marker-based (with or without CD19) plasmablast/plasma cell pathway reagent composition in combination with IgH class and/or subclass evaluation is a novel tool for blood monitoring of ongoing B-cell responses in tissues throughout the body and for the first time permits accurate clear cut identification of all plasmablasts/plasma cells in blood of both adults and children. As illustrated in FIGS. 6B and D, this further allows detailed dissection of B-cell responses against individual microbiome, ongoing infections, allergic responses, auto-immunity, vaccines and immunotherapy.

In one embodiment, any of the above antibody panels for plasmablast/plasma cells and B-cells is supplemented with a reagent that allows for detecting antigen-specific plasmablast/plasma cells and B-cells, for example micro-organism-specific, allergen-specific, auto-antigen specific, and vaccine-specific plasmablast/plasma cells and B-cells and/or antigen-specific plasmablast/plasma cells and B-cells in the setting of immunotherapy. Such antigens for staining antigen-specific plasmablast/plasma cells and B-cells can be directly conjugated with a label, indirectly detected by a second step labeled reagent or linked to a labeled multimer system known in the art, such as the Klickmer and Dextramer systems (Immudex, Copenhagen, Denmark).

The marker combination for plasmablast/plasma cell maturation pathway analysis per IgH compartment consists of a combination of CD38, CD62L, CD20, CD138, plus IgM, with or without CD19. In addition, IgA plus IgG can be added with the same or with two different labelings, one for IgA and one for IgG.

In another embodiment, a conjugated antibody against IgD can be added with the same labeling as antibodies against IgG and IgA, or a different labeling to that of both IgA and IgG. Alternatively, antibodies against IgD, IgG and IgA are combined in 2 labelings in such a way that one of the three reagents is conjugated simultaneously with the two labelings and the other 2 reagents are combined with only one of those two labelings each.

In another version of the antibody combination, as cited above, IgA and IgG and IgD antibodies are replaced with Igκ and Igλ reagents, both conjugated to the same label or each to a distinct label.

In another embodiment, the CD38, CD62L, CD20, CD138 backbone antibodies with or without IgM and with or without CD19, can be combined with both cell surface staining and intracellular staining for Igκ and Igλ, preferably wherein different labelings are used for the cell surface membrane and the intracellular reagents, and the same or different labellings are used for the Igκ and the Igλ reagents to allow accurate identification of blood plasmablasts/plasma cells, particularly in children (Table 7). In addition, the above mentioned antibody combinations can be supplemented with antibodies against the different IgH isotypes and IgA and/or IgG subclasses, as described above for the BB0 backbone (see footnote in Table 7). In another embodiment, IgE antibodies conjugated with one or simultaneously two labelings may be added to each of the above antibody combinations (Table 7).

Any of these marker combinations can be supplemented with directly or indirectly labeled micro-organism antigens, auto-antigens, vaccine-antigens, allergens, or immunotherapy associated components.

TABLE 7
Exemplary Antibody combinations for the Plasmablast/plasma cell & B-cell tubes*, **

| | | | | | | | | F8 e.g. | | F10 e.g. | | | | |
|---|---|---|---|---|---|---|---|---|---|---|---|---|---|---|
| | F1 e.g. | F2 e.g. | F3 e.g. | F4 e.g. | F5 e.g. | F6 e.g. | F7 e.g. | PerCP | F9 e.g. | PE | F11 e.g. | F12 e.g. | F13 e.g. | F14 e.g. |
| | BV421 | BV510 | BV605 | BV650 | BV711 | BV786 | FITC | Cy5.5 | PE | CF594 | PE Cy7 | APC | AF700 | APC H7 |
| BB0A | | | CD62L | | | w/wo | | | | CD20 | CD138 | | | CD38 |
| | | | | | | CD19 | | | | | | | | |
| BB0B | cyIgM | | CD62L | | | w/wo | | | | CD20 | CD138 | | | CD38 |
| | | | | | | CD19 | | | | | | | | |
| BB0C | smIgk | | CD62L | | | w/wo | cyIgk | | | CD20 | CD138 | | | CD38 |
| | smIgλ | | | | | CD19 | cyIgλ | | | | | | | |
| BB0D | smIgk | | CD62L | | | w/wo | cyIgk | cyIgλ | | CD20 | CD138 | | | CD38 |
| | smIgλ | | | | | CD19 | | | | | | | | |
| BB1A | cyIgM | | CD62L | | | w/wo | cyIgA, | | | CD20 | CD138 | | | CD38 |
| | | | | | | CD19 | cyIgG | | | | | | | |
| BB1B | cyIgM | | CD62L | | | w/wo | cyIgA | cyIgG | | CD20 | CD138 | | | CD38 |
| | | | | | | CD19 | | | | | | | | |
| BB1C | cyIgM | | CD62L | | | w/wo | cyIgD, | | | CD20 | CD138 | | | CD38 |
| | | | | | | CD19 | cyIgA | | | | | | | |
| | | | | | | | cyIgG | | | | | | | |
| BB1D | cyIgM | | CD62L | | | w/wo | cyIgD | cyIgA | cyIgG | CD20 | CD138 | | | CD38 |
| | | | | | | CD19 | | | | | | | | |
| BB1E | cyIgM | | CD62L | | | w/wo | cyIgD | cyIgG | | CD20 | CD138 | | | CD38 |
| | | | | | | CD19 | cyIgA | CyIgA | | | | | | |
| BB1F | cyIgM | | CD62L | | | w/wo | cyIgD | CyIgD | | CD20 | CD138 | | | CD38 |
| | | | | | | CD19 | cyIgA | cyIgG | | | | | | |
| BB1G | cyIgM | | CD62L | | | w/wo | cyIgG | cyIgG | | CD20 | CD138 | | | CD38 |
| | | | | | | CD19 | cyIgA | cyIgD | | | | | | |
| BB1H | cyIgM | CD27 | CD62L | | | w/wo | cyIgG | cyIgG | | CD20 | CD138 | | | CD38 |
| | | | | | | CD19 | cyIgA | cyIgD | | | CD5 | | | |
| BB2 | cyIgM | | CD62L | | | w/wo | cyIgκ, | | | CD20 | CD138 | | | CD38 |
| | | | | | | CD19 | cyIgλ | | | | | | | |
| BB2A | cyIgM | | CD62L | | | w/wo | cyIgκ | cyIgλ | | CD20 | CD138 | | | CD38 |
| | | | | | | CD19 | | | | | | | | |
| BB3 | cyIgM | | CD62L | | | w/wo | cyIgG3 | cyIgA1 | cyIgG1 | CD20 | CD138 | cyIgA1 | | CD38 |
| | | | | | | CD19 | cyIgG2 | cyIgA2 | cyIgG2 | | | cyIgG4 | | |
| | | | | | | | cyIgD | | | | | cyIgD | | |
| BB3A | cyIgM | | | | | w/wo | cyIgG3 | cyIgA1 | cyIgG1 | CD20 | CD138 | cyIgA1 | | CD38 |
| | | | | | | CD19 | cyIgG2 | cyIgA2 | cyIgG2 | | | cyIgG4 | | |
| | | | | | | | cyIgD | cyIgE | cyIgE | | | cvIgD | | |
| BB4 | cyIgM | CD27 | CD62L | | | | cyIgG3 | cyIgA1 | cyIgG1 | | CD5 | cyIgA1 | w/wo | CD38 |
| | | | | | | | cyIgG2 | cyIgA2 | cyIgG2 | | CD138 | cyIgG4 | CD45 | |
| | | | | | | | cyIgD | cyIgE | cyIgE | | | cyIgD | | |
| BB4A | cyIgM | CD27 | CD62L | | | CD19 | cyIgG3 | cyIgA1 | cyIgG1 | CD20 | CD5 | cyIgA1 | w/wo | CD38 |
| | | | | | | | cyIgG2 | cyIgA2 | cyIgG2 | | CD138 | cyIgG4 | CD45 | |
| | | | | | | | cyIgD | cyIgE | cyIgE | | | cyIgD | | |
| BB5 | cyIgM | CD27 | CD62L | CD24 | CD21 | CD19 | cyIgG3 | cyIgA1 | cyIgG1 | CD20 | | cyIgA1 | | |
| | | | | | | | cyIgG2 | cyIgA2 | cyIgG2 | | | cyIgG4 | | |
| | | | | | | | cyIgD | cyIgE | cyIgE | | | cyIgD | | |
| BB5A | cyIgM | CD27 | CD62L | CD24 | CD21 | CD19 | IgD | IgD | IgD | CD20 | | | | |
| | | | | | | | IgG2 | IgG1 | IgG1 | | | | | |
| | | | | | | | IgA1 | IgA1 | IgG2 | | | | | |
| | | | | | | | IgG3 | IgA2 | IgG4 | | | | | |

*The BB1 tubes can be further completed with Igkappa and Iglambda staining on separate labeling positions.
*From tube BB3 onwards, Igkappa and Iglambda antibodies can be added on separate labeling positions. Alternatively, the BB0B and BB0C can be combined with cyIgM and the additional reagents contained in backbones BB1 to BB5A, except BB2 and BB2A.
**Immunoglobulin staining of all previous tubes can be done only intracellularly, only surface membrane or both intracellular and surface membrane. The later one could be done with the markers on the same labeling position or separate labeling positions.

D: Admixtures of Antibodies for Combined Detection of DC-Monocytes, CD4+ T-Cell Subsets, and/or Plasmablast/Plasma Cell & B-Cells

The invention also provides means and methods for advanced leukocyte/immune cell subsetting using an admixture (i.e. a cocktail) of two or three backbone (BB) marker combinations of the invention, which define the main subpopulations per cell lineage and maturation compartment.

For example, based on the advances in flow cytometry with more than 40 different (fluorochrome) colors, and mass cytometry with more than 40 different (metal) colors, it has now become possible use a single reagent composition comprising a combination of the above mentioned 15 BB marker combination of the DC-monocyte tube with the 12 BB markers of the CD4 T-cell tube and/or the 5 BB markers of the Plasmablast/plasma cell & B-cell tube:
DC-Monocyte BB (15 markers): CD5, CD14, CD16, CD33, CD34, CD36, CD45, CD62L, CD141, CD192, CD300e, CD303, FcER1, HLA-DR, and SLAN;
CD4+ T-cell BB (12 markers): CD3, CD4, CD25, CD27, CD45RA, CD62L, CD127, CD183, CD185, CD194, CD196, and CCR10 with or without CD45
Plasmablast/plasma cell & B-cell BB (5 markers): CD20, CD38, CD62L, CD138, and IgM or Igk plus Igλ with or without CD19

These three BB marker sets are not only novel but also unique, because they are highly complementary with very limited overlap (only CD62L and CD45), implying that two (DC-monocyte BB and CD4+ T cell BB, or DC-monocyte BB and Plasmablast/plasma cell & B cell BB or CD4+ T cell BB and Plasmablast/plasma cell & B cell BB) or even three (DC-monocyte BB and CD4+ T cell BB and Plasmablast/plasma cell & B cell BB) BB markers sets can be combined to create new unique (extensive) marker combinations.

The three BB sets together provide a highly informative novel antibody combination, which allows advanced multi-lineage leukocyte/immune cell subsetting. Data collection using such 30-marker (28-color) antibody combination (with or without CD19) is currently feasible with several types of cytometers, such as Symphony-A5 (≥35-colors/fluorochromes; BD Biosciences, San José, CA), Aurora-5L (≥35-colors/fluorochromes; Cytek, Freemont, Calif.), and CyTOF Helios (≥40-colors/metals; Fluidigm, South San Francisco, Calif.).

These BB antibody admixtures can be further extended with the extra markers, as defined above for further dissection of the individual leukocyte/immune cell lineages. For example, adding antibodies against CD8, CD19, CD56, TCRγ6, and the IgH isotypes (IgH classes or subclasses) creates the possibility to detect and quantify more than 350 subsets of blood leukocytes in a single tube.

In another embodiment, any of the sets of backbone markers or combinations of backbone markers is combined with markers devoted to detect circulating tumor cells in blood or minimal residual disease in bone marrow in both hematological malignancies and other types of cancer, and/or with markers devoted to monitor genetically modified immune cells, such as CAR-T and CAR-NK cells, including any type of CAR-T or CAR NK cells such as CAR-CD19, CAR-BCMA. CAR-CD30, CAR-CD20, CAR-EGFR, etc.

### D. Condensing the Antibody Panels to 12-Color Tubes for Analysis of DC-Monocytes, CD4+ T-Cells, and Plasmablast/Plasma Cell & B-Cells

Whereas advanced ≥25-color flow cytometers are progressively more available in many research and clinical laboratories, many diagnostic laboratories still use 8-12-color stainings in routine diagnostic patient care. Therefore, the above presented reagent compositions should preferably also be available for patient care around the world. To achieve such world-wide applicability, several of the above antibody panels have been adapted into 12-color reagent compositions. For doing so, combinations of markers (cellular targets) have been identified that are mutually exclusive, i.e. are never expressed on the same cell, so that the corresponding antibodies can be conjugated to the same detectable label.

For example, CD34 and CD14 are never expressed simultaneously on the same normal DC-monocytic cell population. Antibodies against CD34 and CD14 can therefore be conjugated to the same fluorochrome, implying that the CD5, CD14, CD16, CD33, CD34, CD36, CD45, CD62L, CD141, CD192, CD300e, CD303, FcER1, HLA-DR, and SLAN antibody combination can be “condensed” into a 12-color, 15-antibody DC-monocyte tube (Table 8).

The invention therefore provides a 12-color reagent composition comprising conjugated antibodies against CD141, HLA-DR, CD16, CD33, CD300e, CD303, CD14, CD45, CD5. CD34, CD36, SLAN, CD62L, FcERI and CD192(CCR2), wherein the antibodies within the marker sets SLAN/FcERI, CD300e/CD303, and CD14/CD34 are conjugated to the same label and wherein between the different marker sets the labels are distinguishable.

Alternatively, markers with clearly different expression levels and different staining patterns can be combined, and the corresponding antibodies can be conjugated with the same label. For example, CD4 and CD45 differ significantly in expression pattern and expression level and can therefore be combined in the same label position in the CD4 T-cell tube, implying that the CD3, CD4, CD25, CD27, CD45, CD62L, CD127, CD183, CD185, CD194, CD196, CCR10, and CD45RA or CD45RO markers result into a 12-color, 13 antibody combination (Table 8).

The invention therefore provides a 12-color reagent composition comprising conjugated antibodies against CD27, CD62L, CD127, CD3, CD25, CCR10, CD183 (CXCR3), CD196 (CCR6), CD194 (CCR4), CD185 (CXCR5), CD4, CD45, and CD45RA or CD45RO, wherein the antibodies against CD4 and CD45 are conjugated to the same label (12-color CD4 T cell tube).

The results of this 12-color CD4 T-cell tube can typically be merged and calculated with the results of the corresponding “sister tube” for Cytotoxic T & NK cells, using the 6 markers CD3, CD4, CD27, CD45, CD62L, and CD45RA or CD45RO as backbone marker set (bold in Table 8) according to the EuroFlow guidelines (Kalina et al., 2012) (www.EuroFlow.org). This Cytotoxic T & NK cell tube further comprises antibodies against CD8, CD16, CD28, CD56, CD57, TcRγδ, and granzyme B (Table 8). Accordingly, also provided is a set of 12-color reagent compositions, comprising (i) the reagent composition as defined for the 12-color CD4 T cell tube; and (ii) a 12-color reagent composition comprising conjugated antibodies directed against the markers CD27, CD62L, CD3, CD45, CD4, CD45RO or CD45RA, CD8, CD16, CD28, CD56, CD57, TcRγδ, and granzyme-B, wherein the antibodies against CD4 and CD45 are conjugated to the same label. Within a reagent set, each composition should contain an antibody against either CD45RO or CD45RA.

Finally, the invention provides Plasmablast/plasma cell & B-cell tube composed of the backbone markers CD19, CD20, CD38, CD62L and CD138, supplemented with CD27, CD21, CD45, IgM and IgD. This antibody composition may be further supplemented with antibodies against Igκ and IgH, or against IgG and IgA, resulting in a 12-color reagent composition which allows detailed immune monitoring of the mature B-lineage compartment (Table 8).

Except for IgM and IgD, all other IgH isotypes are not expressed simultaneously by the same B-lineage cell, implying that several combinations can be made of corresponding antibodies conjugated to the same detectable label. Consequently, in another embodiment the Plasmablast/plasma cell & B-cell reagent composition contains 14 different antibodies, one of which is present in the form of two different fluorochrome conjugates (anti-IgD, anti-IgG, or anti-IgA), while Igκ and anti-Igλ are positioned at the same fluorochrome, implying that 15 different antibody conjugates are present in this 12-color tube. In yet another embodiment, the 12-color Plasmablast/plasma cell & B-cell tube is composed of 16 different antibodies, three of which are present in two different fluorochrome conjugates (e.g. anti-IgG1, anti-IgG2, and anti-IgA1) while another anti-IgH antibody (e.g. anti-IgD) is present in the form of three different conjugates, implying that 21 different antibody conjugates are present in this 12-color tube (Table 8).

### EXAMPLE

It will be appreciated by a person skilled in the art of cytometry and immunophenotyping that each of the reagent compositions as described herein above can be applied in routine procedures and protocols for sample preparation and cytometric analysis.
This Example merely illustrates how exemplary reagent compositions can be used for the analysis of distinct populations of cells in a biological sample.

For all multiparameter flow cytometric immunophenotyping studies, fresh samples were used, such as fresh blood, bone marrow (BM), lymph node and spleen specimens. These samples were stained and processed within 24 h after collection. For blood and BM samples, the NH₄Cl-based EuroFlow bulk-lyse standard operating procedure (SOP) was used to lyse non-nucleated red cells prior to staining (Flores Montero et al 2017), except for the CD4 T-cell immunostaining tube, in which a direct immunofluorescence stain-and-then-lyse technique was used (Kalina et al. 2012)). Lymph node and spleen samples were mechanically dissociated into single cell suspensions, prior to staining. Afterward, at least 5×10⁶ leukocytes per sample aliquot (or at least 3×10⁵ cells/tube for the CD4 T-cell immunostaining tube) were stained with the appropriate series of (monoclonal) antibody reagents.

Acquisition of the stained samples was performed immediately after immunostaining, in an LSR Fortessa X-20 flow cytometer (BD), using the FACSDiva™ software (BD). For instrument setup, calibration and monitoring, the EuroFlow instrument setup & compensation protocol for 14-color measurements was used. For data analysis, the Infinicyt™ software (Cytognos S.L) was employed. For each blood sample, relative and absolute (double-platform) cell counts were calculated and recorded for both the major cell population of interest and each specific cell subset. In BM, lymph node and spleen samples, only relative cell numbers were derived.

With reference to FIG. 1, in one of the dendritic cell (DC)-monocyte immunostaining tubes, the following 15 Mab reagents were used: CD141 (1A4)-Brilliant Violet (BV) 421 (BD Biosciences), CD5 (UCHT2)-BV510 (BD Biosciences), CD192 (LS132.1D9)-BV605 (BD Biosciences), CD62L (DREG-56)-BV650 (BioLegend), anti-HLA-DR (G46-6)-BV711 (BD Biosciences), CD16 (3G8)-BV786 (BD Biosciences), CD36 (CLB-IVC7)-peridinin chlorophyll protein-cyanine 5.5 (PerCPCy5.5) (Immunostep), anti-SLAN (DD-1)-phycoerythrin (PE) (Miltenyi), anti-FcERI (AER-37)-PE (Thermo Fisher), CD34 (581)-PECF594 (BD Biosciences), CD33 (P67.6)-PECy7 (BD Biosciences), CD300e (UP-H2)-allophycocyanine (APC) (Immunostep), CD303 (AC144)-APC (Miltenyi), CD45 (HI30)-AF700 (BD Biosciences), and CD14 (MφpP9)-APCH7 (BD Biosciences). The anti-SLAN and anti-FcER1 antibodies can both be conjugated to PE, because the expression of SLAN and FcER1 on monocyte subsets are mutually exclusive. The same applies to the APC-conjugated CD300e (IREM2) and APC-conjugated CD303 antibodies, because the expression of CD300e and CD303 are mutually exclusive. This allows for the application of 16 different antibodies, using 14 different detectable labels. With reference to FIG. 4, and as a distinct example, in one of the CD4 T-cell immunostaining tubes, the following 14 Mab reagents were used: CD27 (M-T271)-BV421 (BD Biosciences), CD45RA (HI100) BV510 (BD Biosciences), CD8 (SK1)-BV605 (BD Biosciences), CD62L (DREG-56)-BV650 (BioLegend), CD127 (HIL7RM21)-BV711) BD Biosciences), CD3 (SK7)-BV786 (BD Biosciences), CD25 (4E3)-VioBright fluorescein isothiocyanate (FITC) (Miltenyi), CCR10 (1B5)-PerCPCy5.5 (BD Biosciences), CD183-CXCR3 (1C6/CXCR3)-PE (BD Biosciences), CD196-CCR6 (11A9)-PECF594) (BD Biosciences), CD194-:30 CCR4 (L291H4)-PECy7 (BioLegend), CD185-CXCR5 (REA103)-APC (Miltenyi), CD45 (HI30)-AF700, and CD4 (SK3)-APCH7 (BD Biosciences).

TABLE 8
Exemplary 12-color Reagent Compositions for Immune Monitoring

| | | | | | | F6 | F7 |
|---|---|---|---|---|---|---|---|
| | F1 | F2 | F3 | F4 | F5 | e.g. | e.g. |
| | e.g. | e.g. | e.g. | e.g. | e.g. | FITC or | PerCP |
| | BV421 | BV510 | BV605 | BV711 | BV786 | BB515 | Cy5.5 |
| DC/ | CD141 | CD5 | CD62L | HLA-DR | CD16 | CD192 | CD36 |
| Monocyte | | | | | | | |
| CD4 T | CD27 | CD45RA | CD62L | CD127 | CD3 | CD25 | CCR10 |
| CD4 T | CD27 | CD45RO | CD62L | CD127 | CD3 | CD25 | CCR10 |
| Cytotox T | CD27 | CD45RA | CD62L | CD16 | CD3 | CD57 | CD28 |
| & NK cells | | | | | | | |
| Cytotox T | CD27 | CD45RO | CD62L | CD16 | CD3 | CD57 | CD28 |
| & NK cells | | | | | | | |
| Plasmablast/ | CD27 | IgM | CD62L | CD21 | CD19 | IgD | Igκ |
| plasma cell | | | | | | | |
| & B-cell | | | | | | | |
| Plasmablast/ | CD27 | IgM | CD62L | CD21 | CD19 | IgD | IgG |
| plasma cell | | | | | | | |
| & B-cell | | | | | | | |
| Plasmablast/ | CD27 | IgM | CD62L | CD21 | CD19 | IgD | IgD |
| plasma cell | | | | | | IgG | IgA |
| & B-cell | | | | | | | |
| Plasmablast/ | CD27 | IgM | CD62L | CD21 | CD19 | IgD | IgD |
| plasma cell | | | | | | IgG2 | IgG1 |
| & B-cell | | | | | | IgA1 | IgA1 |
| | | | | | | IgG3 | IgA2 |

| | | | | F11 | F12 | |
|---|---|---|---|---|---|---|
| | F8 | F9 | F10 | e.g. | e.g. | |
| | e.g. | e.g. | e.g. | AF700 or | APC-H7 or | No. of Ab |
| | PE | PE Cy7 | APC | APC-R700 | APC-Cy7 | (different) |
| DC/ | SLAN | CD33 | CD300e | CD45 | CD14 + | 15 |
| Monocyte | FcERI | | CD303 | | CD34 | |
| CD4 T | CD183 | CD194 | CD185 | CD196 | CD4 + | 13 |
| | (CXCR3) | (CCR4) | (CXCR5) | (CCR6) | CD45 | |
| CD4 T | CD183 | CD194 | CD185 | CD196 | CD4 + | 13 |
| | (CXCR3) | (CCR4) | (CXCR5) | (CCR6) | CD45 | |
| Cytotox T | cyGranB | TcRγδ | CD8 | CD56 | CD45 + | 13 |
| & NK cells | | | | | CD4 | |
| Cytotox T | cyGranB | TcRγδ | CD8 | CD56 | CD45 + | 13 |
| & NK cells | | | | | CD4 | |
| Plasmablast/ | Igλ | CD138 | CD20 | CD45 | CD38 | 12 |
| plasma cell | | | | | | |
| & B-cell | | | | | | |
| Plasmablast/ | IgA | CD138 | CD20 | CD45 | CD38 | 12 |
| plasma cell | | | | | | |
| & B-cell | | | | | | |
| Plasmablast/ | Igκ | CD138 | CD20 | CD45 | CD38 | 15* |
| plasma cell | Igλ | | | | | (14) |
| & B-cell | | | | | | |
| Plasmablast/ | IgD | CD138 | CD20 | CD45 | CD38 | 21** |
| plasma cell | IgG1 | | | | | (16) |
| & B-cell | IgG2 | | | | | |
| | IgG4 | | | | | |

* The Plasmablast/plasma cell & B-cell tube can contain 14 different antibodies, one of which is present in the form of two different fluorochrome conjugates (anti-IgD, anti-IgG, or anti-IgA), while Igκ and anti-Igλ are positioned at the same fluorochrome, implying that 15 different antibody conjugates are present in this 12-color tube.
**The Plasmablast/plasma cell & B-cell tube can contain 16 different antibodies, three of which are present in two different fluorochrome conjugates (anti-IgG1, anti-IgG2, and anti-IgA1) and anti-IgD is present in the form of three different conjugates, implying that 21 different antibody conjugates are present in this 12-color tube.

### REFERENCES

Alcantara-Hernandez, M. R. Leylek, L. E. Wagar, E. G. Engleman, T. Keler, M. P. Marinkovich, M. M. Davis, G. P. Nolan and J. Idoyaga (2017). “High-Dimensional Phenotypic Mapping of Human Dendritic Cells Reveals Interindividual Variation and Tissue Specialization.” Immunity 47(6): 1037-1050 e1036.
Appay, V., van Lier, R. A. W., Sallusto, F., Roederer, M., 2008. Phenotype and function of human T lymphocyte subsets: Consensus and issues. Cytom. Part A 73A, 975-983. https://doi.org/10.1002/cyto.a.20643
Autissier, P. C. Soulas, T. H. Burdo and K. C. Williams (2010). “Evaluation of a 12-color flow cytometry panel to study lymphocyte, monocyte, and dendritic cell subsets in humans.” Cytometry A 77(5): 410-419.
Bachem, A. S. Guttler, E. Hartung, F. Ebstein, M. Schaefer, A. Tannert, A. Salama. K. Movassaghi, C. Opitz, H. W. Mages, V. Henn, P. M. Kloetzel, S. Gurka and R. A. Kroczek (2010). “Superior antigen cross-presentation and XCRI expression define human CD11c+CD141+ cells as homologues of mouse CD8+ dendritic cells.” J Exp Med 207(6): 1273-1281.
Blanco, E., Perez-Andres, M., Sanoja-Flores, L., Wentink, M., Pelak, O., Martin-Ayuso, M., Grigore, G., Torres-Canizales, J., López-Granados, E., Kalina, T., van der Burg, M., Arriba-Mendez, S., Santa Cruz, S., Puig, N., van Dongen, J J M., Orfao A., 2017. Selection and validation of antibody clones against IgG and IgA subclasses in switched memory B-cells and plasma cells. J Immunol Methods.
Blanco, E., Pérez-Andrés, M., Arriba-Méndez, S., Contreras-Sanfeliciano, T., Criado. I., Pelak, O., Serra-Caetano, A., Romero, A., Puig, N., Remesal, A., Torres Canizales. J., López-Granados, E., Kalina, T., Sousa, A E., van Zelm, M., van der Burg, M., van Dongen, J J M., Orfao, A.; EuroFlow PID group. 2018. Age-associated distribution of normal B-cell and plasma cell subsets in peripheral blood. J Allergy Clin Immunol. 141, 2208-2219.e16.
Blanco, E., Pérez-Andrés, M., Arriba-Méndez, S., Serrano, C., Criado, I., Del Pino-Molina, L., Silva, S., Madruga, I., Bakardjieva, M., Martins, C., Serra-Caetano, A., Romero, A., Contreras-Sanfeliciano, T., Bonroy, C., Sala, F., Martin, A., Bastida, J M., Lorente, F., Prieto, C., Dávila, I., Marcos, M., Kalina, T., Vlkova, M., Chovancova, Z., Cordeiro. A I., Philippé, J., Haerynck, F., López-Granados, E., Sousa, A E., van der Burg, M., van Dongen, J J M., Orfao, A.; EuroFlow PID group. 2019. Defects in memory B-cell and plasma cell subsets expressing different immunoglobulin-subclasses in patients with CVID and immunoglobulin subclass deficiencies. J Allergy Clin Immunol.
Brodie, T. M., Tosevski, V., 2018. Broad immune monitoring and profiling of T cell subsets with mass cytometry, in: Methods in Molecular Biology. pp. 67-82. https://doi.org/10.1007/978-1-4939-7680-5_4
Caraux, A., Klein, B., Paiva, B., Bret, C., Schmitz, A., Fuhler, G M., Bos, N A., Johnsen, H E., Orfao, A., Perez-Andres, M., Myeloma Stem Cell Network, 2010. Circulating human B and plasma cells. Age-associated changes in counts and detailed characterization of circulating normal CD138- and CD138+ plasma cells. Haematologica 95, 1016-20. https://doi.org/10.3324/haematol.2009.018689.
Clutterbuck, E A., Salt, P., Oh, S., Marchant, A., Beverley, P., Pollard, A J., 2006. The kinetics and phenotype of the human B-cell response following immunization with a heptavalent pneumococcal-CRM conjugate vaccine. Immunology I19.328-37. https://doi.org/10.1111/j.1365-2567.2006.02436.x
Collin, M. and V. Bigley (2018). “Human dendritic cell subsets: an update.” Immunology 154(1): 3-20.
De Rosa, S. C., Carter, D. K., McElrath, M. J., 2012. OMIP-014: Validated multifunctional characterization of antigen-specific human T cells by intracellular cytokine staining. Cytom. Part A 81A, 1019-1021. https://doi.org/10.1002/cyto.a.22218
Draxler, D. F., M. T. Madondo, G. Hanafi, M. Plebanski and R. L. Medcalf (2017). “A flowcytometric analysis to efficiently quantify multiple innate immune cells and T Cell subsets in human blood.” Cytometry A 91(4): 336-350.
Duhen, T., Duhen, R., Lanzavecchia, A., Sallusto, F., Campbell, D. J., 2012. Functionally distinct subsets of human FOXP3+ Treg cells that phenotypically mirror effector Th cells. Blood 119, 4430-4440. https://doi.org/10.1182/blood-2011-11-392324
Finak, G., Langweiler, M., Jaimes, M., Malek, M., Taghiyar, J., Korin, Y., Raddassi, K., Devine, L., Obermoser, G., Pekalski, M. L., Pontikos, N., Diaz, A., Heck, S., Villanova, F., Terrazzini, N., Kern, F., Qian, Y., Stanton, R., Wang, K., Brandes, A., Ramey, J., Aghaeepour, N., Mosmann, T., Scheuermann, R. H., Reed, E., Palucka, K., Pascual, V., Blomberg, B. B., Nestle, F., Nussenblatt, R. B., Brinkman, R. R., Gottardo, R., Maecker, H., McCoy, J. P., 2016. Standardizing Flow Cytometry Immunophenotyping Analysis from the Human ImmunoPhenotyping Consortium. Sci. Rep. 6, 20686. https://doi.org/10.1038/srep20686
Flores-Montero J, Sanoja-Flores L, Paiva B, Puig N, Garcia-Sánchez O, Bottcher S, van der Velden V H J, Pérez-Morán J J, Vidriales M B, Garcia-Sanz R, Jimenez C, González M. Martinez-López J, Corral-Mateos A. Grigore G E, Fluxá R, Pontes R, Caetano J. Sedek L, Del Cañizo M C, Bladé J, Lahuerta J J, Aguilar C, Bárez A. García-Mateo A. Labrador J, Leoz P, Aguilera-Sanz C, San-Miguel J, Mateos M V. Durie B, van Dongen J J M, Orfao A. 2017. Next Generation Flow for highly sensitive and standardized detection of minimal residual disease in multiple myeloma. Leukemia 31:2094-2103.
Fromm, P. D., F. Kupresanin, A. E. Brooks, P. R. Dunbar, M. Ilaniffa, D. N. Hart and G, J. Clark (2016). “A multi-laboratory comparison of blood dendritic cell populations.” Clin Transl Immunology 5(4): e68.
Gattinoni, L. Lugli, E., Ji, Y., Pos, Z., Paulos, C. M., Quigley, M. F., Almeida, J, R., Gostick, E., Yu, Z., Carpenito, C., Wang, E., Douek, D. C., Price, D. A., June, C, H., Marincola, F. M., Roederer, M., Restifo, N. P., 2011. A Human Memory T Cell Subset with Stem Cell-like Properties. Nature Medicine 17 (10): 1290-97. https://doi.org/10.1038/nm.2446
Hasan, M. B. Beitz, V. Rouilly, V. Libri, A. Urrutia, D. Duffy, L. Cassard, J. P. Di Santo, E. Mottez, L. Quintana-Murci, M. L. Albert, L. Rogge and C. Milieu Interieur (2015). “Semi-automated and standardized cytometric procedures for multi-panel and multi-parametric whole blood immunophenotyping.” Clin Immunol 157(2): 261-276.
Heinze, A. M, C. Elze, S. Kloess, O. Ciocarlie, C. Konigs, S. Betz, M. Bremm, R. Esser, T. Klingebiel, M. Serban, J. L. Hutton and U. Koehl (2013). “Age-matched dendritic cell subpopulations reference values in childhood.” Scand J Immunol 77(3): 21:3-220.
Kalina T. Flores-Montero J, Van der Velden V H J, Martin-Ayuso M. Böttcher S. Ritgen M, Almeida J, Lhermitte L. Asnafi V, Mendonça A, de Tute R. Cullen M. Sedek L, Vidriales M B, Pérez J J, Te Marvelde J G, Mejstrikova E, Hrusak O. Szczepański T. Van Dongen J J M, Orfao A., EuroFlow Consortium (EU-FP6, LSHB-CT-2006-018708), 2012. EuroFlow standardization of flow cytometer instrument settings and immunophenotyping protocols. Leukemia 26, 1986-2010.
Kohler, S., Thiel, A., 2009. Life after the Thymus: CD31+ and CD31− Human Naive CD4+ T-Cell Subsets. Blood 113 (4): 769-74. https://doi.org/10.1182/blood-2008-02-139154.
Kunicki, M. A., Amaya Hernandez, L. C., Davis, K. L., Bacchetta, R., Roncarolo, M.-G., 2018. Identity and Diversity of Human Peripheral Th and T Regulatory Cells Defined by Single-Cell Mass Cytometry, J. Immunol. 200. 3:36-:346. https://doi.org/10.4049/jimmunol.1701025
Langerak A W, Wolvers-Tettero I L M, Van den Beemd M W M, Van Wering E R, Ludwig W-D, Hählen K, Necker A, Van Dongen J J M, 1999. Immunophenotypic and immunogenotypic characteristics of TCRγδ+ T cell acute lymphoblastic leukemia. Leukemia 13, 206-214.
Langerak A W, Van den Beemd R, Wolvers-Tettero I L, Boor P P, Van Lochem E G, Hooijkaas H, Van Dongen J J M, 2001. Molecular and flow cytometric analysis of the VB repertoire for clonality assessment in mature TCRaB T-cell proliferations. Blood 98.165-173.
Larbi, A., Fulop, T., 2014. From “truly naïve” to “exhausted senescent” T cells: when markers predict functionality. Cytometry A 85, 25-35. https://doi.org/10.1002/cyto.a.22351
Liecht, i T., Roederer, M., 2019. OMIP-051-28-color flow cytometry panel to characterize B cells and myeloid cells. Cytometry A. 95, 150-155. https://doi.org/10.1002/cyto.a.23689.
Lima M, Almeida J. Santos A H, dos Anjos Teixeira M, Alguero M C, Queirós ML, Balanzategui A, Justiça B, Gonzalez M, San Miguel J F, Orfão A, 2001. Immunophenotypic analysis of the TCR-Vbeta repertoire in 98 persistent expansions of CD3(+)/TCR-alphabeta(+) large granular lymphocytes: utility in assessing clonality and insights into the pathogenesis of the disease. Am J Pathol. 159:1861-1868.
Almeida J, Dos Anjos Teixeira M. Del Carmen Alguero M, Santos A H, Balanzategui A, Luis Queirós M. Bárcena P, Izarra A, Fonseca S, Bueno C. Justiça B, Gonzalez M, San Miguel J F, Orfao A. 2003. TCRαβ+/CD4+ Large Granular Lymphocytosis, A New Clonal T-Cell Lymphoproliferative Disorder. Am J Pathol 163(2), 763-771. Maecker, H T, McCoy, J P. Nussenblatt, R, 2012. Standardizing immunophenotyping for the Human Immunology Project. Nat Rev Immunol. 12, 191-200. https://doi.org/10.10:38/nri3158.
Mahnke, Yolanda D., Beddall, M. H., Roederer, M., 2013. OMIP-017: Human CD4+ helper T-cell subsets including follicular helper cells. Cytom. Part A 83A. 439-440. https://doi.org/10.1002/cyto.a.22269
Mahnke, Yolanda D. Brodie, T. M., Sallusto, F., Roederer, M., Lugli, E., 2013. The who's who of T-cell differentiation: Human memory T-cell subsets. Eur, J. Immunol. https://doi.org/10.1002/eji.201343751
McAdam, A. J., Greenwald, R. J., Levin, M. A., Chernova, T., Malenkovich, N., Ling, V., Freeman, G. J., Sharpe, A. H., 2001. ICOS is critical for CD40-mediated antibody class switching. Nature 409(6816):102-5. https://doi.org/10.1038/35051107
Medina, F., Segundo, C., Campos-Caro, A., González-García, I., Brieva, J A., 2002. The heterogeneity shown by human plasma cells from tonsil, blood, and bone marrow reveals graded stages of increasing maturity, but local profiles of adhesion molecule expression. Blood 99, 2154-2161. https://doi.org/10.8/blood.v99.6.2154
Mei, H E., Yoshida, T., Sime, W., Hiepe, F., Thiele, K., Manz, R A., Radbruch, A., Dörner, T., 2009. Blood-borne human plasma cells in steady state are derived from mucosal immune responses. Blood 113, 2461-2469. https://doi.org/10.1182/blood-2008-04-153544
Moncunill, G., Dobaño, C., McElrath, M. J., De Rosa, S. C., 2015. OMIP-025: Evaluation of human T- and NK-cell responses including memory and follicular helper phenotype by intracellular cytokine staining. Cytom. Part A 87, 289-292. https://doi.org/10.1002/cyto.a.22590
Morita, R., Schmitt, N., Bentebibel, S.-E., Ranganathan, R., Bourdery, L., Zurawski, G., Foucat, E., Dullaers, M., Oh, S., Sabzghabaei, N., Lavecchio, E. M., Punaro, M., Pascual, V., Banchereau, J., Ueno, 11., 2011. Human blood CXCR5(+)CD4(+) T cells are counterparts of T follicular cells and contain specific subsets that differentially support antibody secretion. Immunity 34, 108-21. https://doi.org/10.1016/j.immuni.2010.12.012
Mousset, C. M., Hobo, W., Woestenenk, R., Preijers, F., Dolstra, H., van der Waart., A, B., 2019. Comprehensive Phenotyping of T Cells Using Flow Cytometry. Cytometry, A. https://doi.org/10.100′2/cyto.a.23724
Miyara, M., Yoshioka, Y., Kitoh, A., Shima, T., Wing, K., Niwa, A., Parizot, C., Taflin, C., Heike, T., Valeyre, D., Mathian, A., Nakahata, T., Yamaguchi, T., Nomura, T., Ono, M., Amoura, Z., Gorochov, G., Sakaguchi, S., 2009. Functional delineation and differentiation dynamics of human CD4+ T cells expressing the FoxP3 transcription factor. Immunity 19; 30(6):899-911. https://doi.org/10.1016/j.immuni.2009.03.019
Odendahl, M., Mei, H., Hoyer. B F., Jacobi, A M., Hansen, A., Muehlinghaus, G., Berek, C., Hiepe, F., Manz, R., Radbruch, A., Dorner T., 2005. Generation of migratory antigen-specific plasma blasts and mobilization of resident plasma cells in a secondary immune response. Blood 105, 1614-1621. https://doi.org/10.1182/blood-2004-07-2507
Perez-Andres, M., Paiva, B., Nieto. W G., Caraux, A., Schmitz, A., Almeida, J., Vogt., R F Jr., Marti. G E., Rawstron, A C., Van Zelm, M C., Van Dongen, J J., Johnsen, H E., Klein, B., Orfao, A., 2010. Human peripheral blood B-Cell compartments: a crossroad in B-cell traffic. Cytometry B Clin Cytom 78, 47-60. https://doi.org/10.1002/cyto.b.20547
Qian, Y., Wei, C., Eun-Hyung Lee, F., Campbell, J., Halliley, J., Lee, J A., Cai, J., Kong. Y M., Sadat, E., Thomson, E., Dunn, P., Seegmiller, A C., Karandikar. N J., Tipton, C M., Mosmann, T., Sanz, I., Scheuermann, R H., 2010. Elucidation of seventeen human peripheral blood B-cell subsets and quantification of the tetanus response using a density-based method for the automated identification of cell populations in multidimensional flow cytometry data. Cytometry B Clin Cytom. 78 Suppl 1. S69-82. https://doi.org/10.1002/cyto.b.20554.
Roederer, M., Quaye, L., Mangino, M., Beddall. M H., Mahnke, Y., Chattopadhyay, P., Tosi, I., Napolitano, L., Terranova Barberio, M., Menni, C., Villanova, F., Di Meglio, P., Spector, T D., Nestle, F O., 2015. The genetic architecture of the human immune system: a bioresource for autoimmunity and disease pathogenesis. Cell 161, 387-403. https://doi.org/10.1016/j.cell.2015.02.046.
Sandberg Y, Almeida J. Gonzalez M, Lima M. Barcena P, Szczepanski T, Van Gastel-Mol E J, Wind H, Balanzategui A, Van Dongen J J M, Miguel J F, Orfao A. Langerak A W, 2006. TCRγ8+ large granular lymphocyte leukemias reflect the spectrum of normal antigen-selected TCRγ6+ T-cells. Leukemia 20, 505-513.
See, P. C, A. Dutertre, J. Chen, P. Gunther, N. McGovern, S. E. Irac, M. Gunawan, M. Beyer, K. Handler, K. Duan. H1, R. B. Sumatob, N. Ruffin, M. Jouve, E. Gea-Mallorqui, R. C. M. Hennekam, T. Lim, C. C. Yip, M. Wen, B. Malleret, I. Low, N. B. Shadan, C. F. S. Fen, A. Tay, J. Lum, F. Zolezzi, A. Larbi, M. Poidinger, J. K. Y. Chan, Q. Chen, L. Renia, M. Ilaniffa, P. Benaroch, A. Schlitzer, J. L. Schultze, E. W. Newell and F. Ginhoux (2017). “Mapping the human DC lineage through the integration of high-dimensional techniques.” Science 356(6342).
Streitz, M., Miloud, T., Kapinsky, M., Reed, M. R., Magari, R., Geissler, E. K., Hutchinson, J. A., Vogt, K., Schlickeiser, S., Kverneland, A., Meisel, C., Volk, H.-D., Sawitzki, B., 2013. Standardization of whole blood immune phenotype monitoring for clinical trials: panels and methods from the ONE study. Transplant. Res. 2, 17. https://doi.org/10.1186/2047-1440-2-17
Tangye, S. G., Ma, C. S., Brink, R., Deenick, E. K. The good, the bad and the ugly-TFH cells in human health and disease. Nat. Rev. Immunol. 13(6):412-26. https://doi.org/10.1038/nri3447
Van Dongen J J M, Lhermitte L, Böttcher S. Almeida J, Van der Velden V H J. Flores-Montero J, Rawstron A, Asnafi V. Lérevisse Q. Lucio P, Mejstrikova E. Szczepañski T, Kalina T, De Tute R. Bruggemann M. Sedek L. Cullen M, Langerak A W. Mendonça A, Macintyre E, Martin-Ayuso M, Hrusak O. Vidriales M B, Orfao A: EuroFlow Consortium (EU-FP6. LSHB-CT-2006-018708). 2012. EuroFlow antibody panels for standardized n-dimensional flow cytometric immunophenotyping of normal, reactive and malignant leukocytes. Leukemia 26.1908-1975.
Villani, A. C., R. Satija, G. Reynolds, S. Sarkizova, K. Shekhar, J. Fletcher, M. Griesbeck, A. Butler, S. Zheng, S. Lazo, L. Jardine, D. Dixon, E. Stephenson, E. Nilsson, I. Grundberg, D. McDonald, A. Filby, W. Li, P. L. De Jager, O. Rozenblatt-Rosen, A. A. Lane, M. Haniffa, A. Regev and N. Hacohen (2017). “Single-cell RNA-seq reveals new types of human bkxxl dendritic cells, monocytes, and progenitors.” Science 356(6335).
Wingender, G., Kronenberg, M., 2015. OMIP-030: Characterization of human T cell subsets via surface markers. Cytom. Part A 87, 1067-1069. https://doi.org/10.1002/cyto.a.22788
Wong, M. T., Chen, J., Narayanan, S., Lin, W., Anicete, R., Kiaang, H. T. K., De Lafaille, M. A. C., Poidinger, M., Newell, E. W., 2015. Mapping the Diversity of Follicular Helper T Cells in Human Blood and Tonsils Using High-Dimensional Mass Cytometry Analysis. Cell Rep. 11, 1822-1833. https://doi.org/10.1016/j.celrep.2015.05.022
Yin, X. H. Yu, X. Jin, J. Li, H. Guo, Q. Shi, Z. Yin, Y. Xu, X. Wang, R. Liu, S. Wang and L. Zhang (2017). “Human Blood CD1c+ Dendritic Cells Encompass CD5high and CD5low Subsets That Differ Significantly in Phenotype. Gene Expression. and Functions.” J Immunol 198(4): 1553-1564

1.-53. (canceled)

54. A reagent composition for the cytometric immunophenotyping of leukocytes, the reagent composition comprising:
antibodies conjugated to a detectable label, the conjugated antibodies being directed against the following combination of markers: CD141, HLA-DR, CD16, CD33, CD300e, CD303 and CD14,
wherein the antibodies directed against CD300e and CD303 may be conjugated to the same label.

55. The reagent composition of claim 54, further comprising:
conjugated antibodies against one or more of CD5, CD34, and CD45.

56. The reagent composition of claim 54, further comprising:
conjugated antibodies against marker set CD36 and SLAN, and/or marker set CD62L and FcERI.

57. The reagent composition of claim 54, further comprising:
conjugated antibodies against the marker set CD36 and CD192 (CCR2), and/or the marker set CD62L and CD45

58. The reagent composition of claim 54, comprising:
conjugated antibodies against CD141, HLA-DR, CD16, CD33, CD300e, CD303, CD14, CD45, CD5, CD34, CD36, SLAN, CD62L, FcERI and CD192 (CCR2),
wherein the antibodies within the marker sets SLAN/FcERI and CD300e/CD303 can be conjugated to the same label and wherein between the different marker sets the labels are distinguishable.

59. The reagent composition of claim 54, further comprising:
conjugated antibodies against the markers CD1c and/or CD100 and/or Axl.

60. The reagent composition of claim 54, further comprising conjugated antibodies against CD34, CD45, CD64 and CD117, wherein the antibodies for the marker sets CD34 and CD14 can be conjugated to the same detectable label.

61. The reagent composition of claim 60, further comprising:
a conjugated antibody against CD36.

62. The reagent composition of claim 54, further comprising:
conjugated antibodies against CD11b, CD13 and CD35.

63. The reagent composition of claim 54, further comprising:
a conjugated antibody against CD163.

64. The reagent composition of claim 54, further comprising:
conjugated antibodies against CD163 and FcεRI,
wherein the antibodies for the marker set CD34 and CD14 can be conjugated to the same detectable label.

65. The reagent composition of claim 60, supplemented with conjugated antibodies against at least two of the group of markers consisting of CD36, CD11b, CD13, CD35, CD163, FcεRI,
wherein if present the antibodies for the marker set CD34 and CD14 can be conjugated to the same detectable label.

66. A method of using the reagent composition of claim 54 to diagnose, classify, and/or monitor an acute leukemia in a subject, wherein the leukemia is of monocytic or DC origin, the method comprising contacting the reagent composition with a sample taken from the subject.

67. A reagent composition for the cytometric immunophenotyping of leukocytes, the reagent composition comprising:
antibodies conjugated to a detectable label,
wherein the conjugated antibodies are directed against the following combination of markers: CD27, CD45RA or CD45RO, CD62L, CD127, CD3, CD25, CCR10, CD183 (CXCR3), CD196 (CCR6), CD194 (CCR4), CD185 (CXCR5), and CD4.

68. The reagent composition of claim 67, further comprising:
conjugated antibodies against CD8 and/or CD45.

69. The reagent composition of claim 67, further comprising:
conjugated antibodies against CD31 and/or CD95.

70. The reagent composition of claim 67, further comprising:
conjugated antibodies against (i) one or more of CD278 (ICOS), CD279 (PD1) and HLA-DR and/or (ii) conjugated antibodies against mutually exclusive TcR-C131 and/or TcR-C132 epitopes.

71. The reagent composition of claim 67, further comprising:
conjugated antibodies against
(i) TCRγδ
(ii) CD16, CD56 and/or CD335 to identify NK cells; and
(iii) cytotoxic-related markers.

72. The reagent composition of claim 67, further comprising:
conjugated antibodies against the markers CD2, CD279, CD7, CD26 and CD28, optionally further HLA-DR, and/or cyTCL1.

73. The reagent composition of claim 67, further comprising:
conjugated antibodies against (i) TcR-Vβ and/or TcR-Vα domains and (ii) TcRγ6.

74. The reagent composition of claim 67, further comprising:
conjugated antibodies against (i) TcR-Vδ and/or TcR-Vγ domains and (ii) TcRαβ.

75. The reagent composition of claim 67, further comprising:
one or more antigen-specific peptides in MEW molecules for detection and enumeration of antigen-specific T-cells, which MEW molecules are included in multimeric constructs that may be directly or indirectly conjugated to a detectable label.

76. A set of reagent compositions comprising:
(i) the reagent composition of claim 67; and
(i) a reagent composition comprising conjugated antibodies against CD3, CD4, CD8, CD27, CD45, CD62L, CD45RA or CD45RO, CD16, CD28, CD56, CD57, TCRγ6, optionally supplement with conjugated antibodies against the markers CD335 and/or granzyme B.

77. A set of reagent compositions, comprising:
(i) the reagent composition of claim 75; and
(ii) a reagent composition comprising conjugated antibodies directed against the markers CD27, CD45RA, CD45RO, CD8, CD62L, CD127, CD3, CD25, CD7, HLA-DR, CD45, CD4, CD11c, CD16, CD30, CD56, CD57, CD94, CD5, TcRγδ, optionally supplemented with conjugated antibodies directed against granzyme B and/or perforin.

78. A reagent composition for the cytometric immunophenotyping of leukocytes, the reagent composition comprising:
antibodies conjugated to a detectable label,
wherein the conjugated antibodies are directed against the following combination of markers: CD20, CD38, CD62L, and CD138, optionally combined with CD19.

79. The reagent composition of claim 78, further comprising conjugated antibodies against Igkappa and Iglambda, wherein the antibodies against Igkappa and Iglambda may be conjugated to the same or to a distinct label.

80. The reagent composition of claim 78, further comprising a conjugated antibody against cyIgM.

81. The reagent composition of claim 78, further comprising:
conjugated antibodies against IgA and IgG,
wherein the antibodies directed against IgA and IgG may be conjugated to the same label.

82. The reagent composition of claim 78, further comprising:
a conjugated antibody against IgD, and
conjugated antibodies against IgM and CD45.

83. The reagent composition of claim 82, further comprising:
conjugated antibodies against CD27 and CD5,
wherein the antibodies against CD5 and CD138 may be conjugated to the same label.

84. The reagent composition of claim 78, further comprising:
conjugated antibodies against IgD, IgG1, IgG2, IgG3, IgG4, IgA1 and IgA2 subclasses,
wherein the antibodies directed against these IgD, IgG and IgA subclasses may be conjugated in part to the same label.

85. The reagent composition of claim 81, further comprising a conjugated antibody against IgE.

86. The reagent composition of claim 84, further comprising:
conjugated antibodies against CD27 and CD5,
wherein the antibodies against CD5 and CD138 may be conjugated to the same label.

87. The reagent composition of claim 78, further comprising:
one or more antigens or allergens for detection and enumeration of antigen-specific or allergen-specific plasma cells and B-cells, which antigen(s) or allergen(s) may be directly or indirectly conjugated to a detectable label.

88. The reagent composition of claim 54, wherein the antibodies are conjugated to a fluorochrome.

89. The reagent composition of claim 54, wherein the antibodies are conjugated to a metal-isotope.

90. A reagent composition comprising an admixture of the conjugated antibodies as defined in two or more of
(i) a reagent comprising: antibodies conjugated to a detectable label, the conjugated antibodies being directed against the following combination of markers: CD141, HLA-DR, CD16, CD33, CD300e, CD303 and CD14, wherein the antibodies directed against CD300e and CD303 may be conjugated to the same label;
(ii) a reagent composition comprising: antibodies conjugated to a detectable label, wherein the conjugated antibodies are directed against the following combination of markers: CD27, CD45RA or CD45RO, CD62L, CD127, CD3, CD25, CCR10, CD183 (CXCR3), CD196 (CCR6), CD194 (CCR4), CD185 (CXCR5), and CD4; and
(iii) the reagent composition of claim 78.

91. A set of reagent compositions, comprising:
a combination of two or more of
(i) a reagent comprising: antibodies conjugated to a detectable label, the conjugated antibodies being directed against the following combination of markers: CD141, HLA-DR, CD16, CD33, CD300e, CD303 and CD14, wherein the antibodies directed against CD300e and CD303 may be conjugated to the same label;
(ii) a reagent composition comprising: antibodies conjugated to a detectable label, wherein the conjugated antibodies are directed against the following combination of markers: CD27, CD45RA or CD45RO, CD62L, CD127, CD3, CD25, CCR10, CD183 (CXCR3), CD196 (CCR6), CD194 (CCR4), CD185 (CXCR5), and CD4; and
(iii) the reagent composition of claim 78.

92. A diagnostic kit for cytometric immunophenotyping of leukocytes, the diagnostic kit comprising:
the reagent composition of claim 54, together with buffer, and/or control samples.

93. A diagnostic kit for multi-color flow cytometric immunophenotyping of leukocytes, the diagnostic kit comprising:
the reagent composition of claim 88, together with buffer, and/or control samples.

94. A diagnostic kit for mass cytometric immunophenotyping of leukocytes, the diagnostic kit comprising:
the reagent composition of claim 89, together with buffer, and/or control samples.

95. A method of using the diagnostic kit of claim 92 in multiparameter cytometry-based monitoring of the immune status and/or the effect of an immune modulatory treatment.

96. The reagent composition of claim 58, being a 12-color reagent composition comprising conjugated antibodies against CD141, HLA-DR, CD16, CD33, CD300e, CD303, CD14, CD45, CD5, CD34, CD36, SLAN, CD62L, FcERI and CD192(CCR2), wherein the antibodies within the marker sets SLAN/FcERI, CD300e/CD303, and CD14/CD34 are conjugated to the same label and wherein between the different marker sets the labels are distinguishable.

97. The reagent composition of claim 68, being a 12-color reagent composition comprising conjugated antibodies against CD27, CD62L, CD127, CD3, CD25, CCR10, CD183 (CXCR3), CD196 (CCR6), CD194 (CCR4), CD185 (CXCR5), CD4, CD45, and CD45RA or CD45RO,
wherein the antibodies against CD4 and CD45 are conjugated to the same label.

98. The reagent composition of claim 97 together with a 12-color reagent composition comprising conjugated antibodies directed against the markers CD27, CD62L, CD3, CD45, CD4, CD45R0 or CD45RA, CD8, CD16, CD28, CD56, CD57, TcRγ6 and granzyme-B,
wherein the antibodies against CD4 and CD45 are conjugated to the same label.

99. The reagent composition of claim 82, being a 12-color reagent composition comprising conjugated antibodies directed against the markers CD19, CD20, CD38, CD62L, CD138, CD21, CD27, CD45, IgM, IgD, IgG1, IgG2, IgG3, IgG4, IgA1 and IgA2,
wherein three IgD, IgG and IgA subclass antibodies are present in the form of two different conjugates, each conjugate carrying a distinct detectable label, while one of them is present in the form of three different conjugates, each conjugate carrying a distinct detectable label.

100. A method of monitoring the effect of an immune modulatory treatment selected from the group consisting of classical immune suppressive treatments, cellular treatments, gene therapy, stem cell transplantation, CAR T-cell treatment, check point inhibitors, antibody treatments, and vaccinations, the method comprising:
utilizing the diagnostic kit of claim 92 to analyze a sample taken from a subject undergoing immune modulatory treatment.

101. A cytometric method for monitoring the immune status and/or the effect of an immune modulatory treatment of a subject, the method comprising the steps of:
(a) contacting an aliquot of a biological sample comprising leukocytes obtained from the subject with a reagent composition of claim 54;
(b) analyzing leukocytes in the aliquot in a flow or mass cytometer; and
(c) storing and evaluating the data obtained.

102. The method according to claim 101, wherein the sample is peripheral blood, bone marrow, cord blood, tissue sample such as lymph nodes, adenoid, spleen, or liver, or other type of body fluid such as cerebrospinal fluid, vitreous fluid, synovial fluid, fine needle aspirate, pleural effusions, or ascitic fluid.

103. The method according to claim 101, wherein step (c) comprises combining the immunophenotypic information of selected cell populations from multiple tubes according to the so-called nearest neighbor calculations in which individual cells from one aliquot of a sample are matched with corresponding individual cells from another aliquot of the same sample, according to their markers and scatter profile.

104. The method according to claim 101, further comprising:
using software for data integration and multidimensional analysis of flow cytometry files.
